(19) United States
(12) Patent Application Publication
(10) Pub. No.: US 2022/0397579 A1
(43) Pub. Date: Dec. 15, 2022

(54) METHODS AND KITS FOR QUANTITATING RADIATION EXPOSURE

(71) Applicant: MESO SCALE TECHNOLOGIES, LLC., Rockville, MD (US)

(72) Inventors: Sunsanee Kanjananimmanont, Germantown, MD (US); Sudeep Kumar, Basking Ridge, NJ (US); Navaratnam Manjula, Rockville, MD (US); Simone Barbero, Gaithersburg, MD (US); Martin Stengelin, Gaithersburg, MD (US); George Sigal, Rockville, MD (US)

(21) Appl. No.: 17/773,896

(22) PCT Filed: Nov. 4, 2020
(86) PCT No.: PCT/US2020/058866

§ 371 Date: May 3, 2022

Related U.S. Application Data
(60) Provisional application No. 62/947,731, filed on Dec. 13, 2019.
(60) Provisional application No. 62/931,053, filed on Nov. 5, 2019.

(51) Int. Cl. G01N 33/68; G01N 33/543

(52) U.S. Cl. CPC G01N 33/6893; G01N 33/54306; G01N 2800/40; G01N 2800/60

(57) ABSTRACT

The invention relates to methods and kits for quantitating radiation exposure in a subject exposed to radiation, at risk of exposure to radiation or suspected of having been exposed to radiation. In embodiments, the present disclosure provides multiplexed immunoassays for quantifying amounts of biomarkers for assessing radiation exposure in a sample. Also provided herein are kits for performing the multiplexed assays.

### STATEMENT REGARDING FEDERALLY-SPONSORED RESEARCH

This invention was made with federal support under HHSN272201700013C awarded by the Department of Health and Human Services. The U.S. government has certain rights in the invention.

### FIELD OF THE INVENTION

The invention relates to methods and kits for quantitating radiation exposure in a subject exposed to radiation, at risk of exposure to radiation or suspected of having been exposed to radiation.

### BACKGROUND

Explosion of an improvised nuclear device (IND) or other radiation producing event in a major U.S. city could lead to the exposure of tens of thousands of individuals to radiation levels sufficient to cause acute illness. Currently, no diagnostic tools exist that could be used in such an emergency to test the large number of potentially exposed individuals, assess their radiation exposure, and aid in selecting the appropriate course of treatment.

### SUMMARY OF THE INVENTION

In embodiments, the disclosure provides a multiplexed immunoassay method comprising: quantifying the amounts of at least four biomarkers in a biological sample, wherein the at least four biomarkers comprise (a) IL-15, (b) CD5, (c) Flt-3L, and (d) salivary amylase, wherein the quantifying comprises measuring the concentrations of the at least four biomarkers in a multiplexed assay format to simultaneously measure the concentrations of the least four biomarkers in a biological sample wherein the multiplexed immunoassay comprises: a) combining, in one or more steps: (i) the biological sample; (ii) at least a first, second, third, and fourth binding reagent, wherein the first, second, third, and fourth binding reagent is a binding partner of IL-15, CD5, Flt-3L, and salivary amylase, respectively; b) forming at least a first, second, third, and fourth binding complex comprising the binding reagents and the biomarkers; c) measuring the concentration of the biomarkers in each of the complexes.

In embodiments, the disclosure provides a multiplexed immunoassay method comprising: quantifying the amounts of at least four biomarkers in a biological sample, wherein the at least four biomarkers comprise IL-15, CD5, Flt-3L, and salivary amylase, wherein the quantifying comprises measuring the concentrations of the at least four biomarkers in a multiplexed assay format to simultaneously measure the concentrations of at least four biomarkers in the biological sample, wherein the multiplexed immunoassay comprises: a) combining, in one or more steps: (i) the biological sample; (ii) at least a first antibody to IL-15; a first antibody to CD5; a first antibody to Flt-3L; and a first antibody to salivary amylase, wherein each of the first antibodies is immobilized on separate binding domains; (iii) at least a second antibody to IL-15; a second antibody to CD5; a second antibody to Flt-3L; and a second antibody to salivary amylase, wherein each second antibody is connected to a detectable label; b) forming at least a first, second, third, and fourth binding complex on an at least first, second, third, and fourth binding domains comprising at least IL-15, CD5, Flt-3L, and salivary amylase, and the first and second antibodies for their respective biomarker; c) measuring the concentration of the at least IL-15, CD5, Flt-3L, and salivary amylase on the at least first, second, third, and fourth binding domains.

In embodiments, the disclosure further provides a method of determining radiation exposure in a human, comprising a) conducting the multiplexed immunoassay as described herein on a biological sample of a human, b) detecting the concentration of biomarker IL-15, biomarker CD5, biomarker Flt-3L, and biomarker salivary amylase, c) determining if: (i) the concentration of biomarker IL-15 is higher compared to a control; (ii) the concentration of biomarker CD5 is lower compared to a control; (iii) if the concentration of biomarker Flt-3L is higher compared to a control; (iv) if the concentration of salivary amylase is higher or the same compared to a control, wherein if any of (i), (ii), (iii) or (iv) is true, reporting that the human has been exposed to radiation, wherein the control of (i), (ii), (iii), and (iv) is from a human who has not been exposed to radiation. The roles of IL-15, CD5, Flt-3L, and salivary amylase in radiation response are described herein. In embodiments, the determining is performed by an immunoassay, e.g., a multiplexed immunoassay described herein.

In further embodiments, the disclosure provides a method of determining radiation exposure in a human, comprising a) detecting CD5 in a biological sample of a human, b) determining if a concentration of CD5 in the biological sample is lower than a control concentration of CD5 in a non-irradiated control sample, c) if the concentration in the biological sample is lower than in the non-irradiated control sample, reporting that the human was exposed to radiation.

In yet further embodiments, the present disclosure further provides a kit comprising, in one or more vials, containers, or components: (a) a surface comprising at least a first, second, third, and fourth binding reagent immobilized on an associated first, second, third, and fourth binding domain, wherein the first, second, third, and fourth binding reagent is a binding partner of IL-15, CD5, Flt-3L, and salivary amylase, respectively; (b) a detection reagent that specifically binds to biomarker IL-15; (c) a detection reagent that specifically binds to CD5; (d) a detection reagent that specifically binds to Flt-3L; and (e) a detection reagent that specifically binds to salivary amylase.

In embodiments, IL-18 is added as a biomarker, or IL-15 is replaced with IL-18. In embodiments, the biomarkers are human biomarkers.

### BRIEF DESCRIPTION OF THE DRAWINGS

The following drawings form part of the present specification and are included to further demonstrate exemplary embodiments of certain aspects of the present invention.

FIG. 1 relates to embodiments of Example 1. FIG. 1 shows an example immunoassay for salivary amylase (AMY1A), a high abundance biomarker, in standard (light grey) and desensitized (dark grey) formats. The crosses on the plot indicate normal AMY1A level in human plasma, while the vertical line represents the AMY1A level in plasma with the highest dose of radiation.

FIGS. 2A-2K relate to embodiments of Example 2. FIGS. 2A-2K show, respectively, calibration curves of immunoassays for CD5, CD27, CD177, CD20, Flt-3L, IL-12/23, IL-15, IL-18, thyroid peroxidase (TPO), erythropoietin (EPO), and AMY1A. Bold-lined and thin-lined crosses indicate measured levels for a set of normal plasma samples from 18 human donors and 18 nonhuman primate (NHP) models, respectively, in FIGS. 2A-2K.

FIGS. 3A-3B relate to embodiments of Example 2. FIG. 3A shows various example assay parameters of multiplexed biomarker panels, including the coefficient of variation (column labeled “Precision”), assay measurement range (limit of detection (LOD), lower limit of quantitation (LLOQ), and upper limit of quantitation (ULOQ)), and range of biomarker concentration values measured for human and NHP plasma samples. FIG. 3B shows results of measured biomarker concentrations in human and NHP plasma samples relative to the LOD (bold-lined bars), LLOQ and ULOQ (thin-lined bars). Arrows above each column represent the direction in which the concentration is expected to change after radiation exposure.

FIGS. 4A-4B relate to embodiments of Example 3. FIG. 4A shows the linearity-on-dilution assessment of normal plasma samples diluted in assay calibrator diluent. Analytes marked with asterisk (*) had normal levels near the LLOQ. FIG. 4B shows the spike recovery assessment of a purified calibrator biomarker spiked into plasma samples.

FIG. 5 relates to embodiments of Example 4. FIG. 5 shows a summary of the samples used in a NHP radiation study. Six animals were exposed to each dose condition shown in the row under “Dose (Gy”), and plasma samples were collected at different time points before (0 days) and after radiation as shown in the first column. The numbers in each cell indicate the number of samples tested for each dose-time combination.

FIG. 6 relates to embodiments of Example 4.1. FIG. 6 shows changes in CD5, CD20, CD27, and CD177 biomarkers in NHP plasma as a function of time (for the first 9 days) and radiation dose. Error bars represent the standard deviation in the measured biomarker level across the different replicate animals. The lowest horizontal line in each plot represents the assay LOD. The upper and middle horizontal lines in each plot represents the quantitation range defined by the LLOQ (middle line) and ULOQ (upper line).

FIG. 7 relates to embodiments of Example 4.2. FIG. 7 shows changes in IL-12, IL-15, IL-18, Flt-3L, EPO, and TPO biomarkers in NHP plasma as a function of time (for the first 9 days) and radiation dose. Error bars represent the standard deviation in the measured biomarker level across the different replicate animals. The lowest horizontal line in each plot represents the assay LOD. The upper and middle horizontal lines in each plot represents the quantitation range defined by the LLOQ (middle line) and ULOQ (upper line).

FIG. 8 relates to embodiments of Example 4.3. FIG. 8 shows changes in AMY1A, AMY2A measured using a desensitized assay in undiluted (neat) samples, and AMY2A also measured in diluted samples in NHP plasma as a function of time (for the first 9 days) and radiation dose. Error bars represent the standard deviation in the measured biomarker level across the different replicate animals. The lowest horizontal line in each plot represents the assay LOD. The upper and middle horizontal lines in each plot represents the quantitation range defined by the LLOQ (middle line) and ULOQ (upper line).

FIG. 9 relates to embodiments of Example 5. FIG. 9 shows a summary of the subjects in the stem cell transplant (SCT) patient study. AML: acute myeloid leukemia; ALL: acute lymphoblastic leukemia; KGF: keratinocyte growth factor.

FIG. 10 relates to embodiments of Example 5.1. FIG. 10 shows changes in CD5, CD20, CD27, and CD177 biomarkers in human plasma from SCT patients as a function of time during fractionated total body irradiation (TBI) regimen. Each curve represents samples from a different patient. The two horizontal dashed lines near the top and bottom of each plot provide the quantitation range defined by the LLOQ (lower line) and ULOQ (upper line). The two horizontal lines in the middle of each plot represent the ±1 standard deviation range for a set of 10 normal human plasma samples tested at the same time as the SCT patient samples.

FIG. 11 relates to embodiments of Example 5.2. FIG. 11 shows changes in IL-12, IL-15, IL-18, Flt-3L, EPO, and TPO biomarkers in human plasma from SCT patients as a function of time during fractionated total body irradiation (TBI) regimen. Each curve represents samples from a different patient. The two horizontal dashed lines near the top and bottom of each plot provide the quantitation range defined by the LLOQ (lower line) and ULOQ (upper line). The two horizontal lines in the middle of each plot represent the ±1 standard deviation range for a set of 10 normal human plasma samples tested at the same time as the SCT patient samples.

FIG. 12 relates to embodiments of Example 5.3. FIG. 12 shows changes in salivary amylase (AMY1A), C-reactive protein (CRP), and cardiac troponin (cTnl) in human plasma from SCT patients as a function of time during fractionated total body irradiation (TBI) regimen. Each curve represents samples from a different patient. The two horizontal dashed lines near the top and bottom of each plot provide the quantitation range defined by the LLOQ (lower line) and ULOQ (upper line). The two horizontal lines in the middle of each plot represent the ±1 standard deviation range for a set of 10 normal human plasma samples tested at the same time as the SCT patient samples.

FIGS. 13A-13C relate to embodiments of Example 6. FIG. 13A shows a training data set of a biomarker concentration plotted against dose (Gy) and time (days post irradiation). FIG. 13B shows an exemplary prediction model based on measured concentrations of two biomarkers, wherein the best predicted dose falls between the best individual matches for each of the two biomarkers. FIG. 13C shows the root mean square error (RMSE) in dose prediction across all test samples for all possible combinations of biomarkers.

FIGS. 14A-14B relate to embodiments of Example 7. FIGS. 14A and 14B show NHP and human plasma samples, respectively, tested with a five-biomarker panel in a multiplexed assay. In FIG. 14A, error bars represent the standard deviation in the measured biomarker level across the different replicate animals. The lowest horizontal line in each plot represents the assay LOD. The upper and middle horizontal lines in each plot represents the quantitation range defined by the LLOQ (middle line) and ULOQ (upper line). In FIG. 14B, each curve represents samples from a different patient. The two horizontal dashed lines near the top and bottom of each plot provide the quantitation range defined by the LLOQ (lower line) and ULOQ (upper line). The two horizontal lines in the middle of each plot represent the ±1 standard deviation range for a set of 10 normal human plasma samples tested at the same time as the SCT patient samples.

FIGS. 15A-15B relate to embodiments of Example 7. FIGS. 15A and 15B show plots of predicted vs. actual radiation doses for NHP samples tested with five- and six-biomarker panels, respectively.

FIG. 16 relates to embodiments of Example 8. FIG. 16 shows the average coefficient of variation (CV) for control samples measured in 15 assays plates tested in 5 processing batches on 3 different days using an automated ultra-high throughput (UHT) system.

FIG. 17 relates to embodiments of Example 9. FIG. 17 shows a comparison of the assay parameters (LOD, LLOQ, and ULOQ) for manual and UHT assay formats.

FIGS. 18A-18C relate to embodiments of Example 10. FIG. 18A shows results of a multiplexed assay performed using a five-biomarker panel on NHP plasma samples obtained from individuals subjected to radiation. FIG. 18B shows a plot of biomarker levels in human plasma samples from subjects in different categories, e.g., age or disease. FIG. 18C shows results of a multiplexed assay performed using a five-biomarker panel on human plasma samples obtained from individuals subjected to radiation.

FIG. 19 illustrate an example NHP dose response data set that can be used to train a cost function algorithm and a linear model algorithm, as disclosed in embodiments herein.

FIGS. 20A and 20B illustrate an example of the sensitivity and specificity plot for the cost function algorithm and the linear model algorithm. FIG. 20A illustrates an example of random sub-sampling to measure the specificity and sensitivity as a function of a cutoff value using the cost function algorithm and FIG. 20B illustrates an example of random sub-sampling to measure the specificity and sensitivity as a function of a cutoff value using the linear model algorithm.

FIGS. 21A and 21B illustrate an example of the accuracy of the cost function algorithm and the linear model algorithm, as described herein. FIG. 21A illustrates an example of the accuracy of the cost function algorithm and FIG. 21B illustrates an example of the accuracy of the linear model algorithm.

FIG. 22 illustrates the data from human patients used to test the cost function algorithm and the linear model algorithm.

FIG. 23 illustrates an example of the results for the test of the cost function algorithm and the linear model algorithm using the data from FIG. 22.

FIG. 24 contain tables showing the observed specificities for the different classes of subjects as shown in FIG. 23. The Table A shows predicted specificity for the cost function algorithm, and the Table B shows predicted specificity for the linear model algorithm.

FIG. 25 illustrates data from a human stem cell transplant (SCT).

FIG. 26A illustrates an example of dose prediction for SCT patient samples as a function of total dose for the cost function algorithm and FIG. 26B illustrates an example of dose prediction for SCT patient samples as a function of total dose for the linear model algorithm.

FIG. 27 shows an alternative dosing regimen for the NHP study described in embodiments of Example 4.4.

FIG. 28 relates to embodiments of Example 4.4. FIG. 28 contains a table showing the specificity and sensitivity of classification using the regression model.

FIG. 29 relates to embodiments of Example 11. FIG. 29 contains a table of components that are commonly present in sample matrices that can interfere with biomarker level measurements, also known as interferents. The components in FIG. 29 are organized by category. The expected highest level of each interferent in a plasma sample is shown as the Target Concentration (1×). Each interferent was spiked into plasma samples at four times the target concentration, shown as the 4× Screening concentration.

FIG. 30 relates to embodiments of Example 11. FIG. 30 shows the results of five-biomarker assay panels with four plasma samples that were spiked with the interferents in FIG. 29 at 4× Screening concentration. The five-biomarker assay panel measured CD20, IL-15, AMY1A, CD5, and Flt-3L.

FIG. 31 relates to embodiments of Example 11. FIG. 31 shows the results of titrating the concentrations of interferents hemolysate, lipid, unconjugated bilirubin, and conjugated bilirubin into plasma samples at decreasing spike concentrations: 4×, 2×, 1×, 0.5×, 0.25×, 0.125×, and 0×, where 1× represents the expected highest level of the interferent in a plasma sample.

FIG. 32 relates to embodiments of Example 13. FIG. 32 shows the results of a stability test for an assay plate containing a five-biomarker assay panel for measuring levels of CD20, IL-15, AMY1A, CD5, and Flt-3L. The plates were stored in open air at either 25° C. (22 to 27° C.) or 37° C. (35 to 40° C.), then used to measure biomarker concentrations in two control samples and three plasma samples. The measured concentrations were plotted after normalization to a plate that was stored at 4° C. and used immediately following removal from storage (4° C., 0 hr condition).

FIG. 33 relates to embodiments of Example 13. FIG. 33 shows the results of a stability test for control samples containing known amounts of CD20, IL-15, AMY1A, CD5, and Flt-3L. Lyophilized control samples were reconstituted and stored for up to 24 hours at 4° C. or 25° C., then analyzed using the five-biomarker assay panel for measuring levels of CD20, IL-15, AMY1A, CD5, and Flt-3L. The measured concentrations were compared to the concentrations measured immediately after reconstitution (0 hr condition).

FIG. 34 relates to embodiments of Example 13. FIG. 34 shows the results of a stability test for calibration standards for each of the biomarkers CD20, IL-15, AMY1A, CD5, and Flt-3L. Lyophilized calibration standards were reconstituted and stored for up to 24 hours at 4° C. or 25° C. The calibration standards were then used in the five-biomarker assay panel for measuring levels of CD20, IL-15, AMY1A, CD5, and Flt-3L to determine concentrations of the biomarkers in two control samples and three plasma samples. The measured concentrations of biomarkers in the control samples and plasma samples were compared to the concentrations that were measured using calibration standards that were used immediately after reconstitution (0 hr condition).

FIG. 35 relates to embodiments of Example 14. FIG. 35 shows the temperature and length of time that plasma samples were stored, prior to testing for stability by measuring biomarker levels using a five-biomarker assay panel for CD20, IL-15, AMY1A, CD5, and Flt-3L.

FIGS. 36A-36C relate to embodiments of Example 14. FIGS. 36A-36C show the results of a stability test for plasma samples. Ten different plasma samples were stored according to the conditions shown in FIG. 35. FIG. 36A shows the measured concentrations of CD20, IL-15, AMY1A, CD5, and Flt-3L in plasma samples 1-4. FIG. 36B shows the measured concentrations of CD20, IL-15, AMY1A, CD5, and Flt-3L in plasma samples 5-8. FIG. 36C shows the measured concentrations of CD20, IL-15, AMY1A, CD5, and Flt-3L in plasma samples 9-10.

FIG. 37 relates to embodiments of Example 15.1. FIG. 37 shows the results of a control experiment using a multiplexed five-biomarker panel assay for CD20, IL-15, AMY1A, CD5, and Flt-3L on a positive control sample, a negative control sample, and a pooled plasma sample. Measurements were performed on 21 assay plates over the course of 7 days with the samples in duplicate. The measured concentration is shown after normalization to the median value across the runs, and the inset table shows the measured coefficient of variations (CVs) for each control/assay combination across the experiment. The table also shows the percentage of the controls that provided the correct dose classification result (the negative and pooled plasma control should be classified as having a dose <2 Gy and the positive control should be classified as having a dose ≥2 Gy).

FIG. 38 relates to embodiments of Example 15.2. FIG. 38 shows the results of a multiplexed five-biomarker panel assay for CD20, IL-15, AMY1A, CD5, and Flt-3L and an AMY2A assay performed on NHP plasma samples that were subjected to different doses of radiation.

FIG. 39 relates to embodiments of Example 15.2. FIG. 39 shows the performance accuracy of the dose assessment algorithms (cost function or error minimization and linear regression), with the plots showing predicted dose as a function of actual dose with points colored based on time from exposure. The tables provide the classification accuracy for all negative and positive samples, or stratified by dose (top: error minimization algorithm; bottom: linear regression algorithm).

FIG. 40 relates to embodiments of Example 15.3. FIG. 40 shows the results of a multiplexed five-biomarker panel assay for CD20, IL-15, AMY1A, CD5, and Flt-3L and an AMY2A assay performed on NHP plasma samples that were subjected to 0 or 6 Gy of radiation and subjected to no treatment (control arm) or 10 μg/kg G-CSF daily, starting at day 1 post-exposure (treatment arm).

FIG. 41 relates to embodiments of Example 15.3. FIG. 41 shows the performance accuracy of the dose assessment algorithms (cost function or error minimization and linear regression), with the plots showing predicted dose as a function of actual dose and whether the study animals received G-CSF after irradiation. The tables provide the classification accuracy for all negative and positive samples, stratified by drug treatment arm (top: error minimization algorithm; bottom: linear regression algorithm).

FIG. 42 relates to embodiments of Example 15.4. FIG. 42 shows the results of a multiplexed five-biomarker panel assay for CD20, IL-15, AMY1A, CD5, and Flt-3L performed on human plasma samples from normal or special populations based on age, injury, disease or special condition.

FIG. 43 relates to embodiments of Example 15.4. FIG. 43 shows the specificity of the dose assessment algorithms (cost function or error minimization and linear regression). The tables show the observed specificities for the different classes of subjects (top: error minimization algorithm; bottom: linear regression algorithm).

FIG. 44 relates to embodiments of Example 15.5. FIG. 44 shows the results of a multiplexed five-biomarker panel assay for CD20, IL-15, AMY1A, CD5, and Flt-3L performed on human plasma samples from patients having been subjected to total body irradiation (TBI) prior to stem cell transplant therapy.

FIG. 45 relates to embodiments of Example 15.5. FIG. 45 shows the performance of dose assessment algorithms (cost function or error minimization and linear regression), with the dose prediction for SCT patient samples as a function of total dose. The tables show specificity and sensitivity for the full data set, and after removing data from subjects with undetectable CD20 at baseline ((top: error minimization algorithm; bottom: linear regression algorithm).

FIG. 46 illustrates an exemplary assay surface described in embodiments herein. FIG. 46 shows a well of an exemplary 96-well assay plate, comprising ten distinct binding domains (“spots”).

### DETAILED DESCRIPTION OF THE INVENTION

In embodiments, the present disclosure provides multiplexed immunoassays for quantifying amounts of at least four biomarkers in a sample. In embodiments, the disclosure also provides kits for performing the multiplexed assays.

### I. Definitions

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

### II. Overview

Measurement of biomarker values and levels before and after a particular event, e.g., cellular or environmental event, may be used to gain information regarding an individual's response to the event. For example, samples or model organisms can be subjected to stress- or disease-inducing conditions, or a treatment or prevention regimen, and a particular biomarker can then be detected and quantitated in order to determine its changes in response to the condition or regimen. However, the opposite, i.e., measuring biomarker values and levels to determine whether an organism has been subjected to stress- or disease-inducing condition, tends to be much more complicated, as changes in the levels of a single biomarker typically cannot be definitively associated with a particular condition.

While single biomarkers generally do not provide sufficient information, e.g., for prediction and/or diagnosis of a disease or condition, certain combinations of biomarkers may be used to provide a strong prediction and/or diagnosis. Although a linear combination of biomarkers (i.e., the combination comprises biomarkers that individually provide a relatively strong correlation) can be utilized, linear combinations may not be available in many situations, for example, when there are not enough biomarkers available and/or with strong correlation. In alternative approaches, a biomarker combination is selected such that the combination is capable of achieving improved performance (i.e., prediction or diagnosis) compared with any of the individual biomarkers, each of which may not be a strong correlator on its own. Biomarkers for inclusion in a biomarker combination can be selected for based on their performance in different individuals, e.g., patients, wherein the same biomarker may not have the same performance in different individuals, but when combined with the remaining biomarkers, provide an unexpectedly strong correlation for prediction or diagnosis in a population. For example, Bansal et al., Statist Med 32: 1877-1892 (2013) describe methods of determining biomarkers to include in such a combination, noting in particular that optimal combinations may not be obvious to one of skill in the art, especially when subgroups are present or when individual biomarker correlations are different between cases and controls. Thus, selecting a combination of biomarkers for providing a consistent and accurate prediction and/or diagnosis can be particularly challenging and unpredictable.

Even when a suitable combination of biomarkers is determined, utilizing the combination of biomarkers in an assay poses its own set of difficulties. For example, detecting and/or quantitating each biomarker in the combination in its own separate assay may not be feasible with small samples, and using a separate assay to measure each biomarker in a sample may not provide consistent and comparable results. Furthermore, running an individual assay for each biomarker in a combination can be a cumbersome and complex process that can be inefficient and costly.

A multiplexed assay that can simultaneously measure the concentrations of multiple biomarkers can provide reliable results while reducing processing time and cost. Challenges of developing a multi-biomarker assay (such as, e.g., a multiplexed assay described in embodiments herein) include, for example, determining compatible reagents for all of the biomarkers (e.g., capture and detection reagents described herein should be highly specific and not be cross-reactive; all assays should perform well in the same diluents); determining concentration ranges of the reagents for consistent assay (e.g., comparable capture and detection efficiency for the assays described herein); having similar levels in the condition and sample type of choice such that the levels of all of the biomarkers fall within the dynamic range of the assays at the same dilution; minimizing non-specific binding between the biomarkers and binding reagents thereof or other interferents; and accurately and precisely detecting a multiplexed output measurement.

In embodiments, the present disclosure provides a multi-biomarker assay for determining radiation exposure. Individuals exposed to ionizing radiation, either as a result of a major radiological or nuclear event, during medical treatment, or as a result of an accidental exposure, may suffer from systemic and organ-specific damage. For example, acute effects of high-dose ionizing radiation (>2 Gy) include depletion of specific types of peripheral blood cells, immune suppression, mucosal damage, and potential injury to other sites such as bone and bone marrow niche cells, gastrointestinal system, lungs kidneys, and central nervous system. Exposure to low or moderately high doses (1-3 Gy) of ionizing radiation can result in increased mortality, especially if accompanied by physical injuries, opportunistic infections, and/or hemorrhage. Long-term effects include dysfunction or fibrosis in a wide range of organs and tissues, cataracts, and a higher risk of cancer. In many cases, the effects of radiation exposure can be mitigated by early triage and treatment.

Although radioactive material can be detected using instruments, assessment of radiation dose or injury that an individual has received is more difficult. Moreover, current medical countermeasures for radiation injuries are often expensive, labor-intensive, and time-consuming to administer and monitor, have limited availability, and can be associated with serious toxicities, they should only be administered to individuals most likely to benefit from their use. Thus, fast and accurate radiation dose and tissue injury assessment can greatly facilitate identification of exposed individuals who may benefit from early medical intervention.

Current methods of diagnosing radiation exposure, e.g., the dicentric chromosome assay, can be labor intensive and slow to produce results, and no diagnostic method is available to reliably discriminate levels of radiation exposure based on samples collected at a single time point. It was discovered by the present inventors that radiation exposure can be assessed using a combination of biomarkers. Thus, in embodiments, the present disclosure provides a multiplexed assay method for detecting and/or quantitating biomarkers related to radiation exposure.

In embodiments, the disclosure provides a multiplexed immunoassay method comprising: quantifying the amounts of at least four biomarkers in a biological sample, wherein the at least four biomarkers comprise (a) IL-15, (b) CD5, (c) Flt-3L, and (d) salivary amylase, wherein the quantifying comprises measuring the concentrations of the at least four biomarkers in a multiplexed assay format to simultaneously measure the concentrations of the least four biomarkers in a biological sample wherein the multiplexed immunoassay comprises: a) combining, in one or more steps: (i) the biological sample; (ii) at least a first, second, third, and fourth binding reagent, wherein the first, second, third, and fourth binding reagent is a binding partner of IL-15, CD5, Flt-3L, and salivary amylase, respectively; b) forming at least a first, second, third, and fourth binding complex comprising the binding reagents and the biomarkers; c) measuring the concentration of the biomarkers in each of the complexes.

In embodiments, the disclosure further provides a multiplexed immunoassay method comprising: quantifying the amounts of at least four biomarkers in a biological sample, wherein the at least four biomarkers comprise IL-15, CD5, Flt-3L, and salivary amylase, wherein the quantifying comprises measuring the concentrations of the at least four biomarkers in a multiplexed assay format to simultaneously measure the concentrations of at least four biomarkers in the biological sample, wherein the multiplexed immunoassay comprises: a) combining, in one or more steps: (i) the biological sample; (ii) at least a first antibody to IL-15; a first antibody to CD5; a first antibody to Flt-3L; and a first antibody to salivary amylase, wherein each of the first antibodies is immobilized on separate binding domains; (iii) at least a second antibody to IL-15; a second antibody to CD5; a second antibody to Flt-3L; and a second antibody to salivary amylase, wherein each second antibody is connected to a detectable label; b) forming at least a first, second, third, and fourth binding complex on an at least first, second, third, and fourth binding domains comprising at least IL-15, CD5, Flt-3L, and salivary amylase, and the first and second antibodies for their respective biomarker; c) measuring the concentration of the at least IL-15, CD5, Flt-3L, and salivary amylase on the at least first, second, third, and fourth binding domains.

Biomarkers and Samples

As used herein, the term “biomarker” refers to a biological substance that is indicative of a normal or abnormal process, e.g., disease, infection, or environmental exposure. Biomarkers can be small molecules such as ligands, signaling molecules, or peptides, or macromolecules such as antibodies, receptors, or proteins and protein complexes. A change in the levels of a biomarker can correlate with the risk or progression of a disease or abnormality or with the susceptibility of the disease or abnormality to a given treatment. A biomarker can be useful in the diagnosis of disease risk or the presence of disease in an individual, or to tailor treatments for the disease in an individual (e.g., choices of drug treatment or administration regimes). In evaluating potential drug therapies, a biomarker can be used as a surrogate for a natural endpoint such as survival or irreversible morbidity. If a treatment alters a biomarker that has a direct connection to improved health, the biomarker serves as a “surrogate endpoint” for evaluating clinical benefit. Biomarkers are further described in, e.g., Mayeux, NeuroRx 1(2): 182-188 (2004); Strimbu et al., Curr Opin HIV AIDS 5(6): 463-466 (2010); and Bansal et al., Statist Med 32: 1877-1892 (2013). The term “biomarker,” when used in the context of a specific organism (e.g., human, nonhuman primate or another animal), refers to the biomarker native to that specific organism. For example, “human biomarker” salivary amylase refers to salivary amylase found in humans, i.e., AMY1A, while “nonhuman primate biomarker” salivary amylase refers to salivary amylase found in nonhuman primates, i.e., AMY2A. Unless specified otherwise, the biomarkers referred to in embodiments herein encompass human biomarkers.

As used herein, the term “level” in the context of a biomarker refers to the amount, concentration, or activity of a biomarker. The term “level” can also refer to the rate of change of the amount, concentration, or activity of a biomarker. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a biomarker accumulated in a cell, including, for example, the amount of particular post-synthetic modifications of a biomarker such as a polypeptide, nucleic acid or small molecule. “Level” can also refer to an absolute amount of a biomarker in a sample or to a relative amount of the biomarker, including amount or concentration determined under steady-state or non-steady-state conditions. “Level” can further refer to an assay signal that correlates with the amount, concentration, activity or rate of change of a biomarker. The level of a biomarker can be determined relative to a control marker in a sample. The terms “level” and “concentration” are used interchangeably herein, except when the context clear dictates otherwise.

Biomarkers for assessing radiation exposure can include, e.g., stress and/or damage response markers and damage repair markers. In embodiments, the biomarker for assessing radiation exposure is a DNA damage biomarker. In embodiments, the DNA damage biomarker is p53, p21, ATM serine/threonine kinase (ATM), phosphorylated H2AX histone (γ-H2AX), GADD45A, or combination thereof. Biomarkers for assessing radiation exposure are, in embodiments, not significantly affected by chronic diseases with high prevalence in the human population, such as diabetes, asthma, high blood pressure, heart disease, arthritis and/or other chronic inflammatory or autoimmune diseases. Biomarkers for assessing radiation exposure are, in embodiments, also not affected by other types of trauma (e.g., wounding, burns and/or mental stress) that may also be experienced by individuals in a radiation event.

In embodiments, the biomarker for assessing radiation exposure is an inflammatory response biomarker. An inflammatory response biomarker is a biomarker that is up- or down-regulated during systemic or localized inflammatory response, e.g., caused by radiation exposure. In embodiments, the inflammatory response biomarker is IL-1, IL-2, IL-3, IL-4, IL-5, IL-6, IL-7, IL-10, IL-12, IL-23, TNF-α, INF-γ, C-reactive protein (CRP), serum amyloid A (SAA), CXCL1 (also known as KC/GRO), or combination thereof.

In embodiments, the biomarker for assessing radiation exposure is an acute phase protein. Acute phase proteins (APPs) are a class of proteins whose plasma concentrations increase (positive APPs) or decrease (negative APPs) in response to inflammation, e.g., caused by radiation exposure. See, e.g., Ossetrova et al., Radiat Meas 46(9): 1019-1024 (2011); and Sproull et al., Radiat Res 184(1): 14-23 (2015). In embodiments, the acute phase protein is C-reactive protein (CRP).

In embodiments, the biomarker for assessing radiation exposure is a tissue damage biomarker. A tissue damage biomarker is a biomarker released from a tissue as a result of local tissue damage, e.g., caused by radiation. In embodiments, the tissue damage biomarker is salivary amylase, citrullinated proteins, creatine kinase BB (CKBB), creatine kinase MB (CKMB), creatine kinase MM (CKMM), S100B, surfactant protein D (SP-D), fatty acid binding protein 2 (FABP2), bacterial/permeability-increasing protein (BPI), glial fibrillary acidic protein (GFAP), thrombospondin (TSP), neuron-specific enolase (NSE), cancer antigen 15-3 (CA15-3), or combination thereof.

In embodiments, the biomarker for assessing radiation exposure is a salivary gland damage biomarker. Radiation exposure has been shown to affect salivary gland function (see, e.g., Marmary et al., Cancer Res 76(5): 1170-1180 (2016); Nanduri et al., Radiother Oncol 99(3): 367-372 (2011); Hakim et al., Clin Oral Investig 8(1): 30-35 (2004)). In embodiments, the salivary gland damage biomarker is salivary amylase (AMY1A or AMY2A).

In embodiments, the biomarker for assessing radiation exposure is a tissue damage repair biomarker. A tissue damage repair biomarker is a biomarker that is up- or down-regulated during repair, regeneration, or fibroblastic phase during tissue damage. Tissue damage repair biomarkers can also include proteins associated with soft-tissue repair processes, including but not limited to fibroblast formation, collagen synthesis, and tissue remodeling and realignment. In embodiments, the tissue damage repair biomarker is FMS-like tyrosine kinase 3 ligand (Flt-3L), thyroid peroxidase (TPO), granulocyte-colony stimulating factor (G-CSF), granulocyte-macrophage colony stimulating factor (GM-CSF), keratinocyte growth factor (KGF), stromal cell-derived factor-1 (SDF-1a), erythropoietin (EPO), or combination thereof.

In embodiments, the biomarker for assessing radiation exposure is a hematopoietic repair factor. In embodiments, the biomarker is a hematopoietic cytokine. In embodiments, the biomarker is a hematopoietic progenitor. In embodiments, the biomarker is present on an erythrocyte. In embodiments, the biomarker is present on a platelet. In embodiments, the biomarker is a pro-inflammatory cytokine. In embodiments, the biomarker is present on an innate immune system cell. In embodiments, the hematopoietic repair factor or cytokine is Flt-3L, erythropoietin (EPO), thyroid peroxidase (TPO), IL-12, IL-15, IL-18, or combination thereof.

In embodiments, the biomarker for assessing radiation exposure is a hematology surrogate biomarker. Hematology surrogate biomarkers are generally cell-surface markers on blood cells, which may be used as surrogates to traditional blood cell counts, e.g., for assessing the effect of radiation on specific blood-cell populations. Hematology surrogate biomarkers include markers found on general classes of cells (e.g., leukocytes), or more specific ell types within those classes, such as lymphocytes, neutrophils, platelets, or even more specifically, T-cells or B-cells. In embodiments, the hematology surrogate biomarker is CD5, CD16b, CD20, CD26, CD27, CD40, CD45, CD177, or combination thereof.

In embodiments, the biomarker for assessing radiation exposure is a hematopoietic damage marker. In embodiments, the hematopoietic damage marker is an immune cell surface marker. In embodiments, the hematopoietic damage marker is a T cell surface marker, a B cell surface marker, a lymphocyte surface marker, or a neutrophil surface marker. In embodiments, the hematopoietic damage marker is CD5, CD20, CD27, CD177, or combination thereof.

In embodiments, the method comprises quantifying a combination of the biomarkers described herein in a sample, e.g., a biological sample. In embodiments, the sample is obtained from a subject exposed to radiation, at risk of exposure to radiation, or suspected of having been exposed to radiation exposure. In embodiments, the biomarker combination comprises an inflammatory response biomarker, a tissue damage biomarker, and a tissue damage repair biomarker, a hematology surrogate marker. In embodiments, the biomarker combination comprises a hematopoietic damage marker, a hematopoietic repair factor, a hematopoietic cytokine, and a salivary gland damage marker. In embodiments, the amount of radiation exposure of the subject is determined based on the quantitated amounts of the biomarkers in the combination. In embodiments, quantifying the biomarker combination provides a more accurate and precise determination of the amount of radiation exposure, compared with quantifying each biomarker in the combination individually.

In embodiments, the method comprises quantifying the amounts of at least four biomarkers described herein, in a sample, e.g., a biological sample. In embodiments, the sample is obtained from a subject exposed to radiation, at risk of exposure to radiation, or suspected of having been exposed to radiation exposure. In embodiments, the at least four biomarkers comprise IL-15, CD5, Flt-3L, and salivary amylase. In embodiments, the at least four biomarkers comprise IL-15, CD5, Flt-3L, salivary amylase, and CD20. In embodiments, the at least four biomarkers comprise IL-15, CD5, Flt-3L, salivary amylase, and IL-18. In embodiments, the at least four biomarkers comprise IL-15, CD5, Flt-3L, salivary amylase, and CD27. In embodiments, the at least four biomarkers comprise IL-15, CD5, Flt-3L, salivary amylase, and TPO. In embodiments, the at least four biomarkers comprise IL-15, CD5, Flt-3L, salivary amylase, and one or more of CD20, IL-18, CD27, and TPO. In embodiments, quantifying the amount of at least four biomarkers described herein provides an accurate and precise determination of the amount of radiation exposure. In embodiments, quantifying the amount of at least four biomarkers described herein provides a more accurate and precise determination of the amount of radiation exposure, compared with quantifying less than four of the biomarkers described herein.

In embodiments, the biomarker combination comprises IL-15. IL-15 is a cytokine that regulates activation and proliferation of T and natural killer (NK) cells. In embodiments, IL-15 levels increase in a subject, e.g., a human subject, after radiation exposure. In embodiments, IL-15 levels are higher in a subject exposed to radiation, compared with a subject who has not been exposed to radiation. In embodiments, IL-15 levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises CD5. It was discovered that changes in CD5, which is typically known as a lymphocyte surface marker, can be used to assess radiation exposure in serum and/or plasma samples. It was further discovered that CD5 had relatively normal baseline levels in certain populations that may be subjected to or at risk of radiation exposure, e.g., cancer patients undergoing stem cell transplant (SCT), while baseline levels of biomarkers that have been used to assess radiation exposure, e.g., CD20, vary substantially in these populations. In embodiments, the inclusion of CD5 in the biomarker combination provides improved consistency, redundancy, and accuracy in assessing radiation exposure. In embodiments, CD5 levels decrease in a subject, e.g., a human subject, after radiation exposure. In embodiments, CD5 levels are lower in a subject exposed to radiation, compared with a subject who has not been exposed to radiation. In embodiments, CD5 levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% lower compared with a subject who has not been exposed to radiation.

In embodiments, the biomarker combination comprises Flt-3L. Flt-3L can function as a cytokine and growth factor that increases the number of immune cells (e.g., lymphocytes such as B cells and T cells) by activating hematopoietic progenitors. In embodiments, Flt-3L levels increase in a subject, e.g., a human subject, after radiation exposure. In embodiments, Flt-3L levels are higher in a subject exposed to radiation, compared with a subject who has not been exposed to radiation. In embodiments, Flt-3L levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises salivary amylase. As discussed herein, radiation exposure can damage salivary gland function, and accordingly, salivary amylase levels can be used to assess radiation exposure. In embodiments, salivary amylase levels increase in a subject, e.g., a human subject, after radiation exposure. In embodiments, salivary amylase levels are higher in a subject exposed to radiation, compared with a subject who has not been exposed to radiation. In embodiments, salivary amylase levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises CD20. CD20 is a membrane-embedded surface biomarker that plays a role in the development and differentiation of B cells into plasma cells. In embodiments, CD20 levels decrease in a subject, e.g., a human subject, after radiation exposure. In embodiments, CD20 levels are lower in a subject exposed to radiation, compared with a subject who has not been exposed to radiation. In embodiments, CD20 levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% lower compared with a subject who has not been exposed to radiation.

In embodiments, the biomarker combination comprises IL-18. IL-18 is a proinflammatory cytokine that can modulate innate and adaptive immunity. In embodiments, IL-18 levels increase in a subject, e.g., a human subject, after radiation exposure. In embodiments, IL-18 levels are higher in a subject exposed to radiation, compared with a subject who has not been exposed to radiation. In embodiments, IL-18 levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises CD27. CD27 is a co-stimulatory immune checkpoint molecule. In embodiments, CD27 levels decrease in a subject, e.g., a human subject, after radiation exposure. In embodiments, CD27 levels are lower in a subject exposed to radiation, compared with a subject who has not been exposed to radiation. In embodiments, CD27 levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% lower compared with a subject who has not been exposed to radiation.

In embodiments, the biomarker combination comprises thyroid peroxidase (TPO), also known as thyroperoxidase or iodide peroxidase. In embodiments, TPO levels increase in a subject, e.g., a human subject, after radiation exposure. In embodiments, TPO levels are higher in a subject exposed to radiation, compared with a subject who has not been exposed to radiation. In embodiments, TPO levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises CD177. In embodiments, CD177 levels increase in a subject, e.g., a human subject, after radiation exposure. In embodiments, CD177 levels are higher in a subject exposed to radiation, compared with a subject who has not been exposed to radiation. In embodiments, CD177 levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises erythropoietin (EPO). In embodiments, EPO levels increase in a subject, e.g., a human subject, after radiation exposure. In embodiments, EPO levels are higher in a subject exposed to radiation, compared with a subject who has not been exposed to radiation. In embodiments, EPO levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises IL-12. In embodiments, IL-12 levels increase in a subject, e.g., a human subject, after radiation exposure. In embodiments, IL-12 levels are higher in a subject exposed to radiation, compared with a subject who has not been exposed to radiation. In embodiments, IL-18 levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, the biomarker combination comprises C-reactive protein (CRP). In embodiments, CRP levels increase in a subject, e.g., a human subject, after radiation exposure. In embodiments, CRP levels are higher in a subject exposed to radiation, compared with a subject who has not been exposed to radiation. In embodiments, CRP levels in a subject exposed to radiation are about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 250%, about 500%, or more than 500% higher compared with a subject who has not been exposed to radiation.

In embodiments, changes in a subject's biomarker levels are observable (e.g., increase or decrease in the manner described herein) within about 10 minutes to about 1 year, about 30 minutes to about 6 months, about 1 hour to about 1 month, about 12 hours to about 2 weeks, about 1 day to about 7 days, about 2 days to about 6 days, or about 3 days to about 4 days after the subject is exposed to radiation. In embodiments, changes in a subject's biomarker levels are observable about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 2 weeks, about 1 month, about 3 months, about 6 months, or about 1 year after a subject is exposed to radiation. Different biomarkers in the same subject may have varying magnitude of change in response to radiation, for example, depending on whether the biomarker is an acute response biomarker or a biomarker related to a long-term effect. Thus, a multi-biomarker assay for a combination of biomarkers should consider the response timing of each of the biomarkers.

In embodiments, changes in a subject's biomarker levels are observable (e.g., increase or decrease in the manner described herein) when the subject has been exposed to about 0.5 Gy to about 10 Gy, about 1.0 Gy to about 9.0 Gy, about 1.5 Gy to about 8.5 Gy, about 2.0 Gy to about 8.0 Gy, about 2.5 to about 7.5 Gy, about 3.0 Gy to about 7.0 Gy, about 3.5 Gy to about 6.5 Gy, about 4.0 Gy to about 6.0 Gy, or about 4.5 Gy to about 5.5 Gy of radiation. In embodiments, changes in a subject's biomarker levels are observable when the subject has been exposed to about 0.1 Gy, about 0.2 Gy, about 0.3 Gy, about 0.4 Gy, about 0.5 Gy, about 0.6 Gy, about 0.7 Gy, about 0.8 Gy, about 0.9 Gy, about 1.0 Gy, about 1.5 Gy, about 2.0 Gy, about 2.5 Gy, about 3.5 Gy, about 4.0 Gy, about 4.5 Gy, about 5.0 Gy, about 5.5 Gy, about 6.0 Gy, about 6.5 Gy, about 7.0 Gy, about 7.5 Gy, about 8.0 Gy, about 8.5 Gy, about 9.0 Gy, about 9.5 Gy, or about 10 Gy of radiation. In embodiments, the present method comprising quantifying a combination of biomarkers is capable of accurately determining low dose radiation exposure (e.g., less than 1 Gy). In embodiments, the present method comprising quantifying a combination of biomarkers is capable of accurately determining moderate dose radiation exposure (e.g., about 1 Gy to about 3 Gy). In embodiments, the present method comprising quantifying a combination of biomarkers is capable of accurately determining high dose radiation exposure (e.g., higher than about 2 Gy or higher than about 3 Gy). In embodiments, doses at or above 2 Gy signify the triage threshold requiring treatment. In embodiments, a method comprising quantifying a combination of the biomarkers provided herein has improved sensitivity in determining low dose radiation compared with methods in which only a single biomarker is quantified. In embodiments, a method comprising quantifying a combination of the biomarkers provided herein has improved dynamic range in determining radiation exposure compared with methods in which a single biomarker is quantified.

In embodiments, the biomarkers described herein are measured in a sample, e.g., a biological sample. In embodiments, the sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions, or an extraction, purification therefrom, or dilution thereof. Further exemplary biological samples include but are not limited to physiological samples, samples containing suspensions of cells such as mucosal swabs, tissue aspirates, tissue homogenates, cell cultures, and cell culture supernatants. In embodiments, the biological sample is whole blood, serum, plasma, cerebrospinal fluid, urine, saliva, or an extraction or purification therefrom, or dilution thereof. In embodiments, the biological sample is serum or plasma. In embodiments, the plasma is in EDTA, heparin, or citrate.

In embodiments, the sample is obtained from an individual, e.g., a human. In embodiments, the sample comprises a plasma (e.g., in EDTA, heparin, or citrate) sample from an individual. In embodiments, the sample comprise a serum sample from an individual. In embodiments, the sample is obtained from a healthy individual. In embodiments, the sample is obtained from an individual not exposed to radiation. In embodiments, the sample is obtained from an individual exposed to radiation, at risk of exposure to radiation, or suspected of having been exposed to radiation. In embodiments, the sample is obtained from an individual exposed to a known dose of radiation. In embodiments, the sample is obtained from an individual having or at risk of disease, e.g., as a result of exposure to radiation. In embodiments, the sample is obtained from a patient exposed to radiation as part of a treatment. In embodiments, the patient is undergoing or has undergone stem cell transplant (SCT) therapy. In embodiments, the biological sample is obtained from an individual within about 1 to about 7 days, e.g., 1, 2, 3, 4, 5, 6, or 7 days, of exposure or suspected exposure to radiation.

Samples may be obtained from a single source described herein, or may contain a mixture from two or more sources, e.g., pooled from one or more individuals who may have been exposed to radiation in a similar manner.

Assay Methods and Components

Levels of the biomarkers described herein can be measured using a number of techniques available to a person of ordinary skill in the art, e.g., direct physical measurements (e.g., mass spectrometry) or binding assays (e.g., immunoassays, agglutination assays and immunochromatographic assays). Biomarkers identified herein can be measured by any suitable immunoassay method, including but not limited to, ELISA, microsphere-based immunoassay methods, lateral flow test strips, antibody based dot blots or western blots. The method can also comprise measuring a signal that results from a chemical reactions, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques can detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques can be used that do not require the use of labels, e.g., techniques based on measuring mass (e.g., surface acoustic wave measurements), refractive index (e.g., surface plasmon resonance measurements), or the inherent luminescence of a biomarker.

Binding assays for measuring biomarker levels can use solid phase or homogenous formats. Suitable assay methods include sandwich or competitive binding assays. Examples of sandwich immunoassays are described in U.S. Pat. Nos. 4,168,146 and 4,366,241. Examples of competitive immunoassays include those disclosed in U.S. Pat. Nos. 4,235,601, 4,442,204, and 5,208,535.

Multiple biomarkers can be measured using a multiplexed assay format, e.g., multiplexing through the use of binding reagent arrays, multiplexing using spectral discrimination of labels, multiplexing of flow cytometric analysis of binding assays carried out on particles, e.g., using the LUMINEX® system. Suitable multiplexing methods include array based binding assays using patterned arrays of immobilized antibodies directed against the biomarkers of interest. Various approaches for conducting multiplexed assays have been described (see, e.g., US 2003/0113713; US 2003/0207290; US 2004/0022677; US 2004/0189311; US 2005/0052646; US 2005/0142033; US 2006/0069872; U.S. Pat. Nos. 6,977,722; 7,842,246; 10,189,023; and 10,201,812). One approach to multiplexing binding assays involves the use of patterned arrays of binding reagents, e.g., as described in U.S. Pat. Nos. 5,807,522 and 6,110,426; Delehanty, “Printing functional protein microarrays using piezoelectric capillaries,” Methods Mol Bio 278: 135-144 (2004); Lue et al., “Site-specific immobilization of biotinylated proteins for protein microarray analysis,” Methods Mol Biol 278: 85-100 (2004); Lovett, “Toxicogenomics: Toxicologists Brace for Genomics Revolution,” Science 289: 536-537 (2000); Berns, “Cancer: Gene expression in diagnosis,” Nature 403: 491-492 (2000); Walt, “Molecular Biology: Bead-based Fiber-Optic Arrays,” Science 287: 451-452 (2000). Another approach involves the use of binding reagents coated on beads that can be individually identified and interrogated. See, e.g., WO 99/26067, which describes the use of magnetic particles that vary in size to assay multiple analytes; particles belonging to different distinct size ranges are used to assay different analytes. The particles are designed to be distinguished and individually interrogated by flow cytometry. Vignali, “Multiplexed Particle-Based Flow Cytometric Assays,” J Immunol Meth 243: 243-255 (2000) has described a multiplex binding assay in which 64 different bead sets of microparticles are employed, each having a uniform and distinct proportion of two. A similar approach involving a set of 15 different beads of differing size and fluorescence has been disclosed as useful for simultaneous typing of multiple pneumococcal serotypes (Park et al., “A Latex Bead-Based Flow Cytometric Immunoassay Capable of Simultaneous Typing of Multiple Pneumococcal Serotypes (Multibead Assay),” Clin Diag Lab Immunol 7: 4869 (2000)). Bishop et al. have described a multiplex sandwich assay for simultaneous quantification of six human cytokines (Bishop et al., “Simultaneous Quantification of Six Human Cytokines in a Single Sample Using Microparticle-based Flow Cytometric Technology,” Clin Chem 45:1693-1694 (1999)).

A diagnostic test can be conducted in a single assay chamber, such as a single well of an assay plate or an assay chamber that is an assay chamber of a cartridge. The assay modules, e.g., assay plates or cartridges or multi-well assay plates, methods and apparatuses for conducting assay measurements suitable for the present invention, are described, e.g., in US 2004/0022677; US 2004/0189311; US 2005/0052646; and US 2005/0142033. Assay plates and plate readers are commercially available (MULTI-SPOT® and MULTI-ARRAY® plates and SECTOR® instruments, MESO SCALE DISCOVERY®, a division of Meso Scale Diagnostics, LLC, Rockville, Md.).

In embodiments, the present disclosure provides a multiplexed immunoassay method comprising quantifying the amounts of at least four biomarkers in a biological sample. In embodiments, the quantifying comprises measuring the concentrations of the at least four biomarkers in a multiplexed assay format. In embodiments, the concentrations of the at least four biomarkers are measured simultaneously. In embodiments, the concentrations of the at least four biomarkers are measured sequentially. In embodiments, the multiplexed immunoassay comprises combining, in one or more steps, the biological sample and at least a first, second, third and fourth binding reagent, wherein the first, second, third and fourth binding reagent is a binding partner of each of the four biomarkers.

Binding

In embodiments, the biological sample is combined with the first, second, third, and fourth binding reagents simultaneously. In embodiments, the biological sample is combined with the first, second, third, and fourth binding reagents sequentially. In embodiments, the first, second, third, and fourth binding reagents are pre-mixed, and the combining comprises contacting the biological sample with the pre-mixed binding reagent mixture.

In embodiments, the binding reagent is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody. In embodiments, the binding reagent for IL-15 is a first antibody to IL-15; the binding reagent for CD5 is a first antibody to CD5; the binding reagent for Flt-3L is a first antibody to Flt-3L; and the binding reagent for salivary amylase is a first antibody to amylase.

In embodiments, the first binding reagent and a first biomarker of the at least four biomarkers form a first binding complex. In embodiments, the second binding reagent and a second biomarker of the at least four biomarkers form a second binding complex. In embodiments, the third binding reagent and a third biomarker of the at least four biomarkers form a third binding complex. In embodiments, the fourth binding reagent and a fourth biomarker of the at least four biomarkers form a fourth binding complex. In embodiments, the at least four biomarkers comprise IL-15, CD5, Flt-3L, and salivary amylase. In embodiments, the first binding reagent is a binding partner of IL-15, the second binding reagent is a binding partner of CD5, the third binding reagent is a binding partner of Flt-3L, and the fourth binding reagent is a binding partner of salivary amylase. In embodiments, the first binding reagent and IL-15 form a first binding complex, the second binding reagent and CD5 form a second binding complex, the third binding reagent and Flt-3L form a third binding complex, and the fourth binding reagent and salivary amylase form a fourth binding complex.

In embodiments, each of the binding reagents are immobilized on separate binding domains. In embodiments, the first, second, third, and fourth binding reagents are immobilized on associated first, second, third, and fourth binding domains. In embodiments, each binding domain comprises a targeting agent capable of binding to a targeting agent complement, wherein the targeting agent complement is connected to a linking agent, and each binding reagent comprises a supplemental linking agent capable of binding to the linking agent. Thus, in embodiments, the binding reagent is immobilized on the binding domain by: (1) binding each binding reagent to the targeting agent complement via the supplemental linking agent and the linking agent; and (2) binding each product of step (1) to a binding domain comprising the targeting agent, wherein (i) each binding domain comprises a different targeting agent, and (ii) each targeting agent selectively binds to one of the targeting agent complements, thereby immobilizing each binding reagent to its associated binding domain.

In embodiments, the first, second, third, and fourth binding domains respectively comprise first, second, third, and fourth targeting agents. In embodiments, the first, second, third, and fourth targeting agents are respective binding partners of first, second, third, and fourth targeting agent complements. In embodiments, the first, second, third, and fourth targeting agent complements are each connected to a linking agent. In embodiments, the first, second, third, and fourth binding reagents each comprise a supplemental linking agent. In embodiments, the first, second, third, and fourth binding reagents bind to the first, second, third, and fourth targeting agent complements, respectively, via the supplemental linking agent on each of the binding reagents and the linking agent connected to each of the targeting agent complements. In embodiments, the first, second, third, and fourth binding reagents, each bound to its respective targeting agent complement, are contacted with the first, second, third and fourth binding domains and bind to first, second, third, and fourth targeting agents, respectively, via the targeting agent complement on each of the binding reagent and the targeting agent on each of the binding domains.

In embodiments, an optional bridging agent, which is a binding partner of both the linking agent and the supplemental linking agent, bridges the linking agent and supplemental linking agent, such that the first, second, third, and fourth binding reagents, each bound to its respective targeting reagent complement, are contacted with the first, second, third and fourth binding domains and bind to first, second, third, and fourth targeting agents, respectively, via the bridging agent, the targeting agent complement on each of the binding reagent, and the targeting agent on each of the binding domains.

In embodiments, the targeting agent and targeting agent complement are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin.

In embodiments, the linking agent and supplemental linking agent are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the linking agent and supplemental linking agent are cross-reactive moieties, e.g., thiol and maleimide or iodoacetamide; aldehyde and hydrazide; or azide and alkyne or cycloalkyne. In embodiments, the linking agent is avidin or streptavidin, and the supplemental linking agent is biotin. In embodiments, the targeting agent and targeting agent complement are complementary oligonucleotides. In embodiments, the targeting agent complement is streptavidin, the targeting agent is biotin, and the linking agent and the supplemental linking agent are complementary oligonucleotides.

In embodiments that include the optional bridging agent, the bridging agent is streptavidin or avidin, and the linking agents and the supplemental linking agents are each biotin.

In embodiments, each binding domain is an element of an array of binding elements. In embodiments, the binding domains are on a surface. In embodiments, the surface is a plate. In embodiments, the surface is a well in a multi-well plate. In embodiments, the array of binding elements is located within a well of a multi-well plate. In embodiments, the surface is a particle. In embodiments, each binding domain is positioned on one or more particles. In embodiments, the particles are in a particle array. In embodiments, the particles are coded to allow for identification of specific particles and distinguish between each binding domain.

Detection

In embodiments, the multiplexed immunoassay method further comprises detecting the binding complexes comprising each binding reagent and its target biomarker. In embodiments, each binding complex comprising a binding reagent and its target biomarker further comprises a detection reagent. In embodiments, the detection reagent is an antibody, antigen, ligand, receptor, oligonucleotide, hapten, epitope, mimotope, or aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, detection reagent comprises at least one heavy or light chain complementarity determining region (CDR) of an antibody. In embodiments, the detection reagent comprises at least two CDRs from one or more antibodies. In embodiments, the detection reagent is an antibody.

In embodiments, the components of the combining step in the multiplexed immunoassay method further comprise at least a first, second, third, and fourth detection reagent that each bind a biomarker, and the binding complexes further comprise the at least first, second, third, and fourth detection reagents. Thus, in embodiments, the first binding complex in the first binding domain comprises the first biomarker, the first binding reagent, and the first detection reagent; the second binding complex in the second binding domain comprises the second biomarker, second first binding reagent, and the second detection reagent; the third binding complex in the third binding domain comprises the third biomarker, the third binding reagent, and the third detection reagent; and the fourth binding complex in the fourth binding domain comprises the fourth biomarker, the fourth binding reagent, and the fourth detection reagent.

In embodiments, the at least four biomarkers comprise IL-15, CD5, Flt-3L, and salivary amylase. In embodiments, the first binding complex comprises IL-15 and its binding and detection reagents, the second binding complex comprises CD5 and its binding and detection reagents, the third binding complex comprises Flt-3L and its binding and detection reagents, and the fourth binding complex comprises salivary amylase and its binding and detection reagents. In embodiments, the first, second, third, and fourth binding complexes are immobilized on the first, second, third, and fourth binding domains in the manner described herein.

In embodiments, the binding reagent for IL-15 is a first antibody to IL-15; the binding reagent for CD5 is a first antibody to CD5; the binding reagent for Flt-3L is a first antibody to Flt-3L; and the binding reagent for salivary amylase is a first antibody to amylase. In embodiments, the detection reagent is an antibody. In embodiments, the detection reagent for IL-15 is a second antibody to IL-15; the detection reagent for CD5 is a second antibody to CD5; the detection reagent for Flt-3L is a second antibody to Flt-3L; and the detection reagent for salivary amylase is a second antibody to amylase. Accordingly, in embodiments, the first binding complex in the first binding domain comprises IL-15 and its first and second antibodies; the second binding complex in the second binding domain comprises CD5 and its first and second antibodies; the third binding complex in the third binding domain comprises Flt-3L and its first and second antibodies; and the fourth binding complex in the fourth binding domain comprises salivary amylase and its first and second antibodies.

In embodiments, the detection reagent comprises a detectable label. In embodiments, measuring the concentration of the biomarkers in each of the binding complexes comprises measuring the presence and/or amount of the detectable label. In embodiments, the detectable label is measured by light scattering, optical absorbance, fluorescence, chemiluminescence, electrochemiluminescence (ECL), bioluminescence, phosphorescence, radioactivity, magnetic field, or combination thereof. In embodiments, the detectable label comprises an electrochemiluminescence label. In embodiments, the detectable label comprises ruthenium. In embodiments, measuring the concentration of the biomarkers comprises measuring the presence and/or amount of the detectable label by electrochemiluminescence. In embodiments, the measuring of the detectable label comprises measuring an electrochemiluminescence signal.

In embodiments, the surface comprising the binding domains described herein comprises an electrode. In embodiments, the electrode is a carbon ink electrode. In embodiments, the measuring of the detectable label comprises applying an electrode and measuring electrochemiluminescence. In embodiments, applying a potential to the electrode generates an electrochemiluminescence signal. In embodiments, the strength of the electrochemiluminescence signal is based on the biomarker concentration in the binding complex.

In embodiments, a method for performing the multiplexed immunoassays described herein includes:

1. Coupling the supplemental linking agent to the binding reagent. In embodiments, the supplemental linking agent is biotin, and the binding reagent is an antibody. Methods of biotinylating proteins, e.g., antibodies, are known to the skilled artisan. The coupling may include agitation, e.g., vortexing or shaking, and incubation, e.g., for about 10 minutes to about 2 hours, about 20 minutes to about 1 hour, or about 30 minutes. After the incubation, the coupling reaction can be stopped by adding a stop solution followed by agitation (e.g., vortex), and incubation for about 10 minutes to about 2 hours, about 20 minutes to 1 hour, or about 30 minutes. In embodiments, the stop solution comprises a reagent that inactivates one or more reagents in the coupling reaction. In embodiments, the coupling further comprises contacting the binding reagent comprising the supplemental linking agent with a linking agent connected to a targeting agent complement or with a bridging agent linked to a linking agent connected to a targeting agent complement. In embodiments, each unique binding reagent is contacted with a linking agent connected to a unique targeting agent complement. In embodiment, the targeting agent complement is an oligonucleotide.

2. Mixing binding reagents for each of the biomarkers in a solution. In embodiments, the mixture of binding reagents comprises a first, second, third, and fourth binding reagents for binding to IL-15, CD5, Flt-3L, and salivary amylase, respectively. In embodiments, the mixture of binding reagents further comprises at least one additional binding reagent for binding to one or more of CD20, IL-18, CD27, and TPO. In embodiments, the mixture of binding reagents comprises CD20, IL-15, AMY1A, CD5, and Flt-3L.

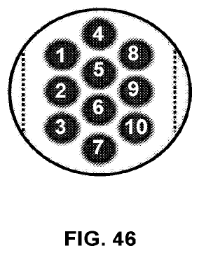

3. Coating the binding domains with the mixture of binding reagents. In embodiments, the binding domains are arranged on a surface. In embodiments, the surface is a well of a multi-well plate. In embodiments, the multi-well plate is a 96-well assay plate. In embodiments, each well comprises ten distinct binding domains. In embodiments, the mixture of binding reagents is added to the well. In embodiments, each binding domain comprises a targeting agent for one of the unique targeting agent complements. In embodiments, the targeting agent is a complementary oligonucleotide of the targeting agent complement. In embodiments, the mixture of binding reagents is added to the well and incubated for about 10 minutes to about 4 hours, about 30 minutes to about 2 hours, or about 1 hour. In embodiments, the incubation is at 20° C. to about 30° C., about 22° C. to about 28° C., or about 24° C. to about 26° C. In embodiments, the incubation is performed with agitation, e.g., shaking. In embodiments, the surface comprising the binding domains, e.g., the plate, is washed after incubation to remove excess binding reagent. An embodiment of a well in a 96-well assay plate, comprising ten binding domains (“spots”), is shown in FIG. 46. In embodiments, the binding reagent for CD20 is immobilized in Spot 1 of FIG. 46, the binding reagent for IL-15 is immobilized in Spot 2 of FIG. 46, the binding reagent for AMY1A is immobilized in Spot 3 of FIG. 46, the binding reagent for CD5 is immobilized in Spot 6 of FIG. 46, the binding reagent for Flt-3L is immobilized in Spot 10 of FIG. 46, and Spots 4, 5, 7, 8, and 9 of FIG. 46 do not comprise a specific binding reagent, and in embodiments, each comprises an immobilized BSA.

4. Contacting the surface comprising the binding domains with the detection reagent for each biomarker and the sample comprising the biomarkers, calibration reagent, or control reagent. In embodiments, the detection reagents are added before or after the other assay components. In embodiments, the detection reagent is added at a volume of about 10 μL to about 50 μL, about 20 μL to about 30 μL, or about 25 μL. In embodiments, the sample, calibration reagent, or control reagent is added at a volume of about 10 μL to about 50 μL, about 20 μL to about 30 μL, or about 25 μL. In embodiments, the volume of the detection reagent and sample, calibration reagent, or control reagent is such that the final assay reaction volume is about 50 μL. In embodiments, the assay reactions are incubated for about 10 minutes to about 4 hours, about 30 minutes to about 2 hours, or about 1 hour. In embodiments, the incubation is at 20° C. to about 30° C., about 22° C. to about 28° C., or about 24° C. to about 26° C. In embodiments, the incubation is performed with agitation, e.g., shaking. In embodiments, the surface comprising the binding domains, e.g., the plate, is washed after incubation to remove excess detection reagent and unbound components of the sample.

5. Adding read buffer and reading the assay immediately. In embodiments, the read buffer comprises an ECL co-reactant. In embodiments, the read buffer is 2×MSD Read Buffer T. In embodiments, the read buffer is a read buffer provided in, e.g., U.S. Provisional Application No. 62/787,892, filed on Jan. 3, 2019. In embodiments, the read buffer is added at a volume of about 50 μL to about 200 μL, about 100 μL to about 180 μL, or about 150 μL.

Assay Desensitization

As discussed herein, one particular challenge of developing the multiplexed assay is finding biomarkers effective for a determining a particular condition that have levels within the assay's dynamic range at the same dilution. The inventors discovered that such was not the case for AMY1A. AMY1A was much more abundant in samples than the other biomarkers. To overcome this problem, the inventors used excess, unlabeled, non-immobilized binding reagents for AMY1A to decrease the amount of AMY1A signal produced in the multiplexed assay.

Thus, it was discovered that this non-immobilized competing reagent can be included in the assay for a high abundance biomarker to effectively lower the concentration of that biomarker available for binding to the immobilized binding reagent. In embodiments, the biomarker that binds to the non-immobilized competing reagent does not form a complex with a binding reagent and is consequently not available for subsequent detection and/or quantitation. Thus, in embodiments, a non-immobilized competing reagent is added to compete with an immobilized binding reagent for binding to its target biomarker, thereby desensitizing the measurement of that biomarker. As used herein, a “desensitized” measurement or “desensitized” assay means that the amount of biomarker available for binding to the immobilized binding reagent and detection by the detection reagent is reduced by the presence of the non-immobilized competing reagent. In general, an assay is desensitized to achieve a desired dynamic range.

In embodiments, the non-immobilized competing reagent is an antibody, antigen, ligand, receptor, oligonucleotide, hapten, epitope, mimotope, or aptamer. In embodiments, the non-immobilized competing reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the non-immobilized competing reagent comprises at least one heavy or light chain complementarity determining region (CDR) of an antibody. In embodiments, the non-immobilized competing reagent comprises at least two CDRs from one or more antibodies. In embodiments, the non-immobilized competing reagent is an antibody.

In embodiments, the non-immobilized competing reagent is substantially the same substance as the binding reagent, except the non-immobilized competing reagent is not immobilized to the binding domain. In embodiments, the non-immobilized competing reagent is substantially the same substance as the binding reagent, except the non-immobilized competing reagent does not comprise a supplemental linking domain for attachment to the binding domain. In embodiments, the non-immobilized competing reagent has substantially the same binding capability for the biomarker as the binding reagent. In embodiments, the non-immobilized competing reagent has substantially the same specificity for the biomarker as the binding reagent. In embodiments, the non-immobilized competing reagent is a different substance than the binding reagent and with substantially the same binding capability and/or specificity for the biomarker as the binding reagent. As used herein, the term “substantially” is within a range that the skilled artisan would understand to be functionally or biochemically equivalent. For example, two substances with “substantially the same” binding ability or specificity to a given biomarker can differ in their binding ability or specificity by about 1%, about 2%, about 3%, about 4%, about 5%, about 6%, about 7%, about 8%, about 9%, about 10%, about 15%, or about 20%, so long as the desired effect (e.g., assay desensitization) is achieved.

In embodiments, the non-immobilized competing reagent is substantially the same substance as the detection reagent, except the non-immobilized competing reagent does not comprise a detectable label. In embodiments, the non-immobilized competing reagent has substantially the same binding capability for the biomarker as the detection reagent. In embodiments, the non-immobilized competing reagent has substantially the same specificity for the biomarker as the detection reagent. In embodiments, the non-immobilized competing reagent is a different substance than the detection reagent and with substantially the same binding capability and/or specificity for the biomarker as the detection reagent.

In embodiments, the non-immobilized competing reagent is capable of binding one of the at least four biomarkers in the sample. In embodiments, the non-immobilized competing reagent competes with the first, second, third, or fourth binding reagent for binding to its target biomarker. In embodiments, the non-immobilized competing reagent competes with the first, second, third, or fourth detection reagent for binding to its target biomarker. In embodiments, one or more of the at least four biomarkers is present in the sample at a higher abundance compared with the remaining biomarkers. In embodiments, the at least four biomarkers comprise IL-15, CD5, Flt-3L, and salivary amylase. In embodiments, the non-immobilized competing reagent competes with the first binding reagent for binding to IL-15. In embodiments, the non-immobilized competing reagent competes with the first detection reagent for binding to IL-15. In embodiments, the non-immobilized competing reagent competes with the second binding reagent for binding to CD5. In embodiments, the non-immobilized competing reagent competes with the second detection reagent for binding to CD5. In embodiments, the non-immobilized competing reagent competes with the third binding reagent for binding to Flt-3L. In embodiments, the non-immobilized competing reagent competes with the third detection reagent for binding to Flt-3L. Typically, salivary amylase is present in a sample at a higher concentration compared with IL-15, CD5, and Flt-3L. In embodiments, the non-immobilized competing reagent competes with the fourth binding reagent for binding to salivary amylase. In embodiments, the non-immobilized competing reagent competes with the fourth detection reagent for binding to salivary amylase.

In embodiments where the binding reagent and detection reagent are antibodies as described herein, the non-immobilized competing reagent competes with the first antibody for binding to IL-15. In embodiments where the binding reagent and detection reagent are antibodies as described herein, the non-immobilized competing reagent competes with the second antibody for binding to IL-15. In embodiments, the non-immobilized competing reagent competes with the first antibody for binding to CD5. In embodiments, the non-immobilized competing reagent competes with the second antibody for binding to CD5. In embodiments, the non-immobilized competing reagent competes with the first antibody for binding to Flt-3L. In embodiments, the non-immobilized competing reagent competes with the second antibody for binding to Flt-3L. In embodiments, the non-immobilized competing reagent competes with the first antibody for binding to salivary amylase. In embodiments, the non-immobilized competing reagent competes with the second antibody for binding to salivary amylase.

Additional Biomarkers

In embodiments, quantifying the amounts of more than four biomarkers in a sample provides an improved assessment of radiation exposure. In embodiments, quantifying the amounts of more than four biomarkers in a sample increases accuracy of the assessment for radiation exposure. In embodiments, quantifying the amounts of more than four biomarkers in a sample increases sensitivity of the measurement. Improvements gained with additional biomarkers in the multiplexed immunoassay should be evaluated against the potential increase in difficulty of performing the assay, e.g., as described herein. In embodiments, the method comprises quantifying at least four biomarkers. In embodiments, the method comprises quantifying at least five biomarkers. In embodiments, the method comprises quantifying at least six biomarkers.

In embodiments, the method further comprises measuring, in the multiplexed assay format, at least one additional biomarker in the biological sample, wherein the at least one additional biomarker is CD20, IL-18, CD27, thyroid peroxidase (TPO), or combination thereof. Thus, in embodiments, the multiplexed immunoassay further includes combining, with the biological sample, one or more additional binding reagents for the at least one additional biomarker. In embodiments, the one or more additional binding reagents are binding partners of CD20, IL-18, CD27, and/or TPO and form additional binding complexes on additional binding domains. In embodiments, the additional binding complexes further comprise one or more additional detection reagents for the at least one additional biomarker. In embodiments, the one or more additional detection reagents bind to CD20, IL-18, CD27, and/or TPO. In embodiments, the method further comprises measuring the concentrations of the additional biomarkers in each of the additional binding complexes. In embodiments, the method further comprises measuring the concentrations of CD20, IL-18, CD27, and/or TPO. In embodiments, the multiplexed immunoassay further includes combining, with the biological sample, one or more additional first and second antibodies for the at least one additional biomarker. In embodiments, the one or more additional first and second antibodies bind to CD20, IL-18, CD27, and/or TPO.

Thus, in embodiments, the method comprises simultaneously measuring the concentrations of IL-15; CD5; Flt-3L; salivary amylase; and one or more of: CD20, IL-18, CD27, and TPO, in the biological sample. In embodiments, the method comprises simultaneously measuring the concentrations of IL-15, CD5, Flt-3L, salivary amylase, and CD20 in the biological sample. In embodiments, the method comprises simultaneously measuring the concentrations of IL-15, CD5, Flt-3L, salivary amylase, and IL-18 in the biological sample. In embodiments, the method comprises simultaneously measuring the concentrations of IL-15, CD5, Flt-3L, salivary amylase, and CD27 in the biological sample. In embodiments, the method comprises simultaneously measuring the concentrations of IL-15, CD5, Flt-3L, salivary amylase, and TPO in the biological sample.

In embodiments, the method comprises simultaneously measuring the concentrations of IL-15, CD5, Flt-3L, salivary amylase, CD20, and IL-18 in the biological sample. In embodiments, the method comprises simultaneously measuring the concentrations of IL-15, CD5, Flt-3L, salivary amylase, CD20, and CD27 in the biological sample. In embodiments, the method comprises simultaneously measuring the concentrations of IL-15, CD5, Flt-3L, salivary amylase, CD20, and TPO in the biological sample. In embodiments, the method comprises simultaneously measuring the concentrations of IL-15, CD5, Flt-3L, salivary amylase, IL-18, and CD27 in the biological sample. In embodiments, the method comprises simultaneously measuring the concentrations of IL-15, CD5, Flt-3L, salivary amylase, IL-18, and TPO in the biological sample. In embodiments, the method comprises simultaneously measuring the concentrations of IL-15, CD5, Flt-3L, salivary amylase, CD27, and TPO in the biological sample.

In embodiments, the method comprises simultaneously measuring the concentrations of IL-15, CD5, Flt-3L, salivary amylase, CD20, IL-18, and CD27 in the biological sample. In embodiments, the method comprises simultaneously measuring the concentrations of IL-15, CD5, Flt-3L, salivary amylase, CD20, IL-18, and TPO in the biological sample. In embodiments, the method comprises simultaneously measuring the concentrations of IL-15, CD5, Flt-3L, salivary amylase, CD20, CD27, and TPO in the biological sample. In embodiments, the method comprises simultaneously measuring the concentrations of IL-15, CD5, Flt-3L, salivary amylase, IL-18, CD27, and TPO in the biological sample. In embodiments, the method comprises simultaneously measuring the concentrations of IL-15, CD5, Flt-3L, salivary amylase, CD20, IL-18, CD27, and TPO in the biological sample.

The concentration of various reagents used in the assays described herein may be selected during assay optimization. The skilled artisan will understand that the concentrations of each reagent should be selected for each biomarker, such that the binding reagent is capable of binding all of the biomarker in the sample; the detection reagent is capable of binding all of the biomarker in the binding complex on the binding domain; and the non-immobilized, competing reagent is capable of effectively desensitizing the assay for the biomarker. In embodiments, the concentration of each binding reagent in the solution used to coat (i.e., coating solution) the binding domain is about 0.05 μg/mL to about 5 μg/mL; about 0.1 μg/mL to about 1 μg/mL; about 0.2 μg/mL to about 0.5 μg/mL; or about 0.25 to about 0.3 μg/mL. In embodiments, the concentration of each binding reagent in the solution used to coat the binding domain is about 0.1 μg/mL, about 0.11 μg/mL, about 0.12 μg/mL, about 0.13 μg/mL, about 0.14 μg/mL, about 0.15 μg/mL, about 0.16 μg/mL, about 0.17 μg/mL, about 0.18 μg/mL, about 0.19 μg/mL, about 0.2 μg/mL, about 0.21 μg/mL, about 0.22 μg/mL, about 0.23 μg/mL, about 0.24 μg/mL, about 0.25 μg/mL, about 0.26 μg/mL, about 0.27 μg/mL, about 0.28 μg/mL, about 0.29 μg/mL, about 0.3 μg/mL, about 0.31 μg/mL, about 0.32 μg/mL, about 0.33 μg/mL, about 0.34 μg/mL, about 0.35 μg/mL, about 0.36 μg/mL, about 0.37 μg/mL, about 0.38 μg/mL, about 0.39 μg/mL, about 0.4 μg/mL, about 0.5 μg/mL, about 0.6 μg/mL, about 0.7 μg/mL, about 0.8 μg/mL, about 0.9 μg/mL, or about 1 μg/mL.

In embodiments, the concentration of the binding reagent for IL-15 in the coating solution is about 0.1 μg/mL to about 1 μg/mL, about 0.2 μg/mL to about 0.5 μg/mL, about 0.25 μg/mL to about 0.3 μg/mL, or about 0.285 μg/mL. In embodiments, the concentration of the binding reagent for CD5 in the coating solution is about 0.1 μg/mL to about 1 μg/mL, about 0.2 μg/mL to about 0.5 μg/mL, about 0.25 μg/mL to about 0.3 μg/mL, or about 0.285 μg/mL. In embodiments, the concentration of the binding reagent for Flt-3L in the coating solution is about 0.1 μg/mL to about 1 μg/mL, about 0.2 μg/mL to about 0.5 μg/mL, about 0.25 μg/mL to about 0.3 μg/mL, or about 0.285 μg/mL. In embodiments, the concentration of the binding reagent for salivary amylase in the coating solution is about 0.1 μg/mL to about 1 μg/mL, about 0.2 μg/mL to about 0.5 μg/mL, about 0.25 μg/mL to about 0.3 μg/mL, or about 0.285 μg/mL. In embodiments, the concentration of the binding reagent for CD20 in the coating solution is about 0.1 μg/mL to about 1 μg/mL, about 0.2 μg/mL to about 0.5 μg/mL, about 0.25 μg/mL to about 0.3 μg/mL, or about 0.285 μg/mL. In embodiments, the concentration of the binding reagent for IL-18 in the coating solution is about 0.1 μg/mL to about 1 μg/mL, about 0.2 μg/mL to about 0.5 μg/mL, about 0.25 μg/mL to about 0.3 μg/mL, or about 0.285 μg/mL. In embodiments, the concentration of the binding reagent for CD27 in the coating solution is about 0.1 μg/mL to about 1 μg/mL, about 0.2 μg/mL to about 0.5 μg/mL, about 0.25 μg/mL to about 0.3 μg/mL, or about 0.285 μg/mL. In embodiments, the concentration of the binding reagent for TPO in the coating solution is about 0.1 μg/mL to about 1 μg/mL, about 0.2 μg/mL to about 0.5 μg/mL, about 0.25 μg/mL to about 0.3 μg/mL, or about 0.285 μg/mL.

In embodiments, the working concentration of each detection reagent is about 0.5 μg/mL to about 20 μg/mL; about 1 μg/mL to about 10 μg/mL; or about 2 μg/mL to about 5 μg/mL. In embodiments, the working concentration of each detection reagent is about 0.5 μg/mL, about 0.6 μg/mL, about 0.7 μg/mL, about 0.8 μg/mL, about 0.9 μg/mL, about 1 μg/mL, about 1.1 μg/mL, about 1.2 μg/mL, about 1.3 μg/mL, about 1.4 μg/mL, about 1.5 μg/mL, about 1.6 μg/mL, about 1.7 μg/mL, about 1.8 μg/mL, about 1.9 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL. In embodiments, the working concentration of the detection reagent for IL-15 is about 0.1 μg/mL to about 5 μg/mL, about 0.5 μg/mL to about 3 μg/mL, or about 1 μg/mL, or about 2 μg/mL. In embodiments, the working concentration of the detection reagent for CD5 is about 0.1 μg/mL to about 5 μg/mL, about 0.5 μg/mL to about 3 μg/mL, or about 1 μg/mL, or about 2 μg/mL. In embodiments, the working concentration of the detection reagent for Flt-3L is about 0.1 μg/mL to about 5 μg/mL, about 0.5 μg/mL to about 3 μg/mL, or about 1 μg/mL, or about 2 μg/mL. In embodiments, the working concentration of the detection reagent for salivary amylase is about 5 μg/mL to about 20 μg/mL, about 8 μg/mL to about 15 μg/mL, or about 10 μg/mL. In embodiments, the working concentration of the detection reagent for CD20 is about 0.1 μg/mL to about 5 μg/mL, about 0.5 μg/mL to about 3 μg/mL, or about 1 μg/mL, or about 2 μg/mL. In embodiments, the working concentration of the detection reagent for IL-18 is about 0.1 μg/mL to about 5 μg/mL, about 0.5 μg/mL to about 3 μg/mL, or about 1 μg/mL, or about 2 μg/mL. In embodiments, the working concentration of the detection reagent for CD27 is about 0.1 μg/mL to about 5 μg/mL, about 0.5 μg/mL to about 3 μg/mL, or about 1 μg/mL, or about 2 μg/mL. In embodiments, the working concentration of the detection reagent for TPO is about 0.1 μg/mL to about 5 μg/mL, about 0.5 μg/mL to about 3 μg/mL, or about 1 μg/mL, or about 2 μg/mL.

In embodiments, the working concentration of the non-immobilized competing reagent is about 0.1 μg/mL to about 10 μg/mL; about 0.5 μg/mL to about 5 μg/mL; or about 1 μg/mL to about 3 μg/mL. In embodiments, the working concentration of the non-immobilized competing reagent for salivary amylase is about 0.5 μg/mL to about 5 μg/mL; about 1 μg/mL to about 5 μg/mL; or about 2 μg/mL.

In embodiments, the multiplexed immunoassay described herein further comprises measuring the concentration of one or more calibration reagents. In embodiments, a calibration reagent comprises a known concentration of a biomarker, e.g., IL-15, CD5, Flt-3L, salivary amylase, CD20, IL-18, CD27, or TPO. In embodiments, the calibration reagent comprises a mixture of known concentrations of multiple biomarkers, e.g., the at least four biomarkers measured in the multiplexed immunoassay. In embodiments, the multiplexed immunoassay further comprises measuring the concentration of multiple calibration reagents comprising a range of concentrations for one or more biomarkers. In embodiments, the multiple calibration reagents comprise concentrations of one or more biomarkers near the upper and lower limits of quantitation for the immunoassay. In embodiments, the multiple concentrations of the calibration reagent spans the entire dynamic range of the immunoassay. In embodiments, the calibration reagent is a negative control, i.e., containing no biomarkers.

In embodiments, the dynamic range of the assays described herein is about 0.01 pg/mL to about 5 μg/mL. In embodiments, the concentration of biomarker in the calibration reagent is about 0.01 pg/mL to about 5 μg/mL; about 0.05 pg/mL to about 4 μg/mL; about 0.1 pg/mL to about 3 μg/mL; about 0.2 pg/mL to about 1 μg/mL; about 0.3 pg/mL to about 0.5 μg/mL; about 0.4 pg/mL to about 0.1 μg/mL; about 0.5 pg/mL to about 90 ng/mL; about 0.6 pg/mL to about 80 ng/mL; about 0.7 pg/mL to about 70 ng/mL; about 0.8 pg/mL to about 60 ng/mL; about 0.9 pg/mL to about 50 ng/mL; about 1 pg/mL to about 40 ng/mL; about 2 pg/mL to about 30 ng/mL; about 3 pg/mL to about 20 ng/mL; about 4 pg/mL to about 10 ng/mL; about 5 pg/mL to about 5 ng/mL; about 6 pg/mL to about 4 ng/mL; about 7 pg/mL to about 3 ng/mL; about 8 pg/mL to about 2 ng/mL; about 9 pg/mL to about 1 ng/mL; about 10 pg/mL to about 700 pg/mL; about 20 pg/mL to about 600 pg/mL; about 30 pg/mL to about 500 pg/mL; about 40 pg/mL to about 400 pg/mL; about 50 pg/mL to about 300 pg/mL; about 60 pg/mL to about 200 pg/mL; about 70 pg/mL to about 150 pg/mL; about 80 pg/mL to about 120 pg/mL; or about 90 pg/mL to about 100 pg/mL.

In embodiments, the calibration reagent comprises about 1000 pg/mL to about 3000 pg/mL IL-15, about 3000 pg/mL to about 10000 pg/mL CD5, about 5000 pg/mL to about 10000 pg/mL Flt-3L, about 1000000 pg/mL to about 5000000 pg/mL salivary amylase, and/or about 50000 pg/mL to about 150000 pg/mL CD20. In embodiments, the calibration reagent comprises about 200 pg/mL to about 500 pg/mL IL-15, about 500 pg/mL to about 2000 pg/mL CD5, about 1000 pg/mL to about 5000 pg/mL Flt-3L, about 100000 pg/mL to about 1000000 pg/mL salivary amylase, and/or about 10000 pg/mL to about 50000 pg/mL CD20. In embodiments, the calibration reagent comprises about 50 pg/mL IL-15 to about 100 pg/mL, about 100 pg/mL to about 500 pg/mL CD5, about 100 pg/mL to about 500 pg/mL Flt-3L, about 50000 pg/mL to about 150000 pg/mL salivary amylase, and/or about 1000 pg/mL to about 5000 pg/mL CD20. In embodiments, the calibration reagent comprises about 10 pg/mL to about 20 pg/mL IL-15, about 10 pg/mL to about 100 pg/mL CD5, about 10 pg/mL to about 100 pg/mL Flt-3L, about 10000 pg/mL to about 20000 pg/mL salivary amylase, and/or about 100 pg/mL to about 1000 pg/mL CD20. In embodiments, the calibration reagent comprises about 1 pg/mL to about 10 pg/mL IL-15, about 1 pg/mL to about 20 pg/mL CD5, about 1 pg/mL to about 20 pg/mL Flt-3L, about 1000 pg/mL to about 5000 pg/mL salivary amylase, and/or about 50 pg/mL to about 200 pg/mL CD20. In embodiments, the calibration reagent comprises about 0.1 pg/mL to about 1 pg/mL IL-15, about 0.5 pg/mL to about 5 pg/mL CD5, about 1 pg/mL to about 5 pg/mL Flt-3L, about 100 pg/mL to about 1000 pg/mL salivary amylase, and/or about 10 pg/mL to about 50 pg/mL CD20. In embodiments, the calibration reagent comprises about 0.05 pg/mL to about 0.2 pg/mL IL-15, about 0.1 pg/mL to about 0.5 pg/mL CD5, about 0.1 pg/mL to about 1 pg/mL Flt-3L, about 50 to about 200 pg/mL salivary amylase, and/or about 1 pg/mL to about 10 pg/mL CD20.

In embodiments, the calibration reagent comprises about 2 ng/mL IL-15, about 6 ng/mL CD5, about 7 ng/mL Flt-3L, about 2 μg/mL salivary amylase, and/or about 75 ng/mL CD20. In embodiments, the calibration reagent comprises about 0.4 ng/mL IL-15, about 1.2 ng/mL CD5, about 1.4 ng/mL Flt-3L, about 0.4 μg/mL salivary amylase, and/or about 15 ng/mL CD20. In embodiments, the calibration reagent comprises about 80 pg/mL IL-15, about 240 pg/mL CD5, about 280 pg/mL Flt-3L, about 0.8 μg/mL salivary amylase, and/or about 3 ng/mL CD20. In embodiments, the calibration reagent comprises about 16 pg/mL IL-15, about 48 pg/mL CD5, about 56 pg/mL Flt-3L, about 160 ng/mL salivary amylase, and/or about 0.6 ng/mL CD20. In embodiments, the calibration reagent comprises about 3.2 pg/mL IL-15, about 9.6 pg/mL CD5, about 11.2 pg/mL Flt-3L, about 3.2 ng/mL salivary amylase, and/or about 120 pg/mL CD20. In embodiments, the calibration reagent comprises about 0.64 pg/mL IL-15, about 1.92 pg/mL CD5, about 2.24 pg/mL Flt-3L, about 640 pg/mL salivary amylase, and/or about 24 pg/mL CD20. In embodiments, the calibration reagent comprises about 0.128 pg/mL IL-15, about 0.384 pg/mL CD5, about 0.448 pg/mL Flt-3L, about 128 pg/mL salivary amylase, and/or about 4.8 pg/mL CD20.

In embodiments, calibration curves are generated for each biomarker for each experiment using duplicates of at least 4, 5, 6, 7, 8, 9, or 10 known levels of a mixture of calibration reagents, and the signal levels of biomarkers with unknown concentrations in test samples in that experiment are back-fitted to the calibration curve to calculate the levels of each biomarker in each test sample.

In embodiments, the multiplexed immunoassay described herein further comprises measuring the concentration of one or more biomarkers in a control reagent. In embodiments, the control reagent is obtained from an individual not exposed to radiation. In embodiments, the control reagent is obtained from an individual exposed to a known dosage of radiation. In embodiments, the measured concentration of each of the biomarkers in the control reagent is used for comparison with the measured concentrations of each of the biomarkers in the biological sample, in order to determine radiation exposure according to methods herein. In embodiments, the control reagent is a negative control reagent that comprises biomarkers at concentrations expected to correspond to negative radiation results, e.g., less than about 3 Gy, less than about 2 Gy, less than about 1 Gy, less than about 0.5 Gy, or less than about 0.1 Gy. In embodiments, the control reagent is a positive control reagent that comprises biomarkers at concentrations expected to correspond to positive radiation results, e.g., greater than about 1 Gy, greater than about 2 Gy, greater than about 3 Gy, greater than about 4 Gy, or greater than about 5 Gy.

In embodiments, concentration of biomarkers in the negative control reagent is about 1 pg/mL to about 1000 ng/mL, or about 10 pg/mL to about 500 ng/mL, or about 50 pg/mL to about 100 ng/mL, or about 100 pg/mL to about 50 ng/mL, or about 200 pg/mL to about 10 ng/mL, or about 500 pg/mL to about 1 ng/mL, depending on the biomarker. In embodiments, the concentration of biomarkers in the positive control reagent is about 10 pg/mL to about 1000 ng/mL, about 30 pg/mL to about 500 ng/mL, about 50 ng/mL to about 100 ng/mL, about 100 pg/mL to about 10 ng/mL, or about 500 pg/mL to about 1 ng/mL, depending on the particular biomarker.

In embodiments, the positive control reagent comprises about 10 pg/mL to about 20 pg/mL IL-15, about 50 pg/mL to about 200 pg/mL CD5, about 100 pg/mL to about 1000 pg/mL Flt-3L, about 50000 pg/mL to about 200000 pg/mL salivary amylase, and/or about 500 pg/mL to about 2000 pg/mL CD20. In embodiments, the positive control reagent comprises about 15 pg/mL IL-15, about 120 pg/mL CD5, about 468 pg/mL Flt-3L, about 120000 pg/mL salivary amylase, and/or about 1000 pg/mL CD20.

In embodiments, the negative control reagent comprises about 20 pg/mL to about 100 pg/mL IL-15, about 10 pg/mL to about 50 pg/mL CD5, about 1000 pg/mL to about 2000 pg/mL Flt-3L, about 200000 pg/mL to about 500000 pg/mL salivary amylase, and/or about 10 pg/mL to about 100 pg/mL CD20. In embodiments, the positive control reagent comprises about 56 pg/mL IL-15, about 35 pg/mL CD5, about 1600 pg/mL Flt-3L, about 230000 μg/mL salivary amylase, and/or about 60 pg/mL CD20.

In embodiments, the detection reagent is prepared in MSD Diluent 43 supplemented with TRITON™, mouse IgG, goat IgG, sodium chloride, and trehalose. In embodiments, the calibrator solution(s) is made in MSD Diluent 6, heated treated according to embodiments herein, and supplemented with trehelose.

In embodiments, the sample, calibration reagent, and/or control reagent is stored prior to measuring biomarker concentrations therein, e.g. using the methods described herein. In embodiments, the sample, calibration reagent, and/or control reagent is stored at about −80° C. to about 45° C., about −70° C. to about 40° C., about −60° C. to about 35° C., about −20° C. to about 30° C., about −10° C. to about 27° C., about −5° C. to about 25° C., about 0° C. to about 22° C., about 2° C. to about 15° C., about 4° C. to about 10° C. prior to being measured using a method described herein. In embodiments, the sample, calibration reagent, and/or control reagent is stored at about 2° C. to about 8° C. prior to being measured using a method described herein. In embodiments, the sample, calibration reagent, and/or control reagent is stored at about 22° C. to about 27° C. prior to being measured using a method described herein. In embodiments, the sample, calibration reagent, and/or control reagent is stored at about −70° C. prior to being measured using a method described herein. In embodiments, the sample, calibration reagent, and/or control reagent is stored for about 0 hours to about 72 hours, about 1 hour to about 60 hours, or about 2 hours to about 48 hours, or about 4 hours to about 24 hours, or about 8 hours to about 16 hours prior to being measured using a method described herein. In embodiments, the sample, calibration reagent, and/or control reagent is stored for about 0 hours to about 60 hours at about 4° C. prior to being measured using a method described herein. In embodiments, the sample, calibration reagent, and/or control reagent is stored for about 0 hours to about 48 hours at about 23° C. prior to being measured using a method described herein. In embodiments, storage of the sample as described herein (e.g., for about 48 hours at about 23° C.) does not substantially vary its biomarker concentrations. In embodiments, storage of the sample does not vary the measured biomarker concentrations by more than 5%, 10%, 20%, 25%, or 30% as compared to a sample that was measured without storage. In embodiments, the sample is a plasma sample. In embodiments, biomarker concentrations do not substantially vary upon storage of the calibration reagent as described herein (e.g., for about 24 hours at about 25° C.). In embodiments, biomarker concentrations do not substantially vary upon storage of the control reagent as described herein (e.g., for about 24 hours at about 25° C.). Methods and conditions for storing the samples, calibration reagents, and/or control reagents described herein are known to one of ordinary skill in the art.

In embodiments, biomarkers levels, e.g., IL-15, CD5, Flt-3L, salivary amylase, and CD20, in a plasma sample are not substantially affected by the presence of compounds that are commonly present in sample matrices and can interfere with biomarker measurement, also referred to herein as interferents. In embodiments, the levels of IL-15, CD5, Flt-3L, salivary amylase, and CD20 does not vary by more than 5%, 10%, 20%, 25%, or 30% in the presence or absence of an interferent. Non-limiting examples of interferents include host antibodies, such as host anti-mouse antibodies and rheumatoid factor; host plasma components, such as conjugated bilirubin, unconjugated bilirubin, hemoglobin (hemolysate), triglyceride-rich lipoproteins, pancreatic amylase, albumin, and γ globulin; plasma additives, such as EDTA; pain medications, such as ibuprofen, acetaminophen, salicylic acid, acetylsalicylic acid, and naproxen; radiation countermeasure drugs, such as Neupogen (recG-CSF), Neulasta (PEG-recG-CSF), DTPA, and potassium iodide; antibiotics, such as cefoxitin, doxycycline, ampicillin, and rifampin; anti-emetics, such as ondansetron HCl; anti-diarrheal, such as loperamide HCl; cholesterol medications, such as atorvastatin (Lipitor); diabetes medications, such as metformin (Glucophage), and allergy medications, such as loratadine (Claritin).

Ultra-High Throughput Methods

In embodiments, the disclosure further provides an automated version of the methods of the invention using an ultra high-throughput robotic liquid handling system. This system allows simultaneous preparation of up to 1,520 samples with accuracy and reproducibility unmatched by a human operator. In embodiments, the automated system is a free-standing, fully integrated system for carrying out immunoassays using ECL technology. This system, capable of simultaneously running up to twenty 96-well assay plates, includes a robotic lab automation workstation for liquid handling and plate manipulation, physically integrated with an ECL reader. In embodiments, the workflow conducts the methods described herein, e.g., the multiplexed immunoassays, with minimal human intervention. In embodiments, the ultra-high throughput system produces results for about 1,520 samples in about 30 minutes to about 300 minutes, or about 60 minutes to about 150 minutes, or about 70 minutes to about 130 minutes. The ultra-high throughput system described herein is capable of processing about 10,000 single samples in a day, or about 5,000 duplicate samples in a day.

Radiation Exposure Determination

In embodiments, the disclosure further provides a method of determining radiation exposure in a human, comprising a) conducting the multiplexed immunoassay as described herein on a biological sample of a human, b) detecting the concentration of biomarker IL-15, biomarker CD5, biomarker Flt-3L, and biomarker salivary amylase, c) determining if: (i) the concentration of biomarker IL-15 is higher compared to a control; (ii) the concentration of biomarker CD5 is lower compared to a control; (iii) if the concentration of biomarker Flt-3L is higher compared to a control; (iv) if the concentration of salivary amylase is higher or the same compared to a control, wherein if any of (i), (ii), (iii) or (iv) is true, reporting that the human has been exposed to radiation, wherein the control of (i), (ii), (iii), and (iv) is from a human who has not been exposed to radiation. The roles of IL-15, CD5, Flt-3L, and salivary amylase in radiation response are described herein. In embodiments, the determining is performed by an immunoassay, e.g., a multiplexed immunoassay described herein.

In embodiments, the detecting step of the method further comprises detecting the concentration of biomarker CD20, biomarker IL-18, biomarker CD27, biomarker TPO, or combination thereof, and wherein: (v) if the concentration of biomarker CD20 is lower compared to a control; (vi) if the concentration of biomarker IL-18 is higher compared to a control; (vii) if the concentration of biomarker CD27 is lower compared to a control; (viii) if the concentration of biomarker thyroid peroxidase (TPO) is higher compared to a control; wherein if any of (i) to (viii) is true, reporting that the human has been exposed to radiation, wherein the control of (i) to (viii) is from a human who has not been exposed to radiation. The roles of CD20, IL-18, CD27, and TPO in radiation response are described herein.

In further embodiments, the disclosure provides a method of determining radiation exposure in a human, comprising a) detecting CD5 in a biological sample of a human, b) determining if a concentration of CD5 in the biological sample is lower than a control concentration of CD5 in a non-irradiated control sample, c) if the concentration in the biological sample is lower than in the non-irradiated control sample, reporting that the human was exposed to radiation. In embodiments, the biological sample is whole blood, serum, plasma, cerebrospinal fluid, urine, saliva, or an extraction or purification therefrom, or dilution thereof. Biological samples are further described herein. As described herein, changes in CD5 levels in serum and/or plasma were discovered to be a reliable and accurate indicator of radiation exposure. In embodiments, the biological sample is serum or plasma. In embodiments, the method further comprises measuring one or more additional biomarkers selected from the group consisting of salivary amylase, IL-15, IL-18, Flt-3L and CD20. In embodiments, the concentration of CD5, salivary amylase, IL-15, IL-18, Flt-3L, and/or CD20 is determined by an immunoassay described herein, e.g., a multiplexed immunoassay.

Methods of determining radiation dose based on the measured concentrations of a combination of biomarkers are described, e.g., in U.S. Pat. No. 10,436,784 and US 2018/0246100. In embodiments, the invention provides a cost function algorithm for determining radiation dose based on the measured concentrations of a combination of biomarkers. In embodiments, the invention provides a linear regression algorithm (also referred to herein as a “linear model”) for determining radiation dose based on the measured concentrations of a combination of biomarkers. As compared with a cost function algorithm, a linear regression algorithm is more easily implemented, e.g., integrated in software. Methods of measuring the concentrations of biomarkers are described herein. In embodiments, the disclosure provides a radiation dose-calculation algorithm comprising (a) determining measured levels of a combination of radiation biomarkers in a patient sample; (b) applying the measured levels to a model that approximates a linear relationship between predicted radiation doses and predicted levels of the combination of radiation biomarkers; (c) performing a regression analysis on the model; (d) determining a calculated radiation dose based on the regression analysis; and optionally, (e) comparing the calculated radiation dose to a threshold value to classify individuals according to a dose received, for example, to distinguish exposed from non-exposed individuals or to identify patients who would benefit from a treatment option.

All or one or more parts of the algorithm(s), statistical method(s), and statistical model(s) disclosed herein can be performed by or executed on a processor, general purpose or special purpose or other such machines, integrated circuits or by any combination thereof. Moreover, the software instructions for performing the algorithm(s), statistical method(s), and statistical model(s) disclosed herein may also be stored in whole or in part on a computer-readable medium, e.g., a storage device for use by a computer, processor, general or special purpose or other such machines, integrated circuits or by any combination thereof. A non-limiting list of suitable storage devices includes but is not limited to a computer hard drive, compact disk, transitory propagating signals, a network, and/or a portable media device to be read by an appropriate drive or via an appropriate connection.

Various statistical models and algorithms can be utilized to calculate the radiation exposure based on levels of biomarkers present in a sample, e.g., sample of a patient of a particular species. In an embodiment, a cost function algorithm can be utilized to calculate a quality of match for various known levels of biomarkers at various radiation doses. The cost function algorithm can utilize a cost function model given by the equation:

$$F ( dose ) = {\sum }_{i = 1}^{n} log ( \frac{{m}_{i} + {LOD}_{i}}{{M}_{i} ( dose ) + {LOD}_{i}} )$$

where mᵢ is a measured level of biomarker i of n number of biomarkers, LOD is an assay detection limit, and Mᵢ(dose) is a concentration on a response surface for a given dose of radiation (e.g., predicted biomarker level as a function of dose at a known time post-exposure). Median values for any given biomarker can be plotted versus dose and time to create a predicted response surface. A complete description of the algorithm utilizing the cost function model can be found in U.S. Patent Application Pub. No. 2018/0246100, the entire contents of which are incorporated herein by reference.

In an embodiment, a linear model can be trained and analyzed with a linear regression algorithm (“linear model algorithm”) to calculate the radiation exposure based on level of biomarkers present. As described herein, a linear regression algorithm includes simpler calculations and is therefore more easily implemented in software. The linear model can be given in the following generalized forms depending on whether the biomarker concentrations are utilized as-is or after log transformation:

$$Dose = {A}_{0} + {A}_{1} T + {\sum }_{i = 2}^{n + 2} {{A}_{i} [ Biomarker ]}_{i} or$$

$$Dose = {A}_{0} + {A}_{1} T + {\sum }_{i = 2}^{n + 2} {A}_{i} {{log}_{10} [ Biomarker ]}_{i}$$

Based on the assumption that the changes in biomarker concentration (in linear or log-transformed space) with radiation will be similar in different species, although the normal biomarker levels may be different, use of concentrations that are normalized to the median normal concentration may allow for use of a single equation a set of coefficients for multiple species as in:

$$Dose = {A}_{0} + {A}_{1} T + {\sum }_{i = 2}^{n + 2} {A}_{i} ( {[ Biomarker ]}_{i} - {[ Biomarker ]}_{i , n o r m} ) or$$

$$Dose = {A}_{0} + {A}_{1} T + {\sum }_{i = 2}^{n + 2} {A}_{i} {log}_{10} \frac{{[ Biomarker ]}_{i}}{{[ Biomarker ]}_{i , n o r m}}$$

In some cases, it may be beneficial to add non-linear terms, which have non-linear combinations of one or more of the inputs (time or biomarker concentrations). In particular, since AMY tends to initially increase and then decrease with time over the period of interest for dose estimation, the relationship of dose with [AMY] be better represented if the AMY concentration or log-transformed AMY concentration is replaced in the equation by the concentration or log-transformed concentration divided by time.

In the different forms of the linear model shown above, T is a known exposure time to a radiation dose, [Biomarker]ᵢ is a measured level of a biomarker for i=1 to n biomarkers, [Biomarker]_{i,norm} is a median normal level of the ith biomarker for a species being tested, and Aₚ is a (p+1) parameter vector (or regression coefficients) where A₀ is an intercept term.

Typically when using linear models, a training data set will be used to fit the model by linear regression and determine the coefficients for the model. A variety of methodologies are known in the art for fitting such models. In one embodiment, a training methodology is used that is designed to prevent over-fitting, such as Ridge, Lasso and Elastic Net regression approaches. In an embodiment, a ridge regression can be utilized to avoid overfitting. The ridge regression fitting can include a penalty, scaled by a factor, λ, for large coefficients, where a 5-fold x-validation analysis is utilized to select an optimal value of λ.

In an example, the cost function algorithm and the linear model algorithm where tested and analyzed for the particular set of [biomarkers] ([AMY], [CD5], [CD20] [Flt3L], [IL15]). In this example, the linear model can be given by the equation:

$$Dose = {A}_{0} + {A}_{1} T + \frac{{A}_{2}}{T} {log}_{10} \frac{[ AMY ]}{{[ AMY ]}_{n o r m}} + {A}_{3} {log}_{10} \frac{[ C D 5 ]}{{[ C D 5 ]}_{n o r m}} + {A}_{4} {log}_{10} \frac{[ C D 20 ]}{{[ C D 20 ]}_{n o r m}} + {A}_{5} {log}_{10} \frac{[ F l t 3 L ]}{{[ F l t 3 L ]}_{n o r m}} + {A}_{6} {log}_{10} \frac{[ IL 15 ]}{{[ IL 15 ]}_{n o r m}}$$

where T is a known exposure time to a radiation dose, [Biomarker] (e.g., [AMY]) is a measured level of a biomarker, [Biomarker]ₙₒᵣₘ (e.g., [AMY]ₙₒᵣₘ) is a median normal level of a biomarker for a species being tested, and A_{1 . . . 6} is parameter vector (or regression coefficients) where A₀ is an intercept term. In this equation, the normalized and transformed [AMY] value is divided by T to account for a drop in biomarker level over time. To determine the dosage, a linear regression can be utilized to fit the above equation. In an embodiment, a ridge regression can be utilized to avoid overfitting. The ridge regression fitting can include a penalty, scaled by a factor, λ, for large coefficients, where a 5-fold x-validation analysis is utilized to select an optimal value of λ.

In this example, the cost function model algorithm and the linear model algorithm can be trained with NHP dose response studies and model response surfaces for each [biomarker] using spline fit to interpolate between points. When utilizing the cost function trained with NHP dose response surfaces, the response surfaces can be adjusted when utilized for predicting radiation dose for human patients. Each base line from the NHP dose response surfaces can be normalized to match median human baseline levels. For radiation response, the magnitude of response (e.g., fold changes) can be maintained. For dose scaling, multiplicative scaling can be applied, for example, Dₕᵤₘₐₙ=0.6666*D_{NNP} (e.g., 2 Gy for human=3 Gy for rhesus).

When utilizing the linear model algorithm trained with NHP dose response surfaces, the response surfaces can be adjusted when utilized for predicting radiation dose for human patients. A baseline correction may not be necessary as the linear model equation normalizes for species differences in the baseline values. For dose scaling, A values for human patient can be scaled by two thirds for NHP dose response surfaces, ⅔*A (e.g., 3 Gy for NHP=2 Gy for human patients).

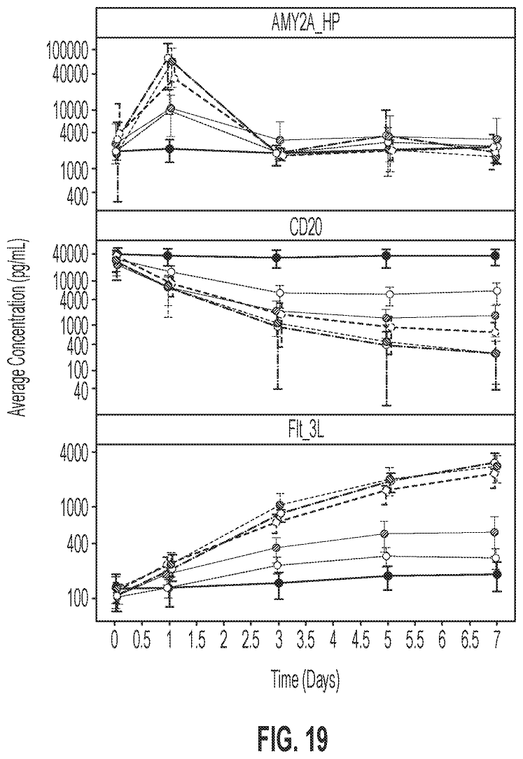

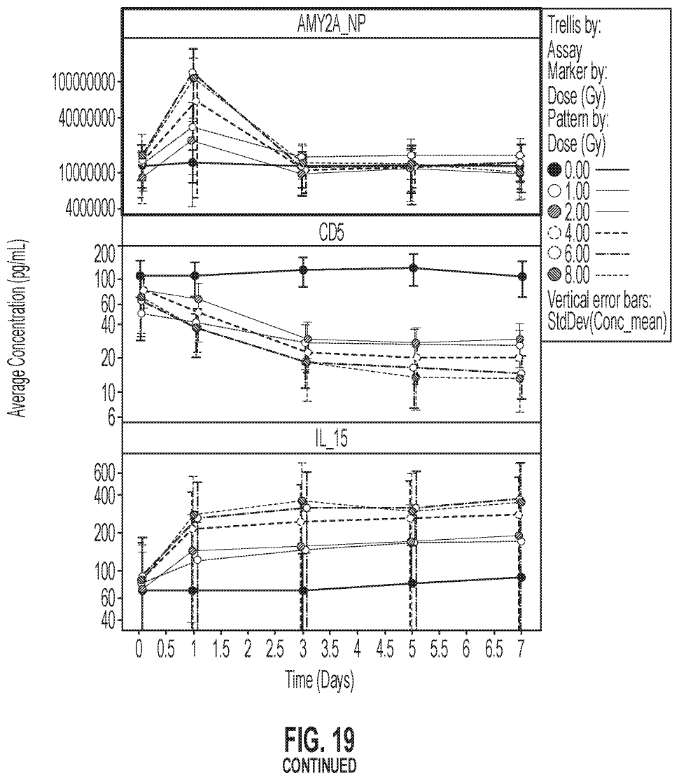

FIG. 19 illustrates an example NHP dose response data set that can be utilized to train the cost function algorithm and the linear model algorithm. As illustrated, the results for [CD5], [CD20] [Flt3L], [IL15] from a human panel and [AMY] from the NHP specific assay were utilized to train the cost function algorithms and the linear algorithms. The study exposed animals to six doses of radiation including a zero (0) Gy sham condition. Samples were collected at five (5) time points including a zero (0) day point prior to exposure. Each point represents an average biomarker level measured across samples from ten (10) animals. Results from the five (5) biomarker assays in the biodosimetry panels without bold-lined borders. Because the [AMY] assay is optimized for human samples and amylase [AMY1A] and does not quantitate all samples from normal NHP, the samples were also tested with a separate amylase assay that was optimized for measuring amylase in NHP samples [AMY2A] (panel with bold-lined borders).

In this example, random sub-sampling was utilized to characterize the robustness of the cost function algorithm and the linear model algorithm. The random sub-sampling was also utilized to select optimal cutoff values for classifying samples as above or below a critical triage dose (e.g., 3 Gy for NHP). In the random sub-sampling, eight (8) out of ten (10) animals were randomly selected for each of the six (6) dose conditions. The random sub-sampling was then utilized to train the cost function algorithm and the linear model algorithm. For the cost function algorithm, the random sub-sampling was used as a training set to create dose response surfaces. For the linear model algorithm, the random sub-sampling was used as a training set to fit the linear regression equation. The two (2) remaining animal form the set were utilized to evaluate the performance per dose condition. A Receiver Operating Characteristic (ROC) analysis was utilized to measure sensitivity and specificity for distinguishing doses above and below three (3) Gy as a function of the cutoff predicted dose used for classification. This process was repeated two hundred and fifty (250) times and the sensitivity and the specificity were plotted with median and approximately 95% confidence intervals as a function of cut-off. From the plots, the optimal cutoff value was selected. In all the analyses, samples form non-irradiated subjects (e.g., dose=0 Gy) were included four (4) times in the analysis with four different assigned time values (e.g., one (1), three (3), five (5), and seven (7) days).

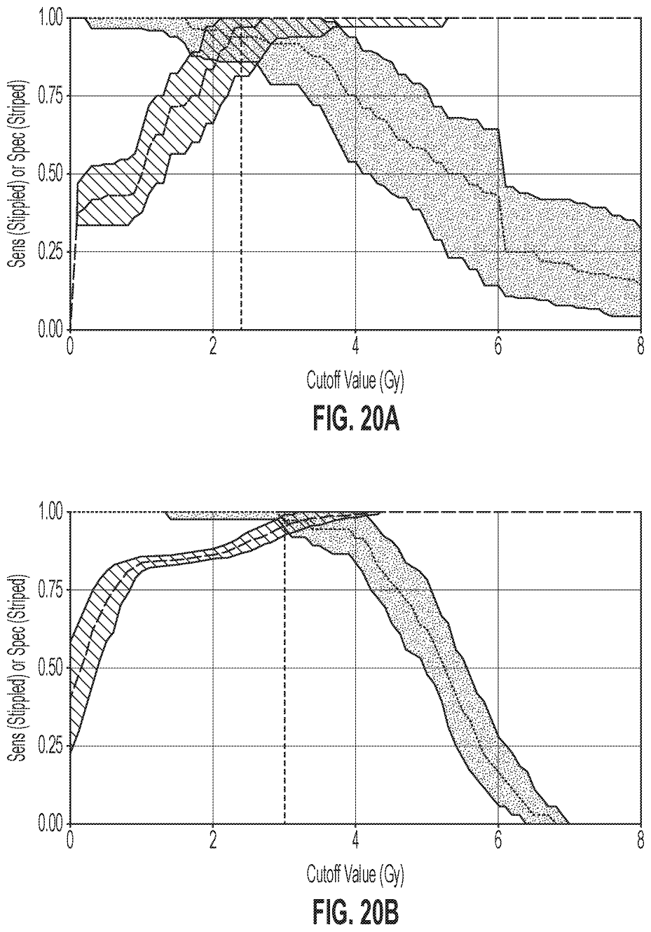

FIGS. 20A and 20B illustrate an example of the sensitivity and specificity plot for the cost function algorithm and the linear model algorithm. As illustrated, measured specificity (striped) and sensitivity (stippled) for the random sub-sampling were plotted as a function of the cutoff value and used to classify samples as above or below critical triage dose (e.g., 3 Gy for NHP). FIGS. 20A and 20B show median values (lines inside the striped or stippled regions) and approximately 95% confidence intervals (striped or stippled regions) for each characteristic for each of the cost function algorithm and the linear model algorithm. The optimal cutoff for the cost function algorithm was a predicted dose of around 2.4 Gy for NHP, which corresponds to 1.6 Gy for humans. The optimal cutoff for the linear model algorithm was a predicted dose of around 3.0 Gy for NHP, which corresponds to 2.0 Gy for humans.

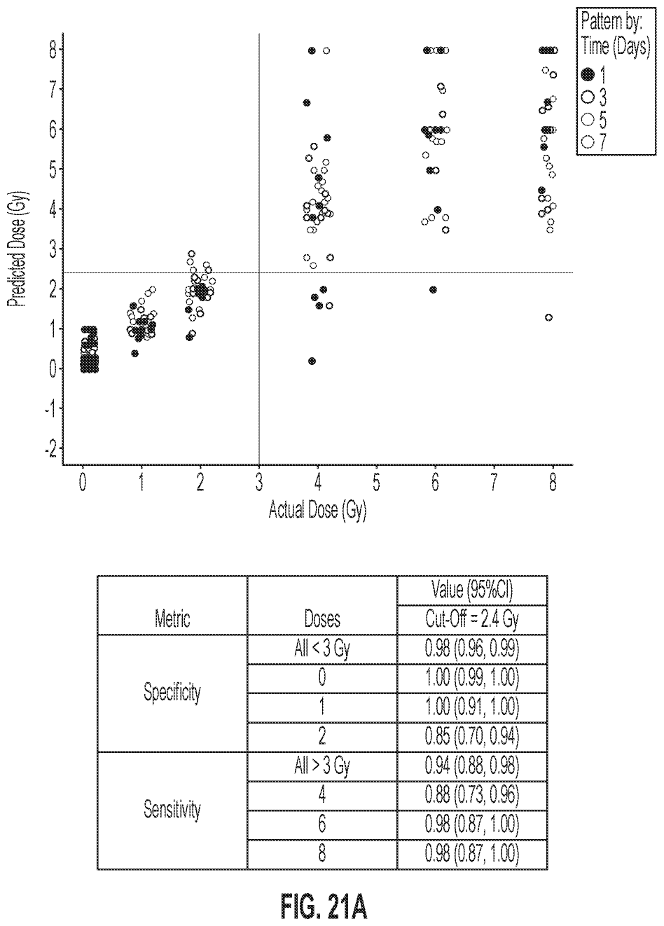

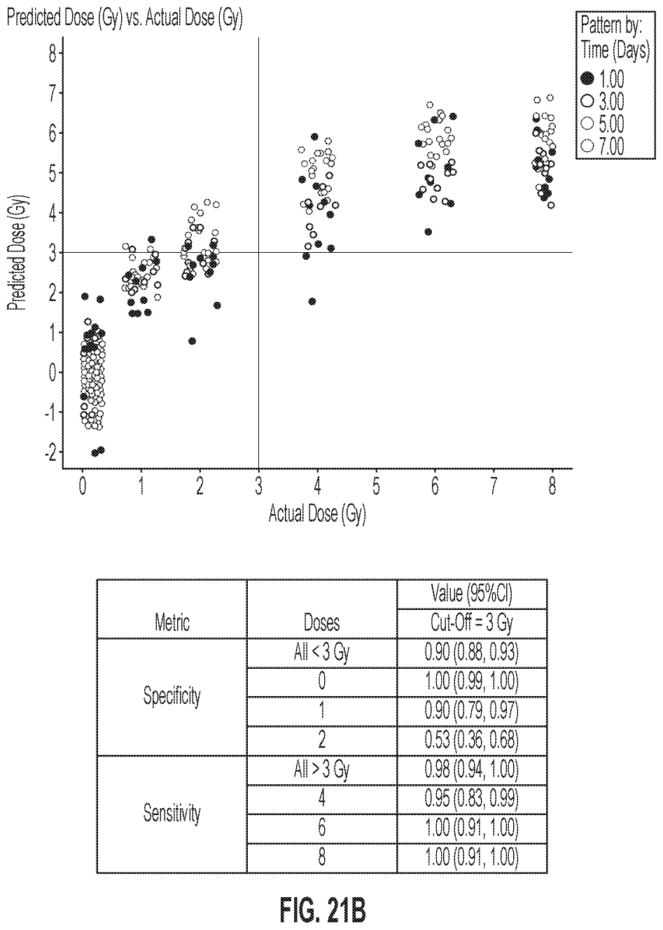

Using the NHP, the accuracy of the cost function algorithm and the linear model algorithm were evaluated using a complete data set from NHP dose response study. FIGS. 21A and 21B illustrate an example of the accuracy of the cost function algorithm and the linear model algorithm. FIGS. 21A and 21B show predicted dose as a function of actual dose with points indicated with different outline patterns based on time from exposure. The dashed vertical line is the critical triage threshold, and the dashed horizontal line is the optimal cutoff value for classifying samples as above or below the triage threshold. The Tables in FIGS. 21A and 21B provide the classification accuracy for all negative and positive samples, or stratified by dose. The approximately 95% confidence intervals were estimated based on the binomial distribution. Column 3 of Tables in FIG. 21A (cost function algorithm) and FIG. 21B (linear model algorithm) show the approximately 95% confidence interval range based on dose. As seen from FIGS. 21A and 21B, the cost function algorithm and the linear model algorithm provided good classification accuracy. The cost function algorithm provided better specificity at the high negative dose (e.g., 2 Gy). The linear model algorithm provided better sensitivity at the low positive dose (e.g., 4 Gy). In embodiments, the 95% confidence interval for specificity in determining doses of less than 3 Gy, is about 0.35 to about 1, about 0.4 to about 1, about 0.45 to about 1, about 0.5 to about 1, about 0.55 to about 1, about 0.6 to about 1, about 0.65 to about 1, about 0.7 to about 1, about 0.75 to about 1, about 0.8 to about 1, about 0.85 to about 1, about 0.9 to about 1, about 0.91 to about 1, about 0.92 to about 1, about 0.93 to about 1, about 0.94 to about 1, about 0.95 to about 1, about 0.96 to about 1, about 0.97 to about 1, about 0.98 to about 1, about 0.99 to about 1, or about 0.5, about 0.53, about 0.55, about 0.57, about 0.6, about 0.63, about 0.65, about 0.67, about 0.7, about 0.73, about 0.75, about 0.77, about 0.8, about 0.83, about 0.85, about 0.87, about 0.9, about 0.91, about 0.92, about 0.93, about 0.94, about 0.95, about 0.96, about 0.97, about 0.98, about 0.99, or about 1. In embodiments, the 95% confidence interval for sensitivity in determining doses of greater than 3 Gy is about 0.7 to about 1, about 0.73 to about 1, about 0.75 to about 1, about 0.77 to about 1, about 0.8 to about 1, about 0.83 to about 1, about 0.85 to about 1, about 0.87 to about 1, about 0.9 to about 1, about 0.91 to about 1, about 0.92 to about 1, about 0.93 to about 1, about 0.94 to about 1, about 0.95 to about 1, about 0.96 to about 1, about 0.97 to about 1, about 0.98 to about 1, about 0.99 to about 1, about 0.7, about 0.73, about 0.75, about 0.77, about 0.8, about 0.83, about 0.85, about 0.87, about 0.9, about 0.91, about 0.92, about 0.93, about 0.94, about 0.95, about 0.96, about 0.97, about 0.98, about 0.99, or about 1.

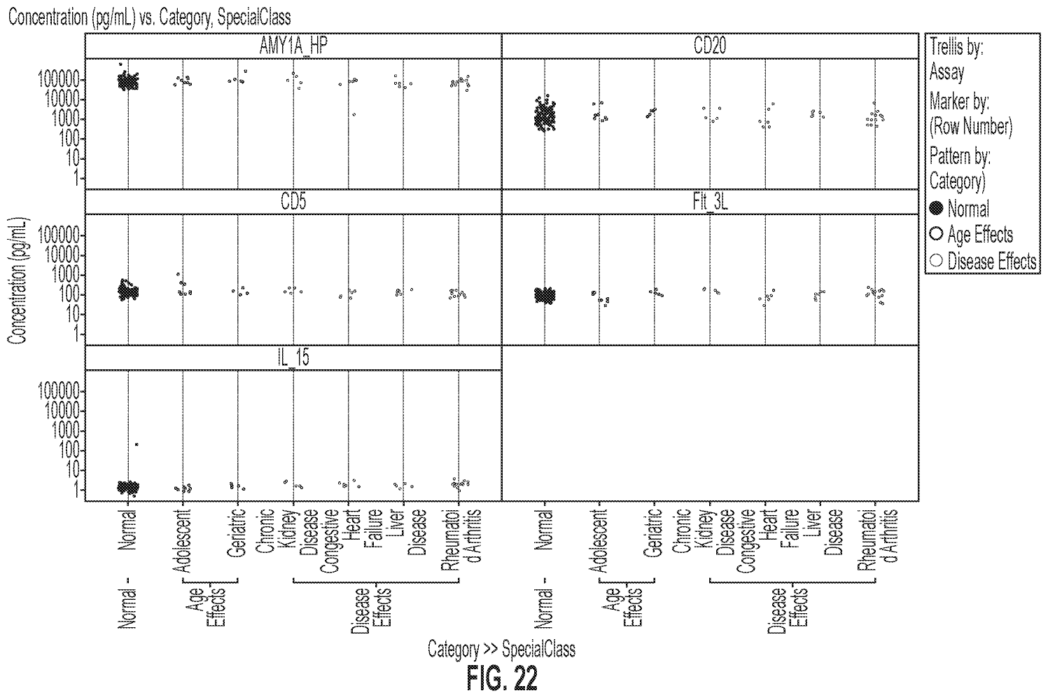

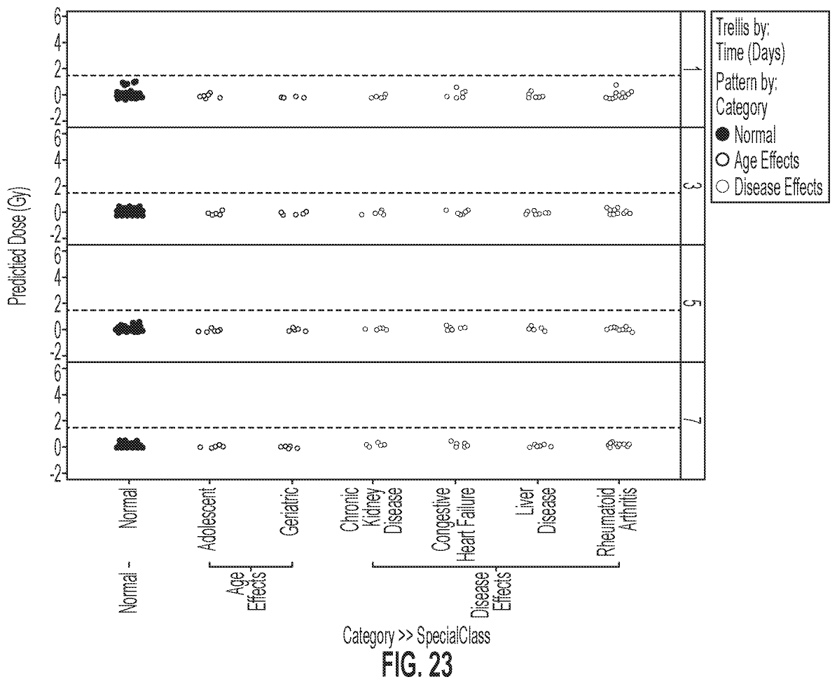

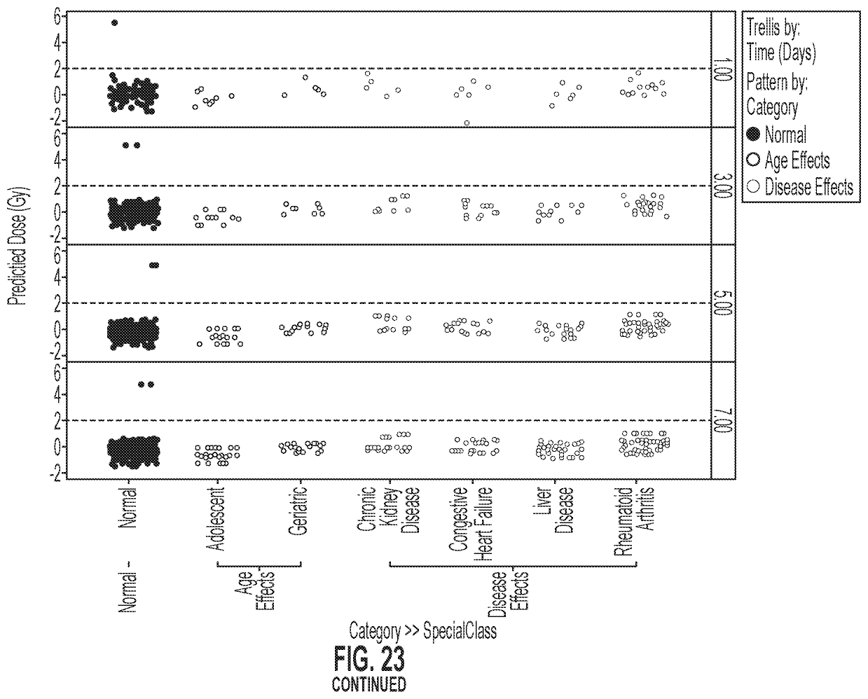

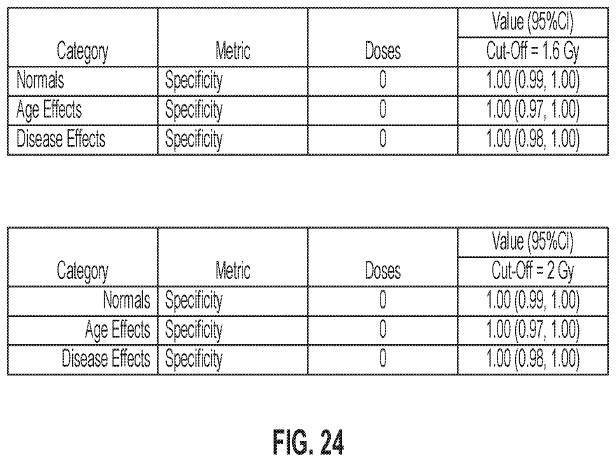

In another example, measured biomarker values from human patients for the particular set of [biomarkers] ([AMY], [CD5], [CD20] [Flt3L], [IL15]) were used to test the specificity of the cost function algorithm and the linear model algorithm. FIG. 22 illustrates the data from human patients used to test the cost function algorithm and the linear model algorithm. As illustrated, biomarker levels in plasma from one hundred and thirty-six (136) normal adult human donors were used. Biomarker levels in plasma from between five (5) to fourteen (14) samples from six (6) special populations (adolescents, geriatrics, chronic kidney disease, congestive heart failure, liver disease and rheumatoid arthritis) were also utilized. The data was applied to the cost function algorithm and the linear model algorithm to evaluate specificity using classification cutoff for predicted dose of 1.6 Gy for cost function and 2.0 Gy for linear model algorithm. FIG. 23 illustrates an example of the results for the test of the cost function algorithm and the linear model algorithm using the data from FIG. 22. FIG. 23 shows predicted doses for normal humans and special human populations (by age or disease) using the cost function algorithm. FIG. 23 shows predicted doses for normal humans and special human populations (by age or disease) using the linear model algorithm. The Tables in FIG. 24 show the observed specificities for the different classes of subjects. The approximately 95% confidence intervals were estimated based on a binomial distribution. Column 4 of Tables in FIG. 24 top panel (cost function algorithm) and FIG. 24 bottom panel (linear model algorithm) show the approximately 95% confidence interval range for specificity based on dose. As shown, the cost function algorithm and the linear model algorithm both demonstrate excellent specificity. In embodiments, the 95% confidence interval for specificity in determining a radiation dose based on a cut-off value of about 1.5, about 1.6, about 1.7, about 1.8, about 1.9, or about 2 Gy is about 0.8 to about 1, about 0.85 to about 1, about 0.9 to about 1, about 0.91 to about 1, about 0.92 to about 1, about 0.93 to about 1, about 0.94 to about 1, about 0.95 to about 1, about 0.96 to about 1, about 0.97 to about 1, about 0.98 to about 1, about 0.99 to about 1, or about 0.8, about 0.83, about 0.85, about 0.87, about 0.9, about 0.91, about 0.92, about 0.93, about 0.94, about 0.95, about 0.96, about 0.97, about 0.98, about 0.99, or about 1.

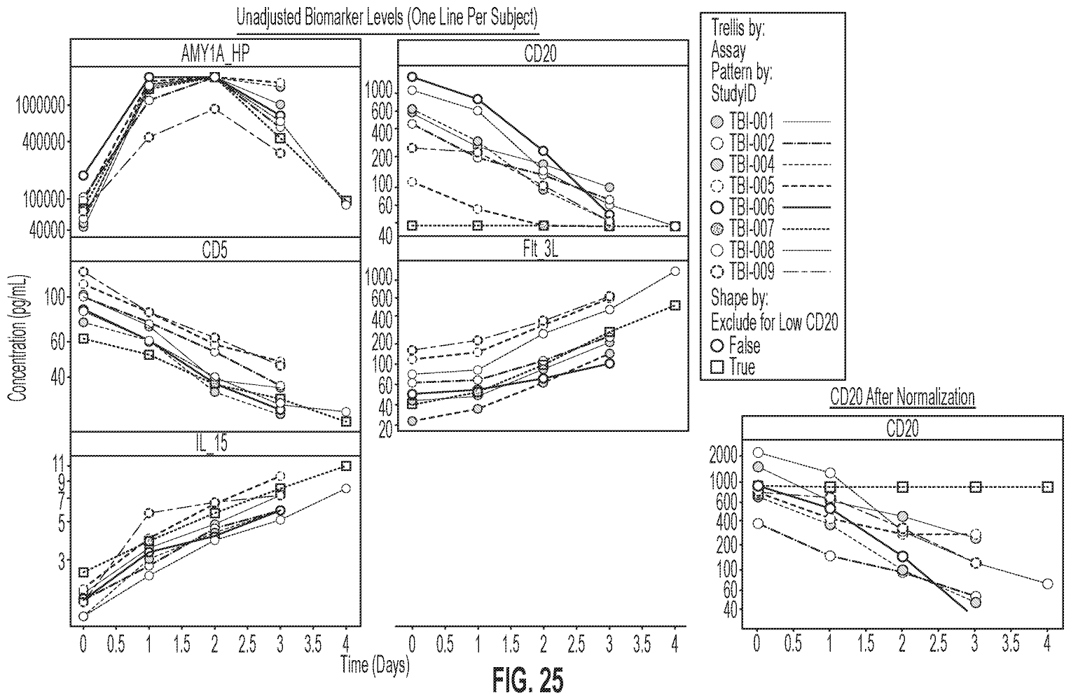

In another example, clinical trial data from a human stem cell transplant (SCT) data set was used to evaluate performance of the cost function algorithm and the linear model algorithm the particular set of [biomarkers] ([AMY], [CD5], [CD20] [Flt3L], [IL15]). FIG. 25 illustrates the data from the SCT data set. As illustrated, changes in radiation biomarker levels in human cancer patients exposed to radiation prior to stem cell transplant were plotted. In the clinical trial, samples were tested from nine (9) patients receiving 13.75 Gy in 1.25 Gy fractions over four (4) days. Samples were collected prior to exposure, e.g., day zero (0), and on days one (1) to four (4). In the present study, patients with baseline CD20<60 pg/mL are indicated with open circles. Per a clinical study plan, atypically low [CD20] in the patients were addressed by excluding any patients with [CD20] at or below the detection limit of the assay, e.g., <60 pg/mL. FIG. 25 shows original CD20 values. FIG. 25 after normalization to correct for atypically low baseline levels in the SCT patient population.

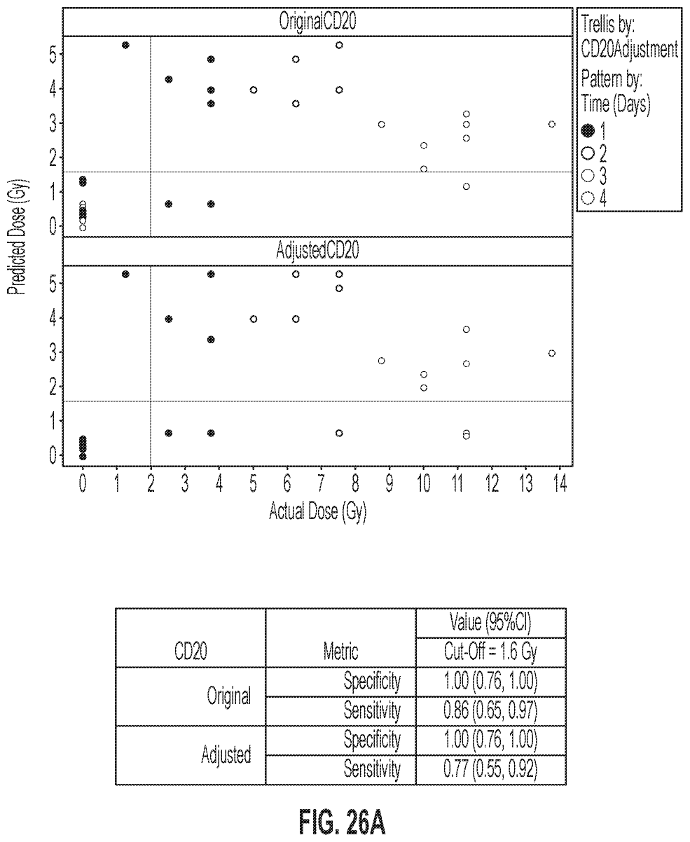

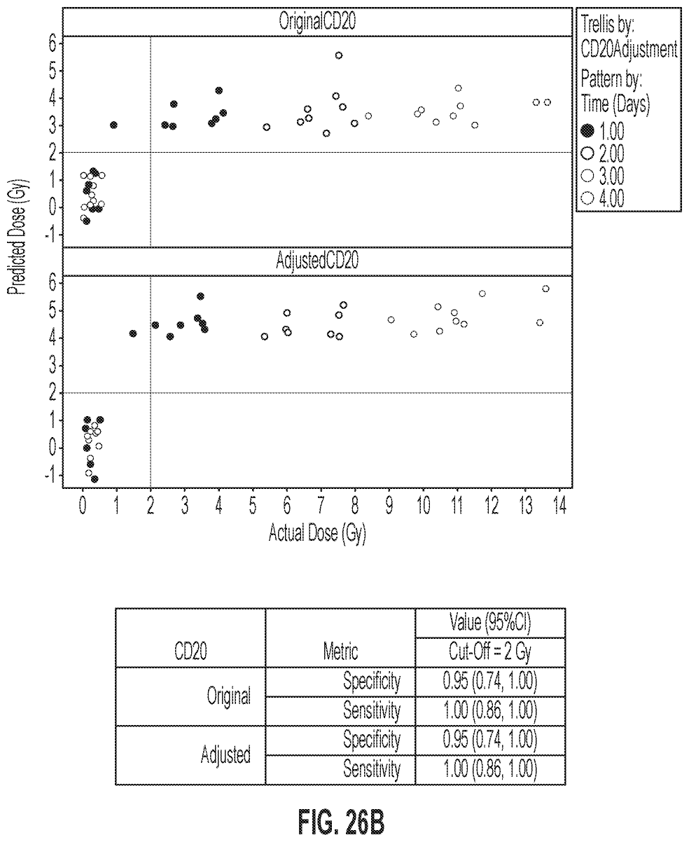

In this example, the accuracy of the cost function algorithm was analyzed using classification cutoff for predicted dose of 1.6 Gy. The accuracy of the linear model algorithm was analyzed using classification cut off for a predicted does of 2.0 Gy. The performance was evaluated using original [CD20] values and normalized CD20 values, FIG. 25. FIGS. 26A and 26B illustrate the results of the evaluation. FIG. 26A shows dose prediction for SCT patient samples as a function of total dose (accumulated in 1.25 G fractions) for the cost function algorithm. Column 3 of the Table in FIG. 26A shows the approximately 95% confidence interval range based on dose. FIG. 26B shows dose prediction for SCT patient samples as a function of total dose (accumulated in 1.25 G fractions) for the linear model algorithm. Column 3 of the Table in FIG. 26B shows the approximately 95% confidence interval range based on dose. In FIGS. 26A and 26B, points are indicated with different outline patterns based on time from first fraction. For both figures, there are two subplots: the top used the original [CD20] values for dose prediction and the bottom uses [CD20] values that were normalized to account for the atypically low [CD20] baseline value in SCT patients. The [CD20] adjustment unexpectedly had minimal effects on the performance of either algorithm. Overall, the linear model algorithm likely performs better because the algorithm was more tolerant of outlying individual biomarkers. In embodiments, the 95% confidence interval for specificity in determining radiation dose using a cut-off value of about 1.5, about 1.6, about 1.7, about 1.8, about 1.9, or about 2 Gy is about 0.5 to about 1, about 0.55 to about 1, about 0.6 to about 1, about 0.65 to about 1, about 0.7 to about 1, about 0.75 to about 1, about 0.8 to about 1, about 0.85 to about 1, about 0.9 to about 1, about 0.91 to about 1, about 0.92 to about 1, about 0.93 to about 1, about 0.94 to about 1, about 0.95 to about 1, about 0.96 to about 1, about 0.97 to about 1, about 0.98 to about 1, about 0.99 to about 1, or about 0.5, about 0.53, about 0.55, about 0.57, about 0.6, about 0.63, about 0.65, about 0.67, about 0.7, about 0.73, about 0.75, about 0.77, about 0.8, about 0.83, about 0.85, about 0.87, about 0.9, about 0.91, about 0.92, about 0.93, about 0.94, about 0.95, about 0.96, about 0.97, about 0.98, about 0.99, or about 1. In embodiments, the 95% confidence interval for sensitivity in determining radiation dose using a cut-off value of about 1.5, about 1.6, about 1.7, about 1.8, about 1.9, or about 2 Gy is about 0.5 to about 1, about 0.55 to about 1, about 0.6 to about 1, about 0.65 to about 1, about 0.7 to about 1, about 0.75 to about 1, about 0.8 to about 1, about 0.85 to about 1, about 0.9 to about 1, about 0.91 to about 1, about 0.92 to about 1, about 0.93 to about 1, about 0.94 to about 1, about 0.95 to about 1, about 0.96 to about 1, about 0.97 to about 1, about 0.98 to about 1, about 0.99 to about 1, or about 0.5, about 0.53, about 0.55, about 0.57, about 0.6, about 0.63, about 0.65, about 0.67, about 0.7, about 0.73, about 0.75, about 0.77, about 0.8, about 0.83, about 0.85, about 0.87, about 0.9, about 0.91, about 0.92, about 0.93, about 0.94, about 0.95, about 0.96, about 0.97, about 0.98, about 0.99, or about 1.

In embodiments, the disclosure provides an injury severity calculation algorithm that comprises (a) determining measured levels of a combination of radiation biomarkers in a patient sample; (b) applying the measured levels to a model that approximates a linear relationship between predicted radiation doses and predicted levels of the combination of radiation biomarkers; (c) performing a regression analysis on the model; (d) determining a calculated radiation dose based on the regression analysis; and optionally, (e) comparing the calculated radiation dose to a threshold value to classify individuals according to a dose received, for example, to distinguish exposed from non-exposed individuals or to identify patients who would benefit from a treatment option.

The concentrations of biomarkers measured in the biological samples described herein, particularly in the biological samples obtained from individuals exposed to a known dose of radiation, can be used to train and characterize the algorithms described herein. In embodiments, random sub-sampling of the measured concentrations is used to avoid training bias. For example, the data can be randomly split (e.g., in a 4:2 ratio) into a “training set” and a “test set,” and the performance of the algorithm is measured with the test set. The process of randomly splitting the data for training and testing can be repeated many times to improve the trained algorithm.

Prediction models generated from the algorithms provided herein for determining radiation exposure can be trained using measured biomarker concentrations from samples obtained from one or more subjects exposed to a known amount of radiation, optionally samples obtained at different time intervals after radiation exposure. In embodiments, the samples for training the models are obtained from a non-human subject. In embodiments, the samples for training the models are obtained from a nonhuman primate, a mouse, or a rat. Non-limiting examples of nonhuman primates include monkey, including, e.g., rhesus monkey, squirrel monkey, pig-tailed monkey, baboon, macaque, marmoset, mangabey, lemur, and capuchin. Models trained using non-human samples can be used to assess radiation exposure in humans by accounting for the differential radiation sensitivity between species. For example, humans have approximately a 2.5-fold higher sensitivity to radiation dose relative to a mouse model, and thus a measured radiation exposure of approximately 9-10 Gy for a mouse model would be approximately 3-4 Gy in a human. In another example, humans have approximately a 1.4-fold higher sensitivity to radiation dose relative to a nonhuman primate (NHP) model, and thus a measured radiation exposure of approximately 4-7 Gy for an NHP model would be approximately 3-5 Gy in a human. The estimated critical threshold for acute radiation syndrome is approximately 2 Gy for humans, approximately 3 Gy for an NHP model (e.g. rhesus monkey), and approximately 5 Gy for a mouse model.

Therefore, the methods of the present invention can be used to assess an absorbed dose of ionizing radiation in a patient sample by measuring levels of a combination of biomarkers in a sample and applying an algorithm to assess the absorbed dose in the sample based on the levels of the plurality of biomarker in the samples, wherein the combination of biomarkers comprise IL-15, CD5, Flt-3L, and salivary amylase. In embodiments, the algorithm quantifies an absorbed dose of ionizing radiation in the range of about 1-10 Gy, preferably between about 1-6 Gy, more preferably between about 2-6 Gy, or between about 6-10 Gy.

All or one or more parts of the algorithm(s) and statistical method(s) disclosed herein can be performed by or executed on a processor, general purpose or special purpose or other such machines, integrated circuits or by any combination thereof. Moreover, the software instructions for performing the algorithm(s) and statistical methods(s) disclosed herein may also be stored in whole or in part on a computer-readable medium, i.e., a storage device for use by a computer, processor, general or special purpose or other such machines, integrated circuits or by any combination thereof. A non-limiting list of suitable storage devices includes but is not limited to a computer hard drive, compact disk, transitory propagating signals, a network, or a portable media device to be read by an appropriate drive or via an appropriate connection.

In addition to biomarker measurements, radiation exposure assessment can benefit from additional inputs, such as information regarding clinical symptoms. For example, the Biodosimetry Assessment Tool (BAT) is a software application that equips healthcare providers with diagnostic information (clinical signs and symptoms, physical dosimetry, etc.) relevant to the management of human radiation casualties. Designed primarily for prompt use after a radiation incident, the software application facilitates the collection, integration, and archival of data obtained from exposed persons. Data collected in templates are compared with established radiation dose responses, obtained from the literature, to provide multi-parameter dose assessments. The program archives clinical information (extent of radioactive contamination, wounds, infection, etc.) useful for casualty management, displays relevant diagnostic information in a concise format, and can be used to manage both military and civilian radiation accidents.

In embodiments, the method further comprises, in response to a determination that the human has been exposed to radiation, administering an agent for treating radiation exposure in a human. Exemplary agents for treating radiation exposure include, but are not limited to, potassium iodide (KI), Prussian blue, diethylenetriamine pentaacetate (DTPA), and neupogen. Suitable treatments and regimens can be determined by the skilled artisan and are further described, e.g., in Wagner et al., Radiographics 14(2): 387-396 (1994); Kazzi et al., Emerg Med Clin North Am 33(1): 179-196 (2015); and Yamamoto, Pediatr Emerg Care 29(9): 1016-1026 (2013).

Kits

In embodiments, the present disclosure further provides a kit comprising, in one or more vials, containers, or components: (a) a surface comprising at least a first, second, third, and fourth binding reagent immobilized on an associated first, second, third, and fourth binding domain, wherein the first, second, third, and fourth binding reagent is a binding partner of IL-15, CD5, Flt-3L, and salivary amylase, respectively; (b) a detection reagent that specifically binds to biomarker IL-15; (c) a detection reagent that specifically binds to CD5; (d) a detection reagent that specifically binds to Flt-3L; and (e) a detection reagent that specifically binds to salivary amylase.

The biomarkers IL-15, CD5, Flt-3L, and salivary amylase, and binding and detection reagents therefor are described herein. In embodiments, each binding reagent and detection reagent is an antibody. In embodiments, each detection reagent comprises a detectable label. In embodiments, the detection reagent is lyophilized. In embodiments, the detection reagent is provided in solution. In embodiments, the binding reagents are immobilized on the binding domain. In embodiments, the binding reagents are provided in solution. In embodiments, the reagents and other components of the kit are provided separately. In embodiments, they are provided separately according to their optimal shipping or storage temperatures.

In embodiments, the surface of kit further comprises one or more additional binding reagents that are binding partners of CD20, IL-18, CD27, and/or TPO. In embodiments, the kit further comprises a detection reagent that specifically binds to CD20, a detection reagent that specifically binds to IL-18, a detection reagent that specifically binds to CD27, a detection reagent that specifically binds to TPO, or combination thereof. In embodiments, the surface of kit further comprises one or more additional binding reagents that are binding partners of CD20, and/or IL-18. In embodiments, the kit further comprises a detection reagent that specifically binds to CD20, a detection reagent that specifically binds to IL-18, or both.

Reagents and methods for immobilizing binding reagents to surfaces, e.g., via targeting agents/targeting agent complements, linking agents/supplemental linking agents, and bridging agents are described herein. In embodiments, the surface is a plate. In embodiments, the surface is a multi-well plate. In embodiments, the surface is a particle. In embodiments, the surface is a cartridge. In embodiments, the surface comprises an electrode. In embodiments, the electrode is a carbon ink electrode.

In embodiments, the surface comprises one or more binding reagent(s) described herein immobilized on one or more binding domains on the surface. In embodiments, the surface is an assay plate. In embodiments, the assay plate is provided in a vacuum sealed and/or desiccated secondary container, e.g., a foil pouch. In embodiments, the assay plate is removed from the vacuum sealed and/or desiccated secondary container and stored in open air (e.g., on a laboratory bench) prior to use in a method as described herein. In embodiments, the assay plate is stored at about 0° C. to about 40° C., about 2° C. to about 30° C., or about 4° C. to about 25° C. prior to use, e.g., in a method as described herein. In embodiments, the assay plate is stored for about 0 hours to about 80 hours, about 1 hour to about 60 hours, or about 2 hours to about 48 hours, or about 4 hours to about 24 hours, or about 8 hours to about 16 hours prior to use, e.g., in a method as described herein. In embodiments, the assay plate is stored for about 0 hours to about 72 hours at about 37° C. prior to use, e.g., in a method as described herein. In embodiments, storage of the assay plate in open air (e.g., for about 72 hours at about 37° C.) does not substantially vary the assay performance. In embodiments, storage of the assay plate in open air as described herein (e.g., for about 72 hours at about 37° C.) does not vary the biomarker measurements by more than 5%, 10%, 20%, 25%, or 30% as compared to an assay plate that was immediately used upon removal from its container, e.g., a vacuum sealed and/or desiccated secondary container. In embodiments, the assay plate is stable to storage for about 0 hours to about 72 hours at about 37° C. As used herein, “stable to storage” means that assay performance of the assay plate does not vary substantially after being subjected to a specified temperature and amount of time. In embodiments, an assay plate that is stable to storage provides biomarker measurements that do not vary by more than 5%, 10%, 15%, 20%, 25%, or 30% as compared to biomarker measurements from an assay plate that is used immediately upon removal from its container (e.g., a vacuum sealed and/or desiccated container). In embodiments, an assay plate that is stable to storage provides biomarker measurements that do not vary by more than 5%, 10%, 15%, 20%, 25%, or 30% as compared to biomarker measurements from an assay plate that is used after storage at about 2° C. to about 8° C.

In embodiments, the kit further comprises at least one non-immobilized competing reagent. Non-immobilized competing reagents for competing with a binding reagent for binding to its target biomarker are described herein. In embodiments, the non-immobilized competing reagent is an antibody. In embodiments, the non-immobilized competing reagent is lyophilized. In embodiments, the non-immobilized competing reagent is provided in solution. In embodiments, the non-immobilized competing reagent is a binding partner of salivary amylase. In embodiments, the non-immobilized competing reagent is lyophilized. In embodiments, the non-immobilized competing reagent is provided in solution.

In embodiments, the kit further comprises a calibration reagent, a control reagent, or both. In embodiments, the calibration reagent comprises a known quantity of a biomarker of interest, e.g., a known quantity of IL-15, CD5, Flt-3L, or salivary amylase. In embodiments, multiple calibration reagents comprise a range of concentrations of the biomarker. In embodiments, the multiple calibration reagents comprise concentrations of a biomarker near the upper and lower limits of quantitation for the immunoassay. In embodiments, the multiple concentrations of the calibration reagent spans the entire dynamic range of the immunoassay. In embodiments, the control reagent comprises a sample obtained from an individual not exposed to radiation. In embodiments, the control reagent is used to provide a basis of comparison for the biological sample to be tested with the methods of the present disclosure. In embodiments, the calibration reagent, the control reagent, or both, are lyophilized. In embodiments, the calibration reagent, the control reagent, or both, are provided in solution.

In embodiments, the calibration reagent is lyophilized, reconstituted, and stored prior to use in a method as described herein. In embodiments, the reconstituted calibration reagent is stored for about 0 hours to about 48 hours, about 5 minutes to about 36 hours, about 15 minutes to about 24 hours, about 30 minutes to about 12 hours, about 2 hours to about 9 hours, about 4 hours to about 8 hours, or about 5 hours to about 6 hours prior to use in a method as described herein. In embodiments, the reconstituted calibration reagent is stored at about 0° C. to about 30° C., about 2° C. to about 28° C., or about 4° C. to about 25° C. In embodiments, the reconstituted calibration reagent is stored at about 4° C. for about 0 hours to about 24 hours. In embodiments, the reconstituted calibration reagent is stored at about 25° C. for about 0 hours to about 6 hours.

In embodiments, the control reagent is lyophilized, reconstituted, and stored prior to use in a method as described herein. In embodiments, the reconstituted control reagent is stored for about 0 hours to about 48 hours, about 5 minutes to about 36 hours, about 15 minutes to about 24 hours, about 30 minutes to about 12 hours, about 2 hours to about 9 hours, about 4 hours to about 8 hours, or about 5 hours to about 6 hours prior to use in a method as described herein. In embodiments, the reconstituted control reagent is stored at about 0° C. to about 30° C., about 2° C. to about 28° C., or about 4° C. to about 25° C. In embodiments, the reconstituted control reagent is stored at about 4° C. for about 0 hours to about 24 hours. In embodiments, the reconstituted control reagent is stored at about 25° C. for about 0 hours to about 8 hours.

In embodiments, reconstitution and storage of the calibration reagent as described herein (e.g., for about 24 hours at 4° C. or for about 6 hours at 25° C.) does not substantially vary its assay performance. In embodiments, reconstitution and storage of the calibration reagent does not vary the biomarker measurements by more than 5%, 10%, 20%, 25%, or 30% as compared to a calibration reagent that is immediately used upon reconstitution. In embodiments, reconstitution and storage of the control reagent as described herein (e.g., for about 24 hours at 4° C. or for about 8 hours at 25° C.) does not substantially vary its assay performance. In embodiments, reconstitution and storage of the control reagent does not vary the biomarker measurements by more than 5%, 10%, 20%, 25%, or 30% as compared to a control reagent that is immediately used upon reconstitution.

In embodiments, the kit further comprises a diluent for one or more of the various reagents in the kit. In embodiments, the diluent is subjected to heat during manufacture. In embodiments, the diluent is subjected to a temperature of about 50° C. to about 80° C., about 55° C. to about 75° C., about 60° C. to about 70° C., about 61° C. to about 65° C., or about 62° C. to about 64° C. during manufacture. In embodiments, heat treatment of the diluent reduces interference and/or non-specific binding when performing assays with the kit components.

In embodiments, the kit further comprises one or more of a buffer, e.g., assay buffer, reconstitution buffer, storage buffer, read buffer, and the like; an assay consumable, e.g., assay modules, vials, tubes, liquid handling and transfer devices such as pipette tips, covers and seals, racks, labels, and the like; an assay instrument; and/or instructions for carrying out the assay.

In embodiments, the kit comprises lyophilized reagents, e.g., detection reagent, non-immobilized competing reagent, calibration reagent, and control reagent. In embodiments, the kit comprises one or more solutions to reconstitute the lyophilized reagents.

In embodiments, a kit comprising the components above include stock concentrations of the components that are 5×, 10×, 20×, 30×, 40×, 50×, 60×, 70×, 80×, 90×, 100×, 125×, 150× or higher fold concentrations of the concentrations (e.g., coating, working, calibration, and control concentrations) set forth above.

All references cited herein, including patents, patent applications, papers, textbooks and the like, and the references cited therein, to the extent that they are not already, are hereby incorporated herein by reference in their entirety.

### Examples

A list of candidate radiation biomarkers is shown below in Table 1. All of the biomarkers listed in Table 1, with the exception of salivary amylase, are sufficiently homologous between humans and an nonhuman primate (NHP) (rhesus monkey) such that the same assay can be used for both species. Due to large differences in the isoforms of amylase produced by the salivary gland in humans (AMY1A) and rhesus (AMY1B), separate salivary amylase assays were developed for the two species.

TABLE 1

| Mechanism of | | |
|---|---|---|
| Radiation Response | Biomarker | Comments |
| Hematopoietic | CD5 | T cell surface marker |
| Damage Markers | CD20 | B cell surface marker |
| | CD27 | Lymphocyte surface marker |
| | CD 177 | Neutrophil surface marker |
| Hematopoietic Repair | Flt-3L | Hematopoietic progenitors |
| Factors and | EPO | Erythrocytes |
| Cytokines | TPO | Platelets |
| | IL-12 | Pro-inflammatory cytokine |
| | IL-15 | Cells of innate immune system |
| | IL-18 | Pro-inflammatory cytokine |
| Acute Phase Proteins | CRP | Acute phase response |
| Salivary Gland | Salivary Amylase | Human marker |
| | (AMY1A) | |
| Damage Marker | Salivary Amylase | NHP marker |
| | (AMY2A) | |

Assays described herein are immunoassays utilizing a capture antibody and a detection antibody for each of the biomarkers described herein. Antibodies and calibrators for CD20, IL-15, AMY1A, CD5, and FLT-3L were obtained from Sinobiologicals, Origene, R&D Systems, Diaclone (Sapphire North America), Roche, Cell Sciences, and MSD.

Unless described otherwise, plates for the assays described herein are pre-coated multi-well plates. Calibrators were a blend of Cal-1 through Cal-8, each containing different known concentrations of each of the biomarkers in a particular panel. Detection antibodies were conjugated to MSD's SULFO-TAG label according to known methods. Capture antibodies were conjugated with biotin according to known methods. External controls include a negative control, i.e., calibration levels reflect negative radiation results (<2 Gy), and a positive control, i.e., calibration levels reflect positive radiation results (≥2 Gy).

Unless described otherwise, the multiplexed assay is performed as follows:
(1) couple linker to capture antibody;
(2) vortex and incubate 30 minutes;
(3) add stop solution, vortex, and incubate 30 minutes;
(4) mix all capture antibody-linkers in solution;
(5) coat plate with capture antibody solution, incubate at room temperature for 1 hour with shaking;
(6) wash plate;
(7) dispense 25 μL detection antibody and 25 μL calibrator or sample;
(8) incubate for 1 hour at room temperature with shaking and wash;
(9) add 150 μL/well of ECL assay read buffer;
(10) read assay plate immediately.

The biomarkers in Table 1 were evaluated using assays described in embodiments herein. Details and results of these assays are further described in the following Examples.

### Example 1. Desensitization of Assay for Human Salivary Amylase

To generate a single assay panel with multiple biomarkers, it was necessary to combine assays for biomarkers that should be run without dilution to achieve a desired sensitivity (e.g., low abundance markers such as CD20) with assays for biomarkers that should be diluted due to the high levels present in plasma (e.g., high abundance markers such as salivary amylase). An assay desensitization was developed by adding non-immobilized (“free”) capture antibody to the assay reaction mixture, which competed with the immobilized capture antibody for the biomarker, thereby providing the desired assay dynamic range.

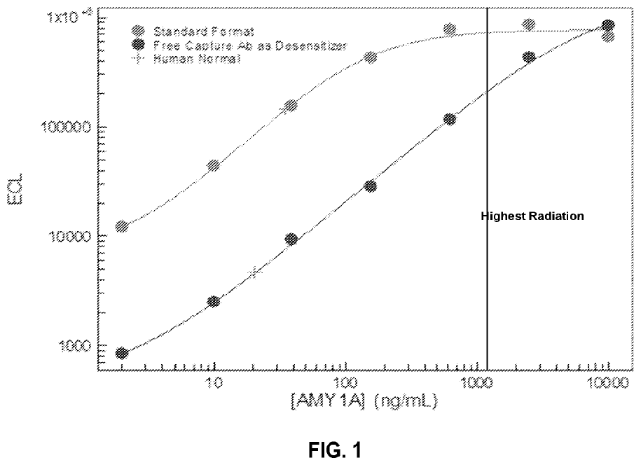

The assay for salivary amylase AMY1A was desensitized by adding non-immobilized capture antibody for AMY1A to the assay reaction mixture. Calibration curves of standard and desensitized assay formats are shown in FIG. 1. In the standard format (light grey), dynamic range was not achieved between normal (cross) and irradiated (vertical line) individuals, while in the desensitized format (dark grey), the calibration curve was linear in the range between normal and irradiated individuals, indicating the desired dynamic range was achieved.

### Example 2. Calibration and Parameters of Multiplexed Biomarker Assays

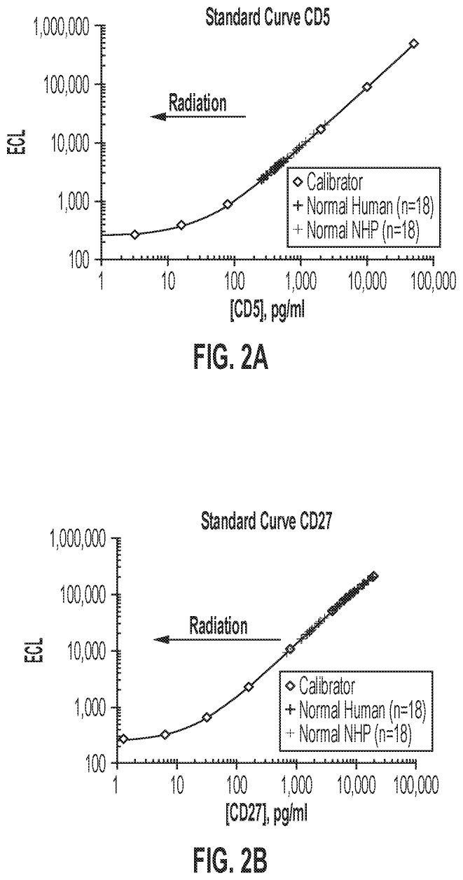

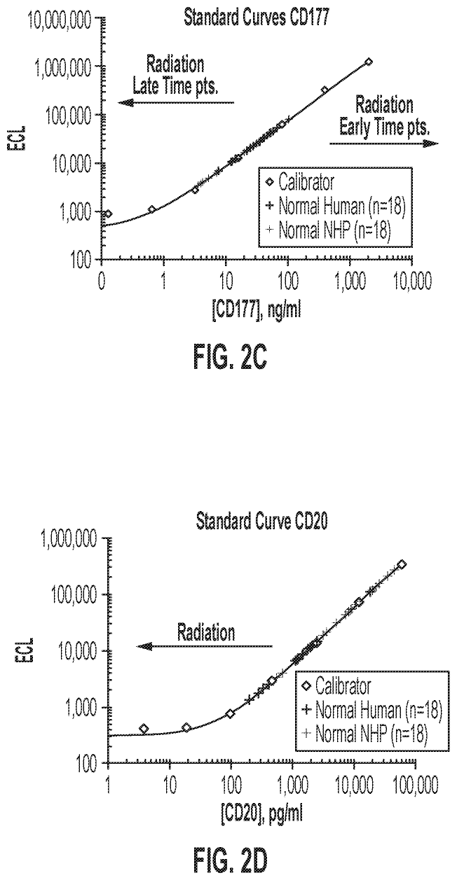

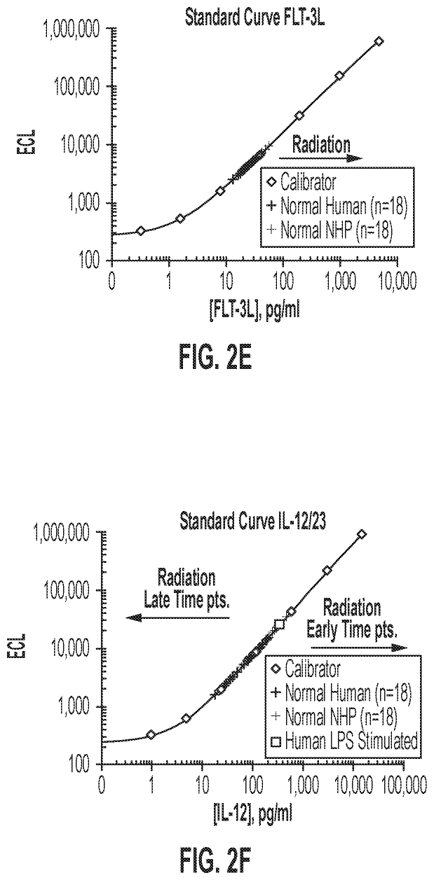

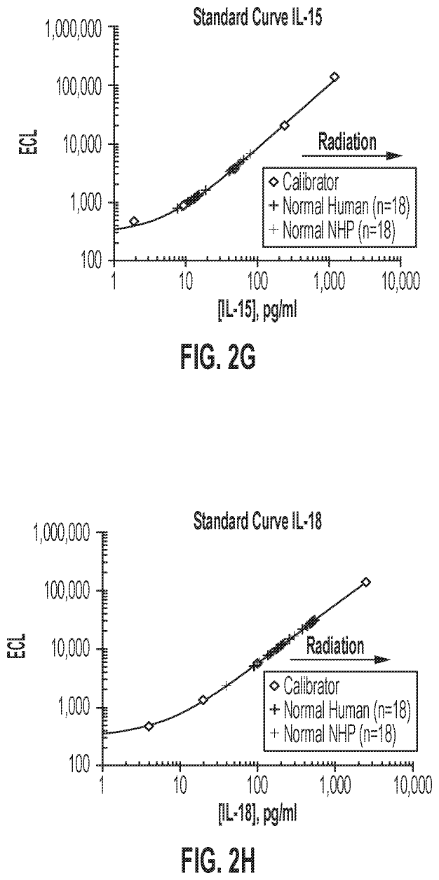

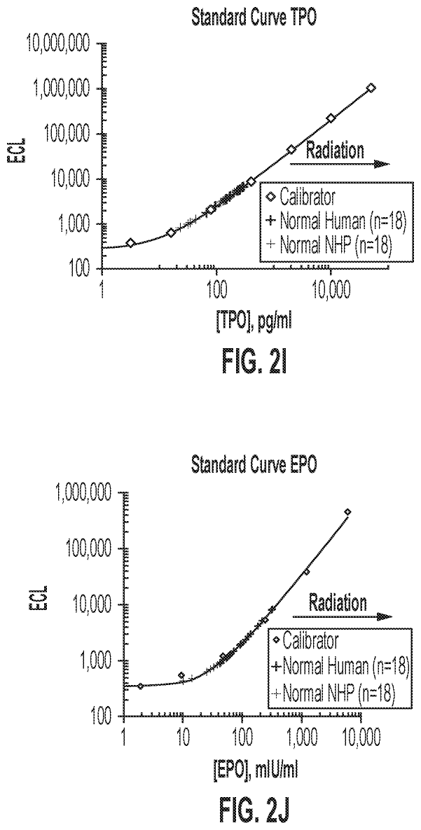

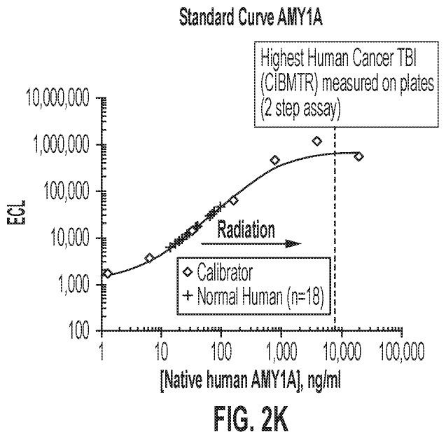

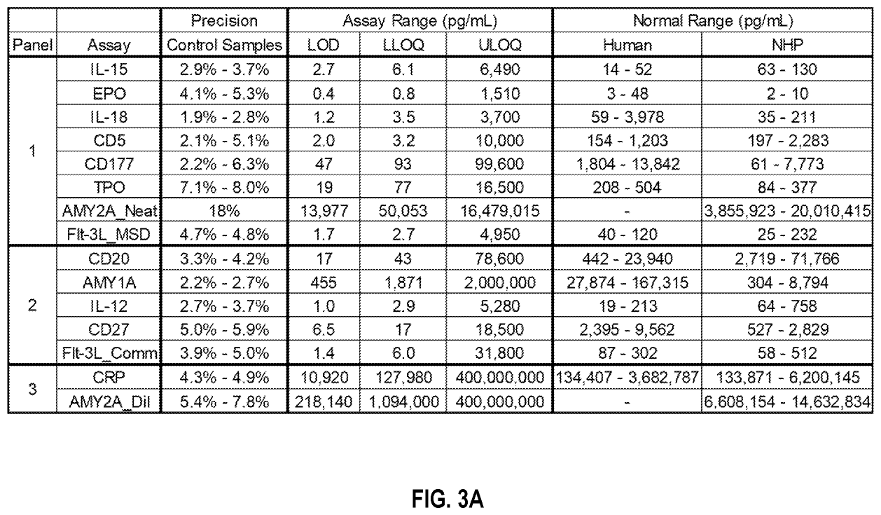

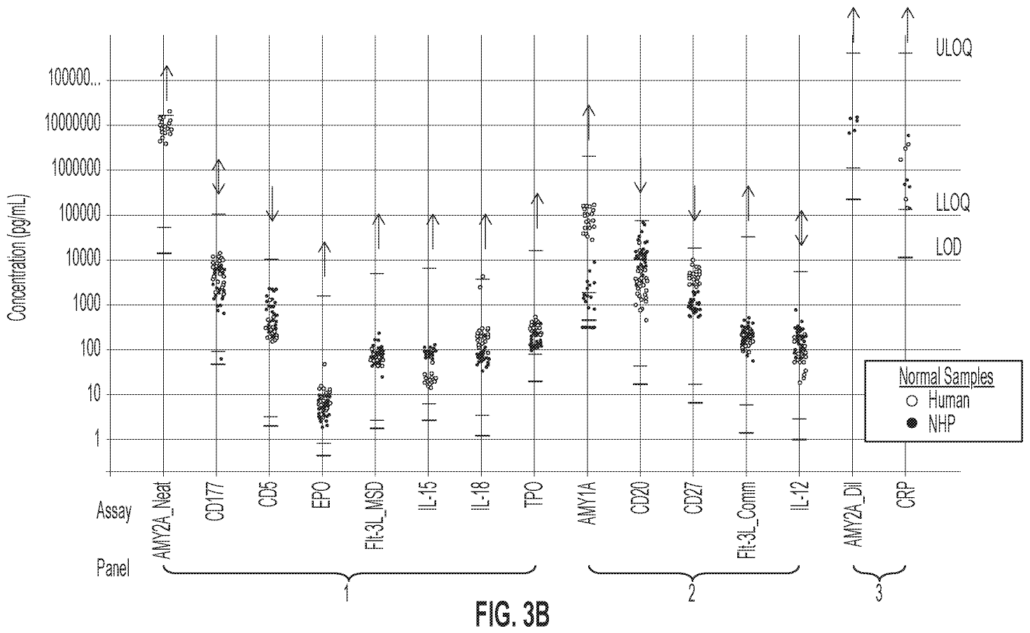

Biomarker assays for the biomarkers in Table 1 were run in multiplexed panels as shown in FIG. 3A. Assay calibration curves were generated for the biomarkers in Table 1. FIGS. 2A-2J show, respectively, the calibration curves for CD5, CD27, CD177, CD20, Flt-3L, IL-12/23, IL-15, IL-18, thyroid peroxidase (TPO), erythropoietin (EPO), and AMY1A. Measured levels for a set of normal plasma samples from 18 human donors (bold-lined cross) and 18 NHP (thin-lined cross) are superimposed on the curves. Arrows in each graph represent the direction to which the curve is expected to shift after exposure to radiation. As shown in the calibration curves in FIGS. 2A-2K, each of the assays are capable of measuring normal levels and have available dynamic range to measure changes after radiation exposure.

Assay control samples containing 8 replicates were measured over 2 assay plates, and 32 normal (non-irradiated) human plasma samples and 31 normal (non-irradiated) NHP plasma samples were measured using the multiplexed biomarker panels indicated in FIG. 3A. FIG. 3A shows the coefficient of variation (column labeled “Precision”) measured for the two assay control samples, assay measurement range (limit of detection (LOD), lower limit of quantitation (LLOQ), and upper limit of quantitation (ULOQ)), and range of biomarker concentration values measured for the human and NHP plasma samples. The LOD is set as the concentration that provides a signal 2.5 standard deviations above a blank sample; the LLOQ and ULOQ are the lowest and highest concentrations, respectively, that can be measured with imprecision and inaccuracy of less than or equal to 20%.

Results of the measured biomarker concentrations in the human and NHP plasma samples relative to the LOD (bold-lined bars), LLOQ and ULOQ (thin-lined bars) are shown in FIG. 3B. Arrows above each column represent the direction in which the concentration is expected to change after exposure to radiation. Samples that were undetectable, if any, were assigned a value of (⅔)×LOD, such that their values can be displayed on the plot just under the LOD bar. The assays labeled “Flt-3L_MSD” and “Flt-3L_Comm” are both Flt-3L assays but utilize different antibody pairs. The assay labeled “AMY2A_Neat” is a desensitized AMY2A assay as described above (i.e., without dilution). The assay labeled “AMY2A_Dil” is an AMY2A assay that was performed with CRP in a diluted, standard format.

As shown in FIGS. 3A and 3B, most of the assays provided a range of quantitation that includes the normal samples and provides room for the expected change in levels after radiation, with the exceptions of: (1) AMY1A, which had the appropriate range for human samples but did not detect normal amylase levels in some normal NHP samples; and (2) the desensitized AMY2A assay (“AMY2A_Neat”), which is near the top of the quantitation range for normal NHP samples.

### Example 3. Linearity-On-Dilution and Spike Recovery Assessment

Linearity-on-dilution and spike recovery are assessment methods for validating and assessing the accuracy of an assay, e.g., identify sample matrix sensitivities and/or determine if the calibration approach is appropriate. Linearity on dilution refers to the extent in which a spike or natural sample's (in a particular diluent) dose response is linear and in the desired assay range. Spike recovery is used to determine whether analyte detection is affected by differences in the standard curve diluent and biological sample matrix (e.g., an undiluted biological sample or a mixture of the biological sample with the matrix).

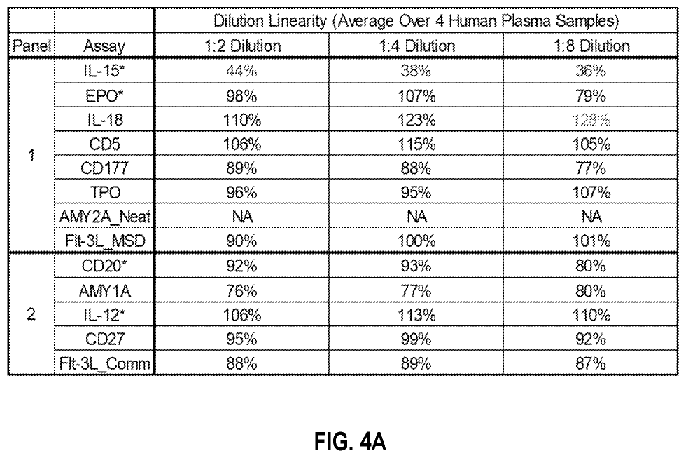

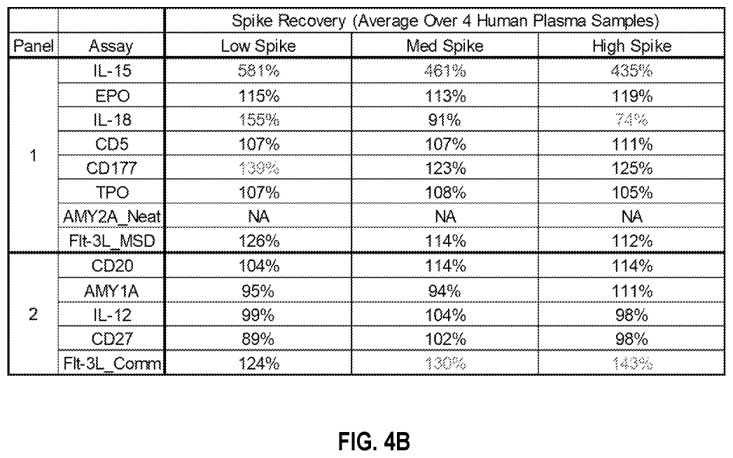

Linearity-on-dilution and spike recovery were assessed for the multiplexed biomarker panels shown in FIGS. 4A and 4B. Linearity-on-dilution was assessed for normal plasma samples diluted into assay calibrator diluent. FIG. 4A reports the measured concentration as a percentage of the expected concentration, based on the measured concentration prior to dilution. Analytes marked with asterisk (*) had normal levels near the LLOQ, and thus purified calibrator was added prior to performing the experiment.

Spike recovery was assessed for a purified calibrator biomarker spiked into plasma samples. FIG. 4B reports the measured increase in concentration due to the spike, as a percentage of the expected increase. In FIGS. 4A and 4B, each value represents the average recovery value across four different plasma samples.

As shown in FIGS. 4A and 4B, most of the biomarkers have recovery values within a target range of 75% to 125%. A notable exception was IL-15, which had very low values on dilution and very high values in spike recovery.

### Example 3.1. Diluent Treatment

As discussed above in Example 3, most of the biomarkers in FIGS. 4A and 4B have recovery values within a target value of 75% to 125%, with the exception of IL-15. Further investigation determined that this effect observed with IL-15 may be due to an interferent in the calibration diluent that decreases the signal for the calibration standard relative to the analyte in the serum matrix.

The calibrator diluent (MSD Diluent 6) was subjected to heat treatment. Briefly, the diluent was thawed overnight at 4° C. The thawed diluent was incubated in a water bath at 62 to 64° C. for 60 minutes, with mixing every 5 minutes for equal heat distribution. The heat treated diluent was then cooled on ice, with mixing every 10 minutes for up to 30 to 40 minutes. Trehalose was added (to low final concentration) and mixed for at least 30 minutes. The diluent was then dispensed into designated bottles and frozen on dry ice. The heat inactivated calibrator appeared to mitigate the interferent effect.

### Example 4. Nonhuman Primate (NHP) Radiation Study

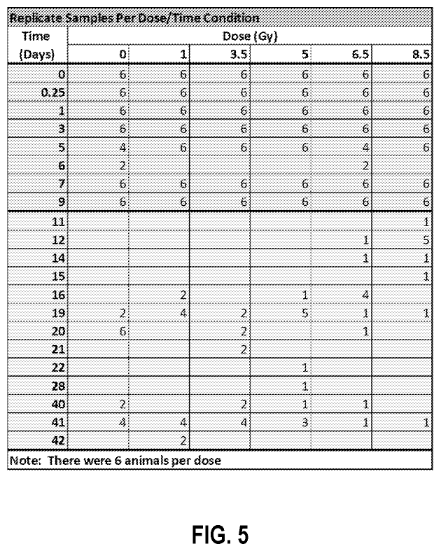

An NHP radiation study was performed to characterize the biomarkers and assess the multiplexed assay panels described in Examples 1-3. FIG. 5 shows a summary of the samples used in the NHP radiation study. Six animals were exposed to each dose condition shown in FIG. 5. Plasma samples were collected at different time points before (0 days) and after radiation. The numbers in FIG. 5 indicate the number of samples tested for each dose-time combination.

### Example 4.1 Hematopoietic Damage Markers

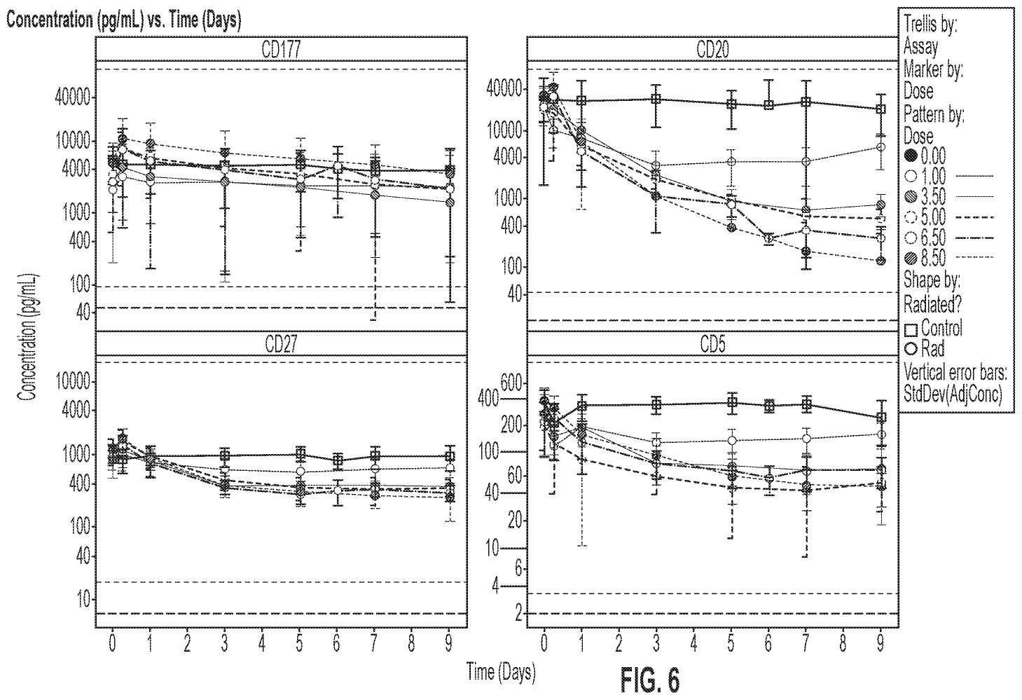

FIG. 6 shows changes in lymphocyte (CD5, CD20, CD27) and neutrophil (CD177) surface biomarkers in NHP plasma as a function of time (for the first 9 days) and radiation dose. Error bars represent the standard deviation in the measured biomarker level across the different replicate animals. The lowest horizontal line in each plot represents the assay LOD. The upper and middle horizontal lines in each plot represents the quantitation range defined by the LLOQ (middle line) and ULOQ (upper line).

As shown in FIG. 6, all three lymphocyte markers showed a decrease in levels with radiation and time for all doses. In particular, CD5 and CD20 levels in irradiated animals were clearly separated from levels in negative control animals even as early as one day after total body irradiation (TBI). CD27 showed a short spike at an early time, then decreased below the negative control level.

### Example 4.2 Hematopoietic Repair Factors and Cytokines

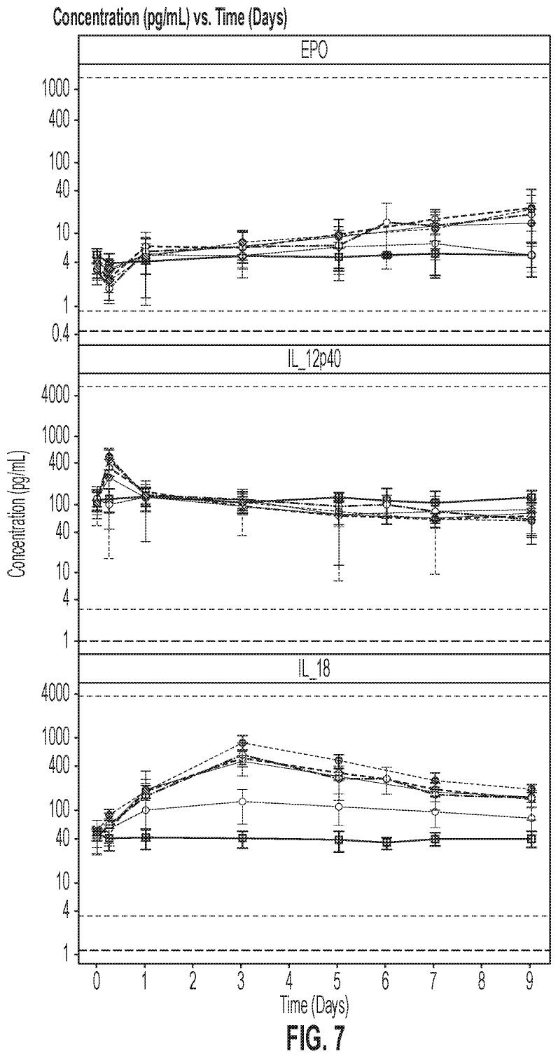

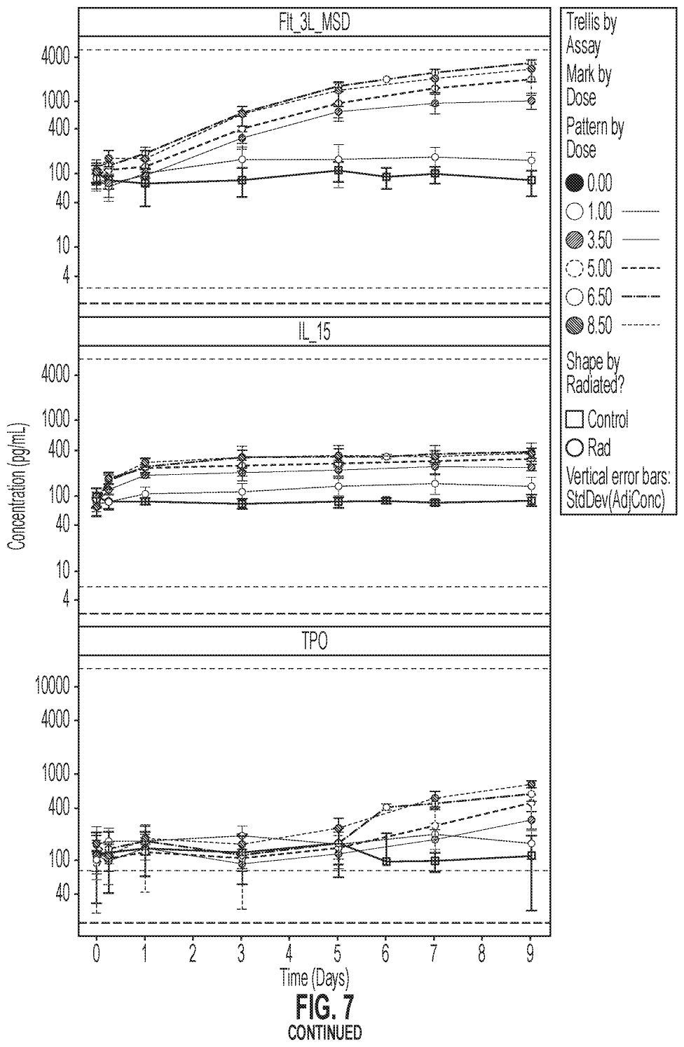

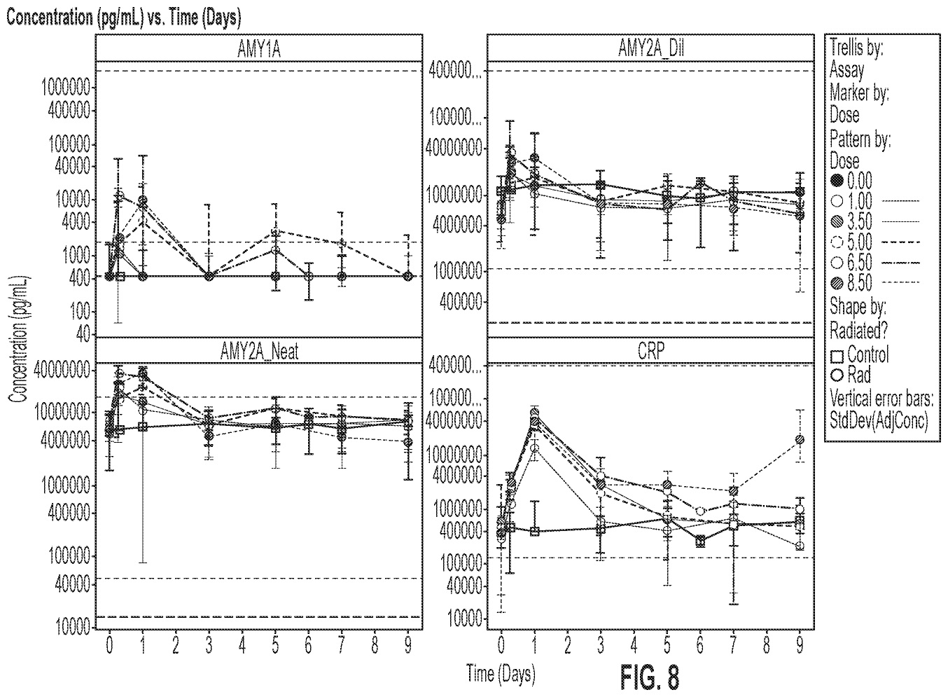

FIG. 7 shows changes in growth factor and cytokine biomarkers (IL-12, IL-15, IL-18, Flt-3L, EPO, and TPO) in NHP plasma as a function of time (for the first 9 days) and radiation dose. Error bars and LOD, LLOQ and ULOQ lines are indicated in the same manner as in Example 4.1.

As shown in FIG. 7, IL-15, IL-18, and Flt-3L all showed a significant increase in levels with from day one post-TBI. Elevations in EPO and TPO were not observed until nearly one week post-TBI, with changes in EPO being relatively small. Similar results for Flt-3L were obtained using a different antibody pair (“Flt-3L_Comm”) as shown in FIG. 3B.

### Example 4.3 Salivary Gland Damage Marker and Acute Phase Proteins

FIG. 8 shows changes in salivary amylase (AMY1A, AMY2A measured using a desensitized assay in undiluted (neat) samples and AMY2A also measured in diluted samples) in NHP plasma as a function of time (for the first 9 days) and radiation dose. Error bars and LOD, LLOQ and ULOQ lines are indicated in the same manner as in Example 4.1.

As shown in FIG. 8, the human salivary amylase (AMY1A) assay was not sensitive enough to measure changes at lower dose levels. The NHP salivary amylase (AMY2A) desensitized assay performed with undiluted samples showed the expected increase in levels at early time points but did not have the dynamic range to accurately quantitate the levels, as many samples were above the ULOQ for the assay. The desensitized AMY2A assay showed more significant separation between the irradiated and control samples compared with the standard format AMY2A assay. C-reactive protein (CRP) provided an expected large spike in the first 1 to 3 days post-TBI and remained elevated for the higher doses.

### Example 4.4 Alternative Dosing Regimen for NHP Study

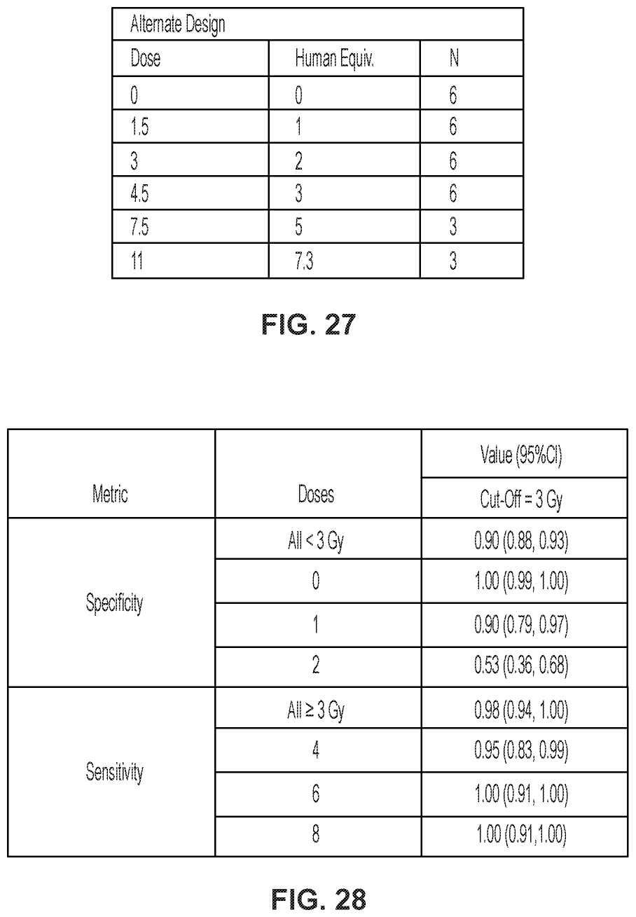

An alternative dosing regimen to the one shown in FIG. 5 can be seen in FIG. 27. The alternative dosing regimen includes a dose of 3 Gy, which is equivalent to a 2 Gy dose in humans. The alternative dosing regimen would include 6 animals per dose for all doses except for 7.5 Gy and 11 Gy, which would include 3 animals each. The inclusion of some high dose samples would demonstrate that the algorithm is unlikely to hook at high doses.

### Example 5. Stem Cell Transplant (SCT) Patient Study

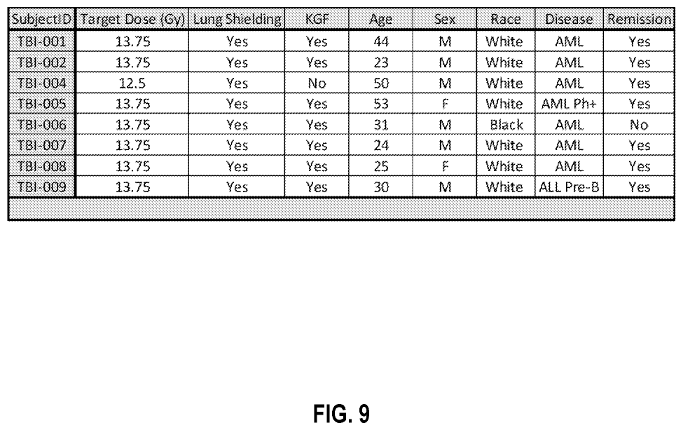

A stem cell transplant (SCT) patient study was performed to characterize the biomarkers and assess the multiplexed assay panels described in Examples 1-3. FIG. 9 shows a summary of the patients in the study. Study subjects were cancer patients (either acute myeloid leukemia (AML) or acute lymphoblastic leukemia (ALL)) receiving myeloablative radiation prior to stem cell transplant, with all but one of the patients in remission at the time of radiation.

There was a period of at least three weeks between the last round of induction chemotherapy and the start of total body irradiation (TBI) (Day 0). All patients received the target dose indicated in FIG. 9 in 1.25 Gy fractions, with 2 to 3 fractions per day over 4 days. All patients received lung shielding and e-beam boost to chest, and most patients received keratinocyte growth factor (KGF).

Sampling was performed on Day 0 (pre-TBI), 1, 2, 3, 4, and 7 (post-chemotherapy). A sample was not obtained on Day 4 from all patients, and all patients received myeloablative chemotherapy on Day 4, and thus samples from Day 7 may be confounded by the chemotherapy treatment. The samples were archived from a previous study at Memorial Sloan Kettering and used in this study.

### Example 5.1 Hematopoietic Damage Markers

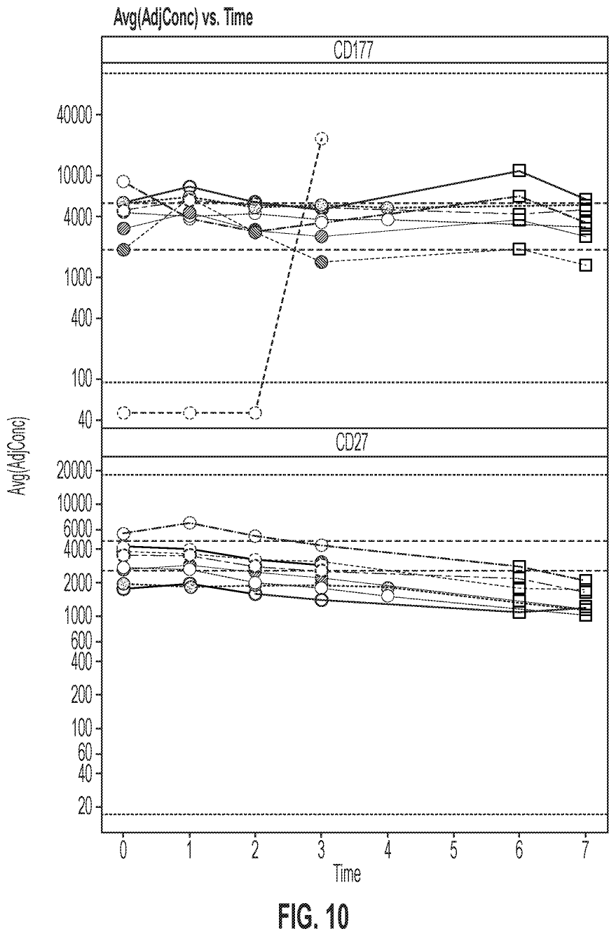

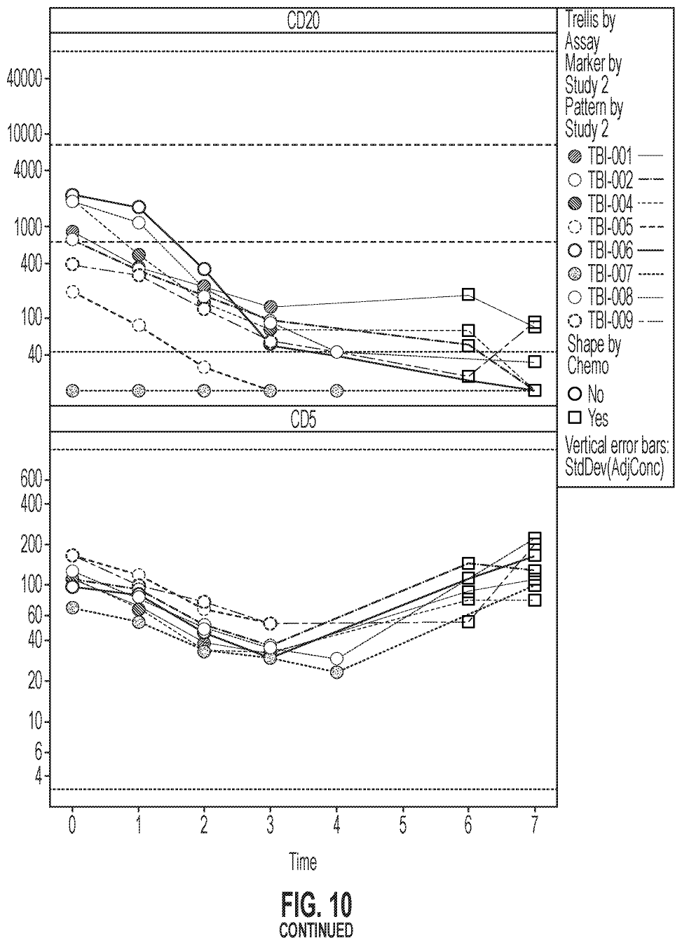

FIG. 10 shows changes in lymphocyte (CD5, CD20, and CD27) and neutrophil (CD177) surface biomarkers in human plasma from SCT patients as a function of time during fractionated TBI regimen. Each curve represents samples from a different patient. Points later than Day 4 may be confounded by chemotherapy as discussed above. The two horizontal dashed lines near the top and bottom of each plot provide the quantitation range defined by the LLOQ (lower line) and ULOQ (upper line). The two horizontal lines in the middle of each plot represent the ±1 standard deviation range for a set of 10 normal human plasma samples tested at the same time as the SCT patient samples.

As shown in FIG. 10, CD5 and CD20 showed a large decrease in levels after radiation. CD27 also decreases, but to a much lesser extent than was observed in the NHP model (see FIG. 6). CD177 was relatively unaffected by the fractionated dose regimen in humans.

As discussed herein, low baseline levels of CD20 in SCT patients can complicate its study. Thus, CD5 may be a promising biomarker as its levels decreased in both NHP (see Example 4.1) and humans, and baseline CD5 levels in SCT patients are roughly normal.

### Example 5.2 Hematopoietic Repair Factors and Cytokines

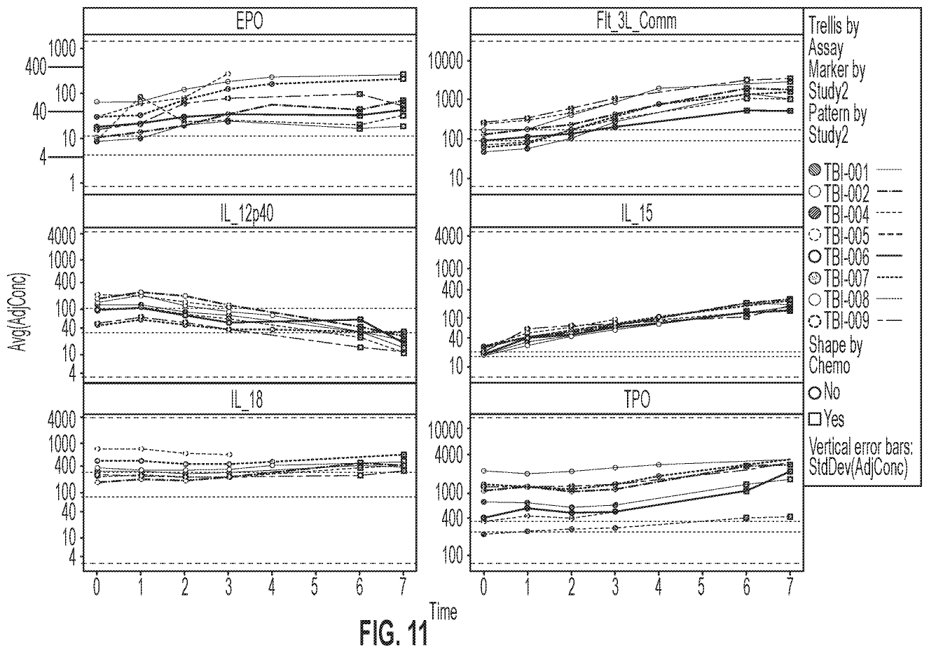

FIG. 11 shows changes in growth factor and cytokine biomarkers (IL-12, IL-15, IL-18, Flt-3L, EPO, and TPO) in human plasma from SCT patients as a function of time during fractionated TBI regimen. Each curve represents samples from a different patient. LLOQ, ULOQ, and standard deviation of normal plasma sample lines are indicated in the same manner as in Example 5.1.

As shown in FIG. 11, IL-15 and Flt-3L both increased significantly after radiation exposure, as was similarly observed for NHP (see Example 4.2). IL-18 in SCT patient samples did not increase in a similar manner as in the NHP model. Further studies may be needed to determine whether the discrepancy in IL-18 results between humans and NHP is due to differences in the IL-18 responses between the two species, or the use of fractionated doses in human patients, or some other biological difference associated with cancer or prior chemotherapy. TPO and EPO are late radiation markers, and thus the lack of a significant response in SCT patients was expected.

### Example 5.3 Salivary Gland Damage Marker and Acute Phase Proteins

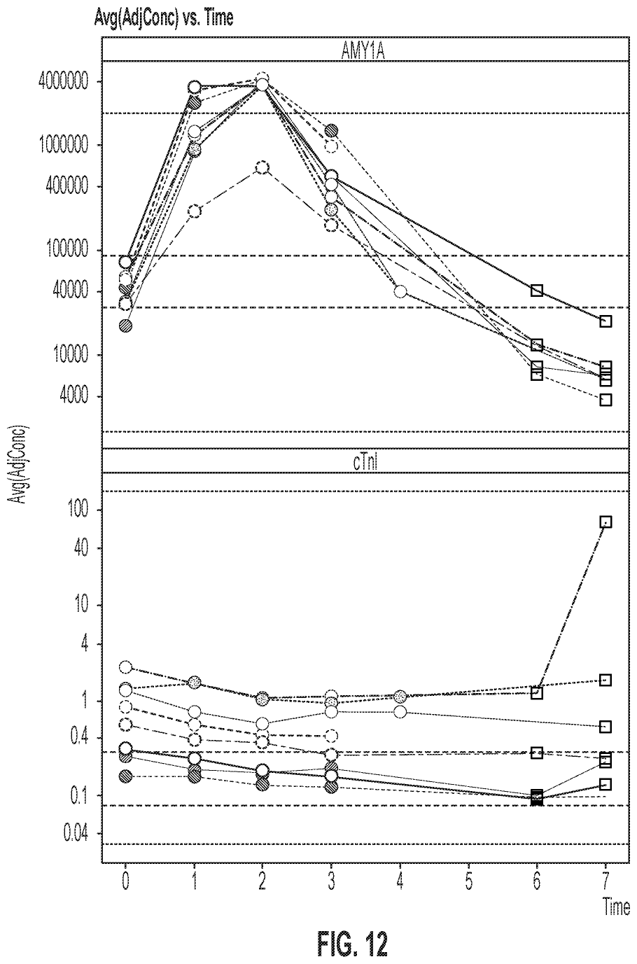

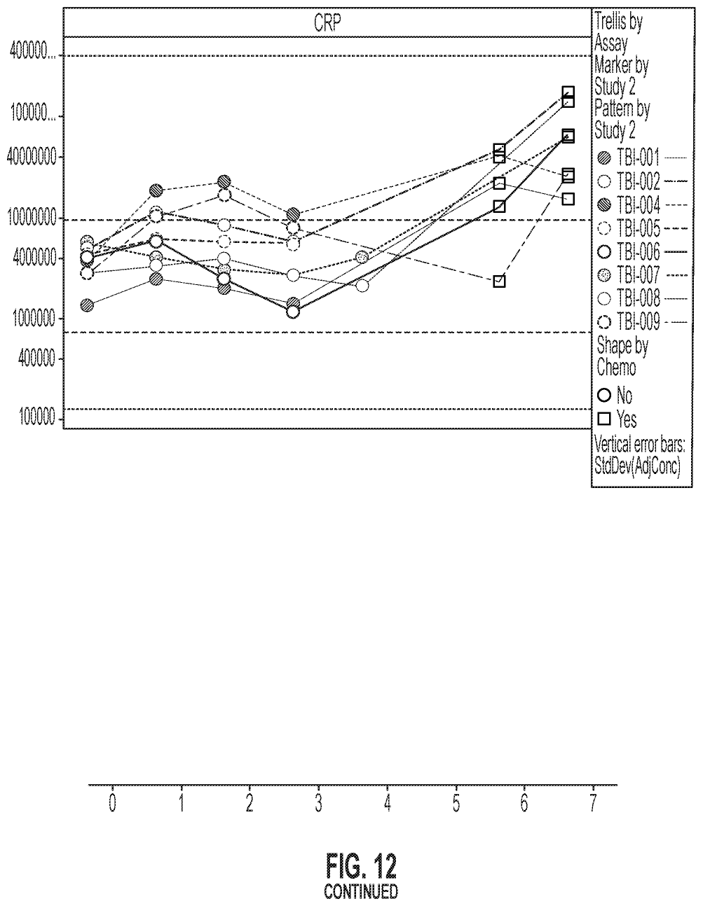

FIG. 12 shows changes in salivary amylase (AMY1A), C-reactive protein (CRP), and cardiac troponin (cTnl) in human plasma from SCT patients as a function of time during fractionated TBI regimen. Each curve represents samples from a different patient. LLOQ, ULOQ, and standard deviation of normal plasma sample lines are indicated in the same manner as in Example 5.1.

As shown in FIG. 12, amylase showed a strong increase at early time points (1 to 3 days) after TBI. The change in CRP was unexpectedly small, possibly due to the use of fractionated doses. No response was observed with cTnl, a marker of damage to heart muscle.

### Example 6. Determination of Radiation Dose from Biomarker Concentration

Radiation dose can be determined based on the measured concentrations of biomarkers described herein, e.g., using the algorithms described herein. The measured and fit values for the biomarker levels may be added linearly or in quadrature to the limit of detection (LOD) or lower limit of quantitation to minimize the effect on the cost function of changes in levels near to the detection limit as in the function below:

$$F ( dose ) = {\sum }_{i = 1}^{n} \vert log ( \frac{{m}_{i} + {LOD}_{i}}{{M}_{i} ( dose ) + {LOD}_{i}} ) \vert ,$$

wherein mᵢ is the measured value for biomarker i, M is the predicted biomarker value as a function of dose at a known time post-exposure, LOD, is the assay Limit of Detection for biomarker i, and n is the total number of biomarkers being used.

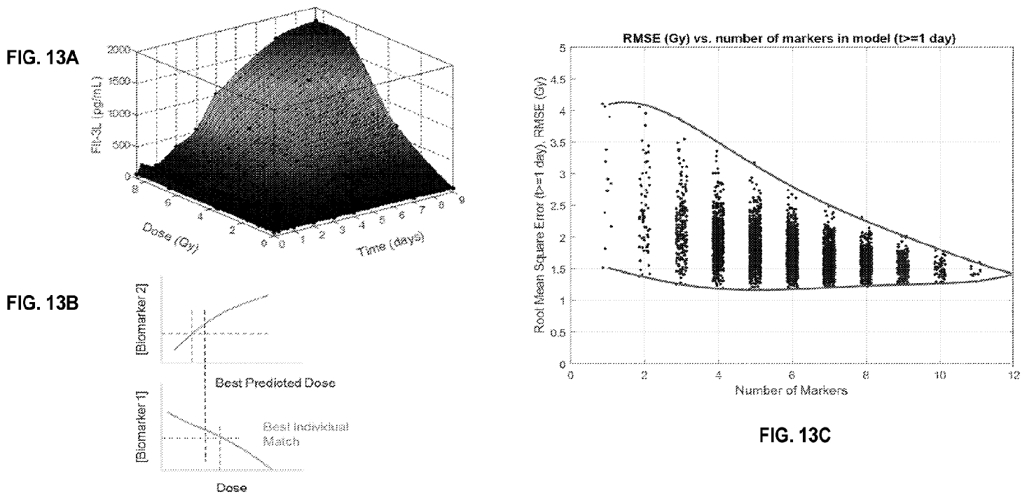

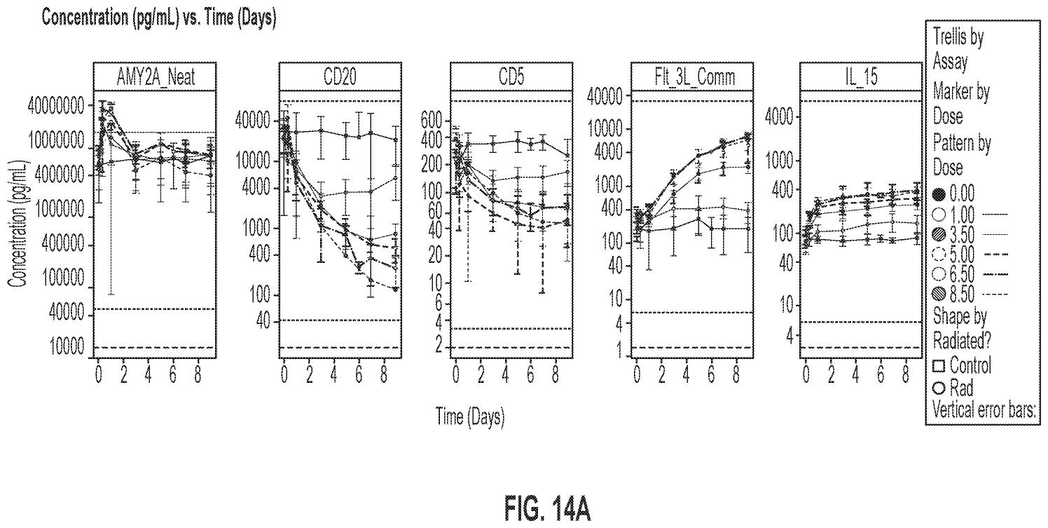

A training data set can be plotted as the average biomarker concentration vs. dose and time for each biomarker, for example, as illustrated in FIG. 13A. FIG. 13B shows an exemplary prediction model based on measured concentrations of two biomarkers, wherein the best predicted dose falls between the best individual matches for each of the two biomarkers. FIG. 13C shows the root mean square error (RSME) in dose prediction across all test samples for all possible combinations of biomarkers, i.e., each point represents the predictive ability obtained by combining results from a specific set of biomarkers. The plot indicates that the combination of four or more biomarkers provides a good prediction for radiation dose.

### Example 7. Five-Biomarker Panel Assays

A five-biomarker panel was prepared for both NHP and humans, which included the biomarkers from previous Examples that showed the best radiation responses. The NHP biomarker panel included AMY2A (desensitized assay format), CD20, CD5, Flt-3L, and IL-15. The human biomarker panel included AMY1A, CD20, CD5, Flt-3L, and IL-15.

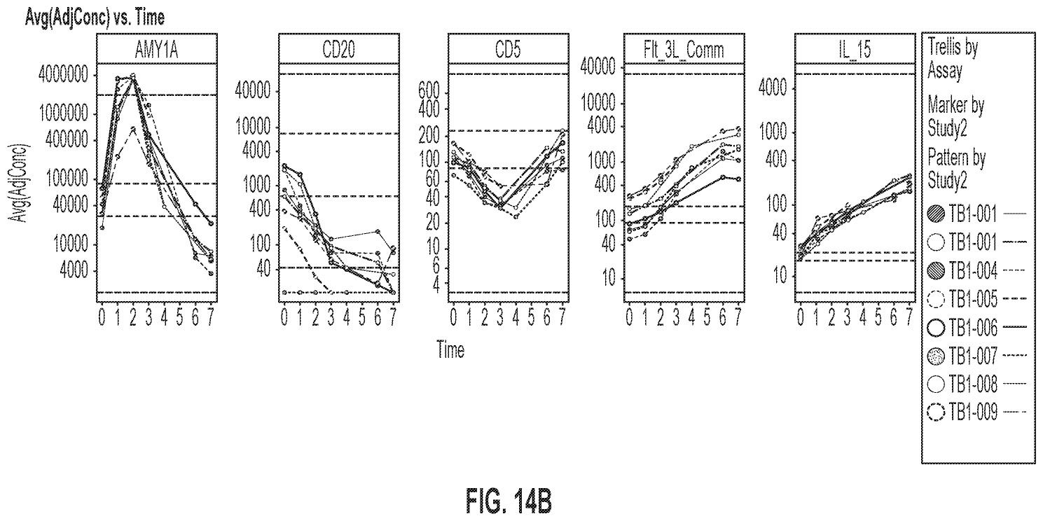

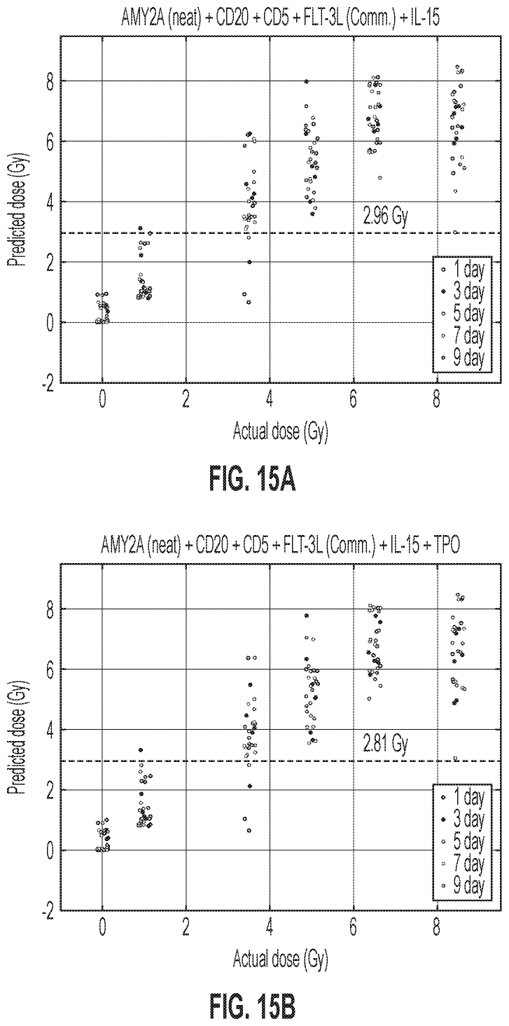

The NHP and human plasma samples described in Examples 4 and 5 were tested with the five-biomarker panel, with results of the measured biomarker concentrations shown in FIGS. 14A (NHP) and 14B (human). In the plots of FIG. 14A, the error bars and LOD, LLOQ and ULOQ lines are indicated in the same manner as in Example 4.1. In the plots of FIG. 14B, the curves, LLOQ, ULOQ, and standard deviation of normal plasma sample lines are indicated in the same manner as in Example 5.1.

FIG. 15A shows a plot of the predicted vs. actual radiation doses for the NHP samples tested with the five-biomarker panel. FIG. 15B shows a plot of the predicted vs. actual radiation doses for NHP samples tested with the same five-biomarker panel plus TPO. The points are indicated with different patterns by time post-TBI, and only the time points from 1 to 9 days post-TBI were analyzed. It can be seen from the plots that the five- or six-biomarker panels achieved good separation of the NHP exposed to above and below 3 Gy (dotted horizontal line).

### Example 8. Manual and Ultra-High Throughput Assay Development

A manual protocol was developed for an electrochemiluminescence (ECL) detection assay using a five-biomarker panel (AMY1A, CD5, CD20, Flt-3L, and IL-15) in a 96-well plate, as follows:

(1) In an assay well, combine 25 μL plasma sample+25 μL detection antibody mix;

(2) Incubate 60 minutes with shaking;

(3) Wash thoroughly to remove excess sample and/or detection antibody;

(4) Add ECL read buffer and analyze with ECL plate reader.

An ultra-high throughput (UHT) automated system was also developed to perform up to 20 assay plates in parallel. The UHT system is capable of processing a batch of 20 plates containing 760 samples in duplicate, producing first results in approximately 70 minutes and last results in approximately 130 minutes. Thus, the UHT system has a throughput of 1,520 sample wells in 130 minutes and is capable of processing more than 10,000 single samples in a day, or more than 5,000 duplicate samples in a day.

In both the manual and UHT assays, antibody concentrations for each of the biomarkers are indicated in Table 2:

TABLE 2

| | | Detection Ab |
|---|---|---|
| Biomarker | Capture Ab Concentration | Concentration |
| CD5 | 0.285 μg/mL | 1 μg/mL |
| CD20 | 0.285 μg/mL | 2 μg/mL |
| Flt-3L | 0.285 μg/mL | 1 μg/mL |
| IL-15 | 0.285 μg/mL | 1 μg/mL |
| AMYIA | Immobilized: 0.285 pg/mL; | 10 μg/mL |
| | Non-immobilized: 2 | |
| | μg/mL | |

For quantitation of both the manual and UHT assays, eight calibrators and two control samples were run on each plate. Calibrators were prepared according to Table 3:

TABLE 3

| | Target Concentration (pg/mL) | | | | | | | |
|---|---|---|---|---|---|---|---|---|
| Analytes | Cal-1 | Cal-2 | Cal-3 | Cal-4 | Cal-5 | Cal-6 | Cal-7 | *Cal-8 |
| hu IL-15 | 2000 | 400 | 80 | 16 | 3.2 | 0.64 | 0.128 | 0 |
| hu CD5 | 6000 | 1200 | 240 | 48 | 9.6 | 1.92 | 0.384 | 0 |
| hu CD20 | 75,000 | 15000 | 3000 | 600 | 120 | 24 | 4.8 | 0 |
| hu Flt-3L | 7000 | 1400 | 280 | 56 | 11.2 | 2.24 | 0.448 | 0 |
| hu AMY1A | 2,000,000 | 400000 | 80000 | 16000 | 3200 | 640 | 128 | 0 |

Recovery of the control samples measured in 15 assay plates tested in 5 processing batches on 3 different days was assessed. The controls included negative and positive controls indicating high negative (i.e., <2 Gy) and low positive (i.e., >2 Gy) samples, prepared according to Table 4.

TABLE 4
Target Cone. (pg/mL)

| | External Negative | External Positive |
|---|---|---|
| Analytes | Control | Control |
| hu AMY1A | 120,000 | 230,000 |
| hu Flt3-Ligand | 468 | 1600 |
| hu CD20 | 1,000 | 60 |
| hu CD5 | 120 | 35 |
| hu IL-15 | 15 | 56 |

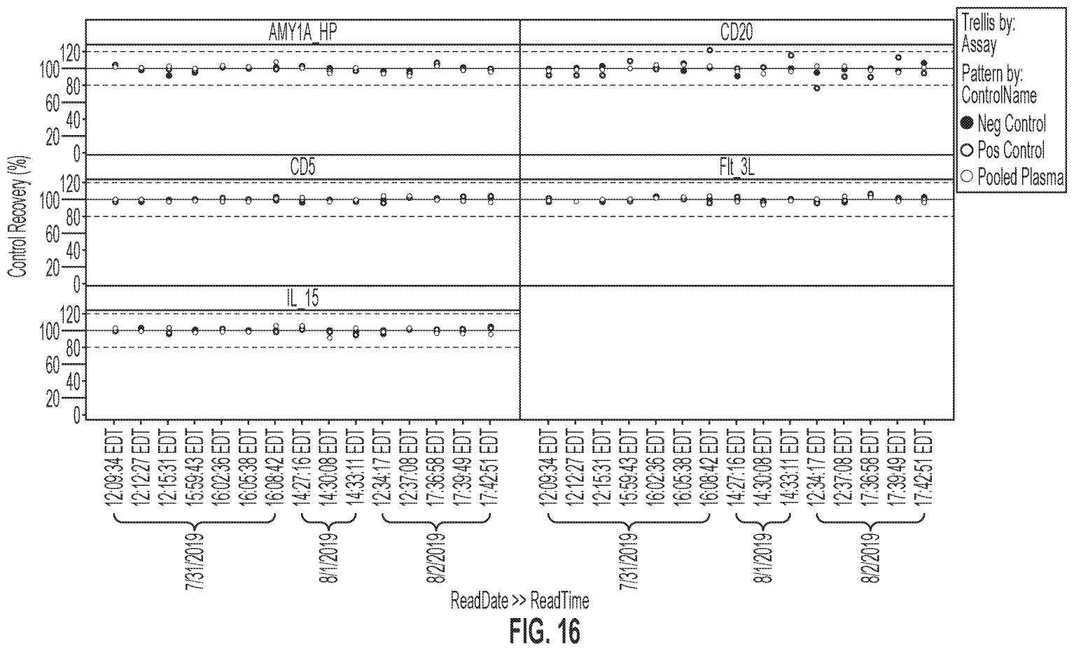

As shown in FIG. 16, the average coefficient of variation (CV) for intra-plate recovery is approximately 2.5%, and the average CV for inter-plate recovery is 3.4%.

### Example 9. Assay Formats

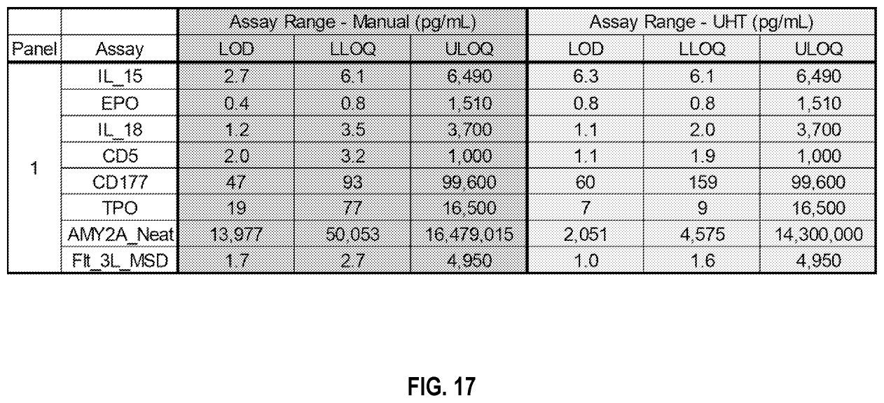

A multiplexed assay panel for IL-15, EPO, IL-18, CD5, CD177, TPO, AMY2A (desensitized), and Flt-3L was tested in both manual and ultra-high throughput (UHT) formats using an automated UHT instrument as described in Example 8. The manual assay format utilized liquid reagents while the UHT format utilized lyophilized reagents (i.e., detection reagent, calibrators, and external positive and negative controls) that were reconstituted in water prior to the assay. FIG. 17 shows a comparison of the assay parameters (LOD, LLOQ, and ULOQ) for the manual and UHT formats. LODs and LLOQs were lower for some biomarkers in the manual format, and lower for other biomarkers in the UHT format, but both assays generally had similar performance, indicating feasibility for using lyophilized reagents in an automated UHT instrument.

### Example 10. Multiplexed Assays

A multiplexed assay panel for AMY1A, CD5, CD20, Flt-3L, and IL-15 was tested in NHP and human plasma samples. NHP samples were obtained from individuals in a dose response study subjected to radiation doses of 0, 1, 2, 4, 6, and 8 Gy (TBI), and sampling time points of 0 (pre-irradiation), 1, 3, 5, and 7 days post irradiation. Human samples for a biomarker specificity study were obtained from 136 normal adult donors and 45 donors belonging to “special” populations based on age (adolescent or geriatric) or chronic diseases (chronic kidney disease, congestive heart failure, liver disease, or rheumatoid arthritis). Human stem cell transplant (SCT) patient samples were obtained from 8 individuals receiving SCT for blood cancers subjected to 12.5 to 13.25 Gy over 4 days in 1.25 Gy fractions.

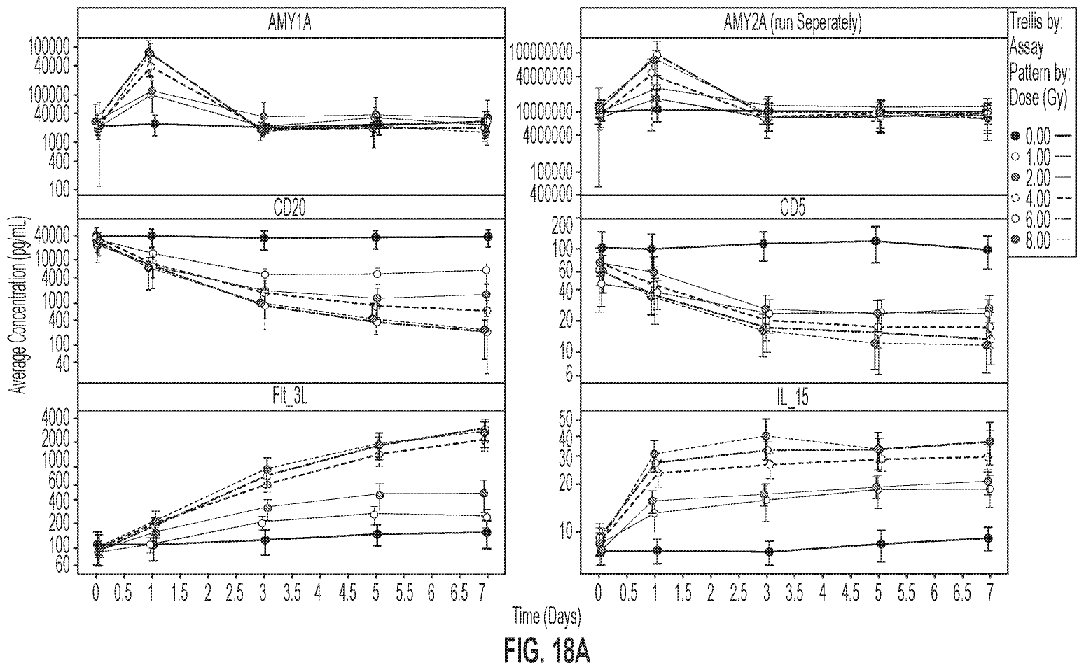

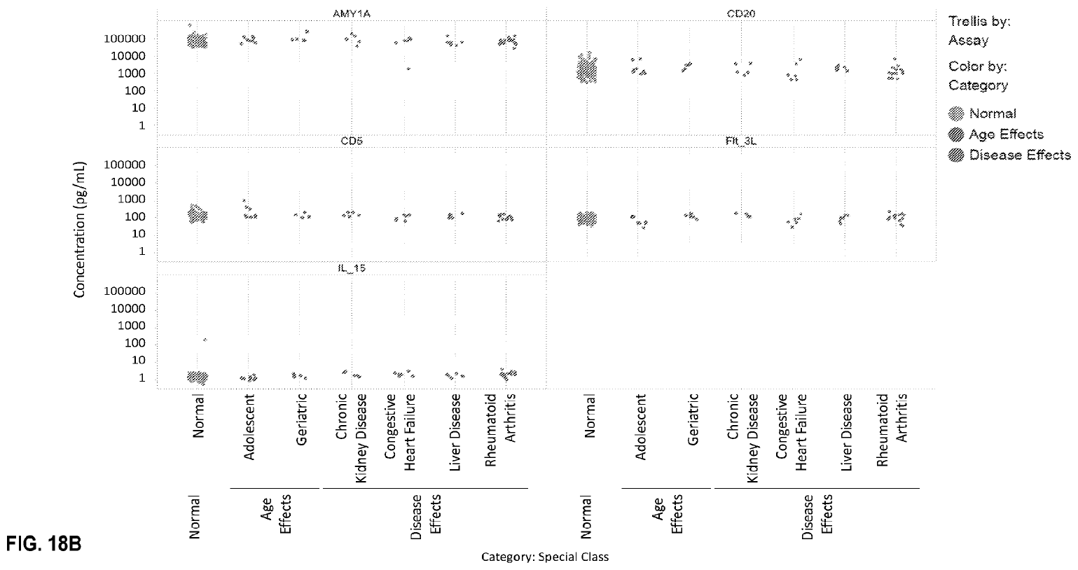

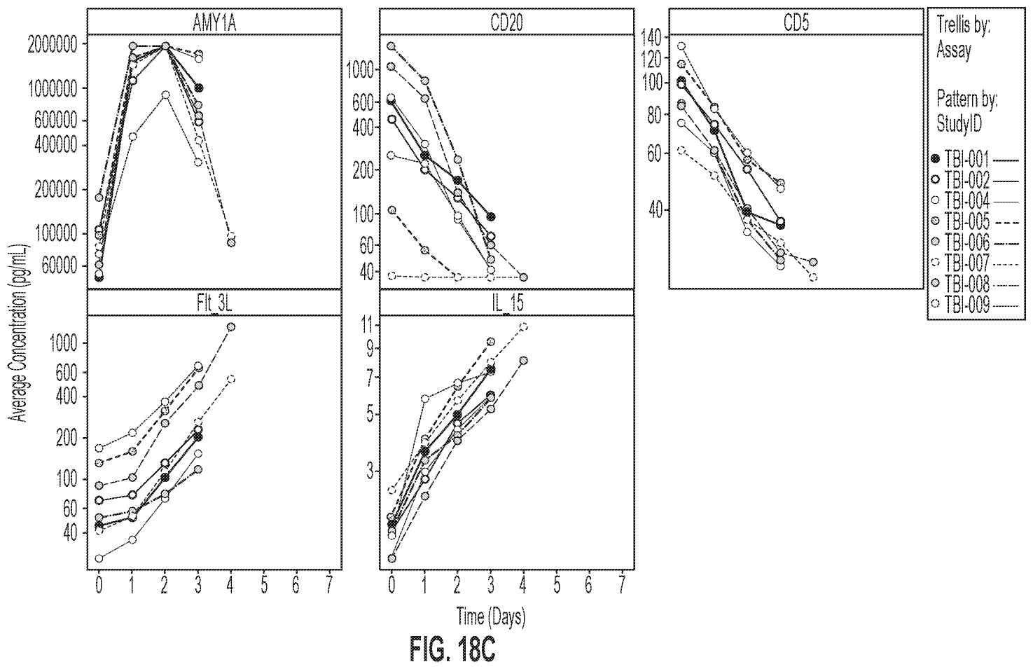

Results are shown in FIGS. 18A (NHP), 18B (human biomarker specificity study) and 18C (human SCT patient samples). NHP plasma samples were also tested separately for AMY2A, shown in FIG. 18A. The results in FIGS. 18A and 18C of NHP and humans subjected to radiation show that CD5 and CD20 decreased over time, while Flt-3L and IL-15 increased over time, as was observed in Examples 4 and 5. The Table in FIG. 28 shows the classification accuracy of a linear regression model for all negative (<3 Gy) and positive (≥3 Gy) samples. Column 3 of the Table in FIG. 28 shows the approximately 95% confidence interval range based on dose. As seen in FIG. 28, the regression model provided good specificity and sensitivity.

FIG. 18B shows a plot of biomarker levels for different subject categories, e.g., age or disease, indicating that the biomarkers included in the panel are not significantly different between normal and special populations. As discussed above, the Tables in FIGS. 24A and 24B show the observed specificities for the different classes of subjects.

### Example 11. Sample Matrix Component Interference Testing

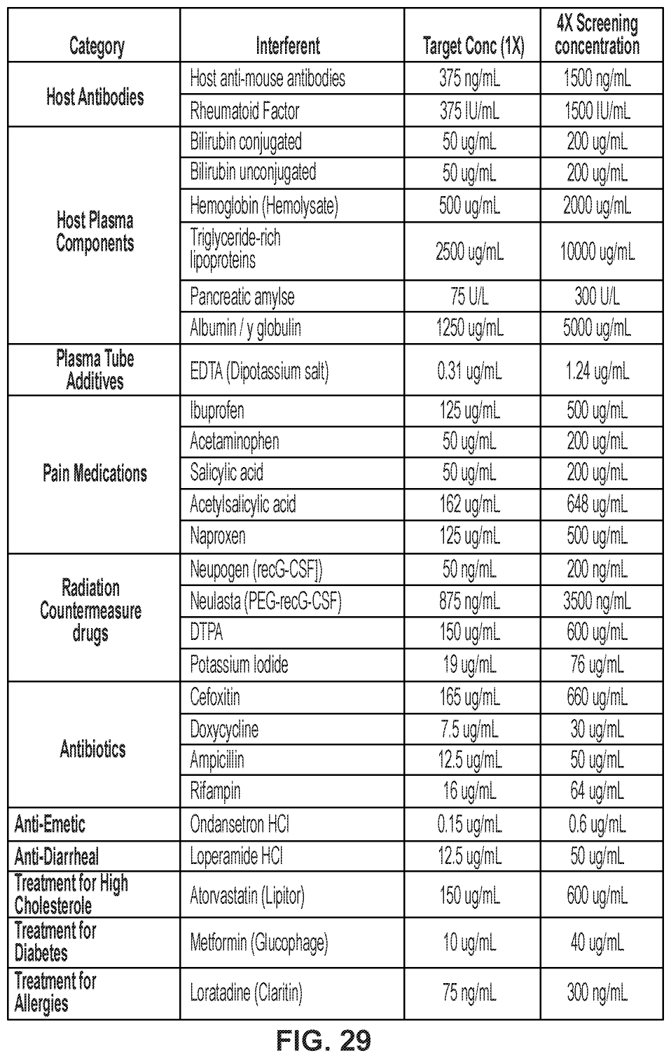

In this Example, components that are commonly present in sample matrices and can interfere with biomarker level measurements, also known as interferents, were tested for their ability to interfere with a biomarker panel assay. The list of interferents tested, organized by category, is shown in FIG. 29. The target concentration (1×) is the expected highest level of the interferent in a plasma sample.

The interferents were spiked into four different plasma samples at four times the target concentration, shown in FIG. 29 as the “4× Screening concentration.” The four plasma samples were then tested using the biomarker assay panel for measuring CD20, IL-15, AMY1A, CD5, and Flt-3L, as described in Example 7.

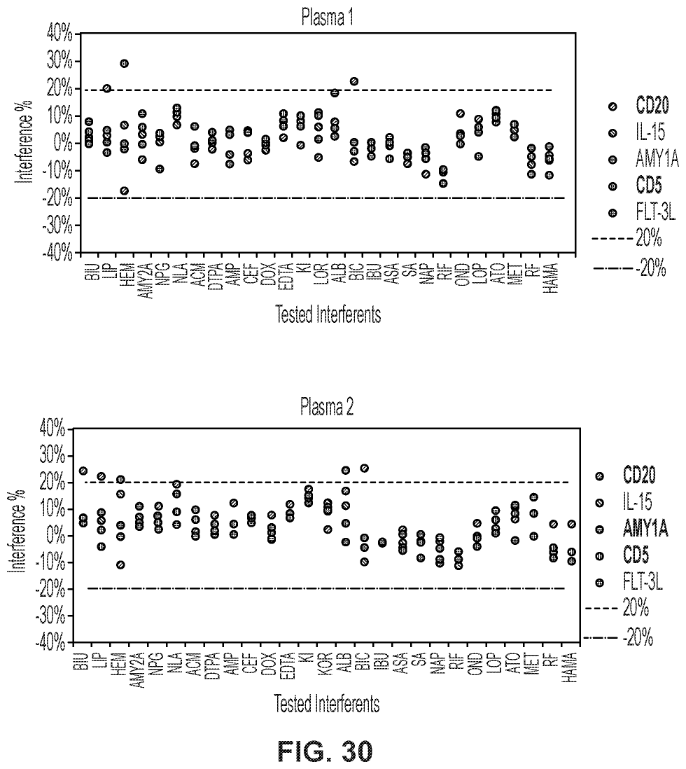

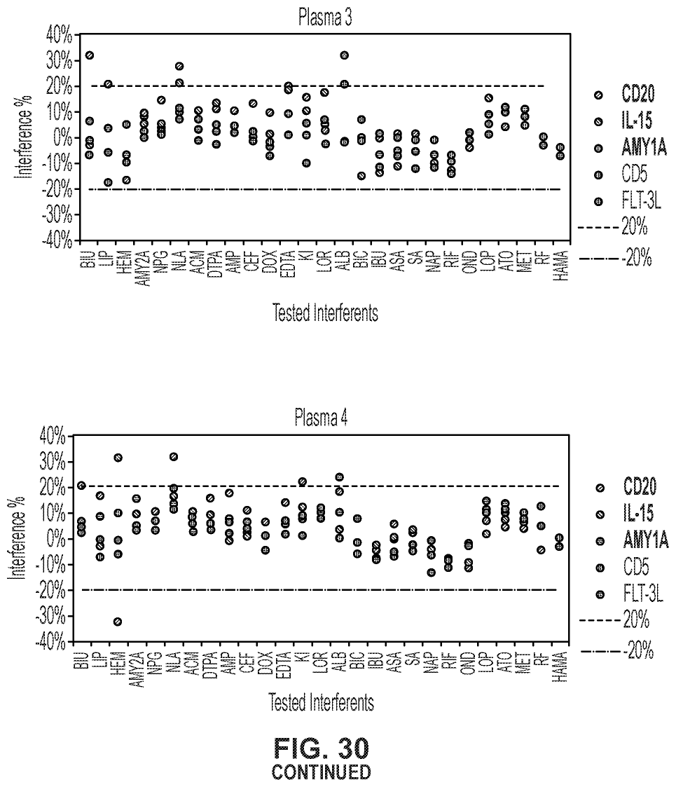

Results of the biomarker panel assays for the four plasma samples are shown in FIG. 30. The results indicate that for most of the interferents, their presence did not change the measured biomarker levels by more than 20%. The interferents that produced results outside of the 20% range for at least one biomarker were retested at lower concentrations.

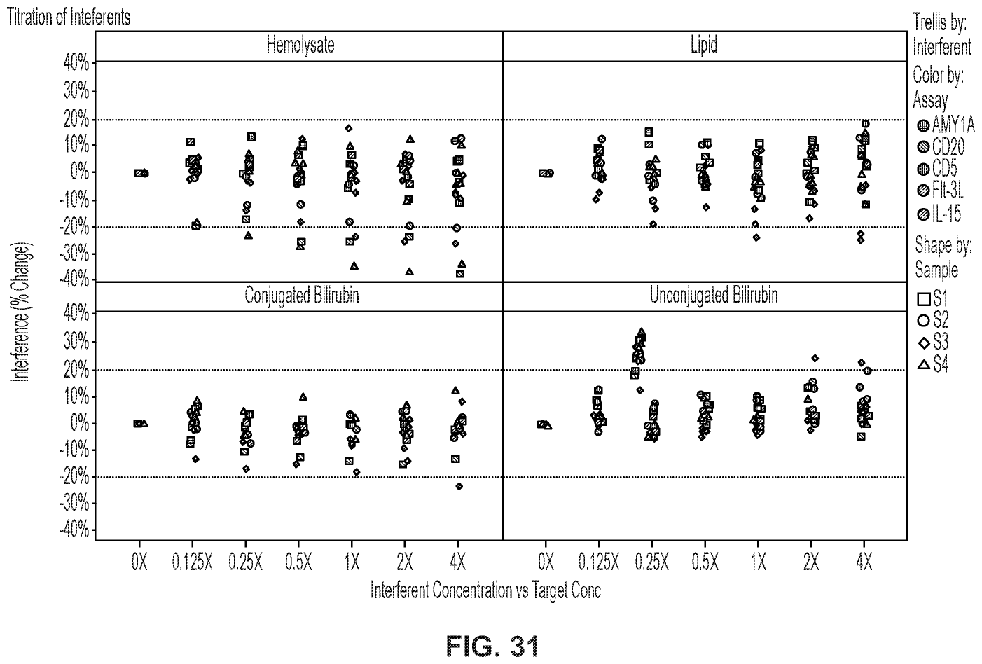

The experiment was repeated with four interferents, hemolysate, lipid, conjugated bilirubin, and unconjugated bilirubin, titrated into the four plasma samples at decreasing spike concentrations: 4×, 2×, 1×, 0.5×, 0.25×, 0.125×, and 0×, where 1× represents the expected highest level of the interferent in a plasma sample, as discussed above. The results are shown in FIG. 31. The results indicate that for lipid, conjugated bilirubin, and unconjugated bilirubin, interference was completely eliminated by the 1× target concentration titration. Hemolysate demonstrated some interference of the CD20 measurement at 1× target concentration, but the interference effect was reduced to acceptable levels by the 0.125× titration level. Thus, interference from hemolysate may be observed in samples with obvious red color from hemolysis.

### Example 12. Interference/Cross-Reactivity Testing

In this Example, the cross-reactivity and interference effect of each biomarker on the other biomarkers in a biomarker assay panel were tested. Normal plasma was individually spiked with each of the biomarkers in the biomarker assay panel for measuring CD20, IL-15, AMY1A, CD5, and Flt-3L, as described in Example 7. The biomarkers were spiked at levels two times higher than their highest expected level in normal or irradiated samples, as indicated in Table 5. Table 5 also shows the effects of the spiked biomarker on the measured concentrations of the other biomarkers, presented as a percent change in the measured concentration. Thus, the results showed that the highest expected level of one biomarker did not change the measured concentration of any other biomarker by more than 10%.

TABLE 5

| | Interference level | | | | |
|---|---|---|---|---|---|
| Spiked analyte | CD20 | IL-15 | AMYIA | CD5 | FLT-3L |
| Assay/Spike Level | 48 | 50 | 2 | 2.5 | 7 |
| | ng/mL | pg/mL | ug/mL | ng/mL | ng/mL |
| CD20 | NA | −0.2% | −1% | −2% | 2% |
| IL-15 | −3% | NA | 9% | 9% | 10% |
| AMY1A | 0% | −5% | NA | −4% | 1% |
| CD5 | −3% | −2% | −2% | NA | −2% |
| FLT-3L | −4% | −3% | −2% | −1% | N/A |

### Example 13. Stability of Assay Plates, Control, and Calibrators

The in-use stability of assay plates, containing the five-biomarker assay panel described in Example 7, was tested. The specified storage condition for the assay plate is storage in a desiccated foil pouch at 4° C. To test the plates' robustness to normal laboratory conditions, the plates were removed from the desiccated pouches and stored for up to 72 hours in the open air at either 25° C. (22 to 27° C.) or 37° C. (35 to 40° C.). The plates were then used to measure the concentrations of biomarkers in two control samples and three plasma samples. The measured concentrations were plotted after normalization to a plate that was stored at 4° C. and used immediately following removal from the foil pouch.

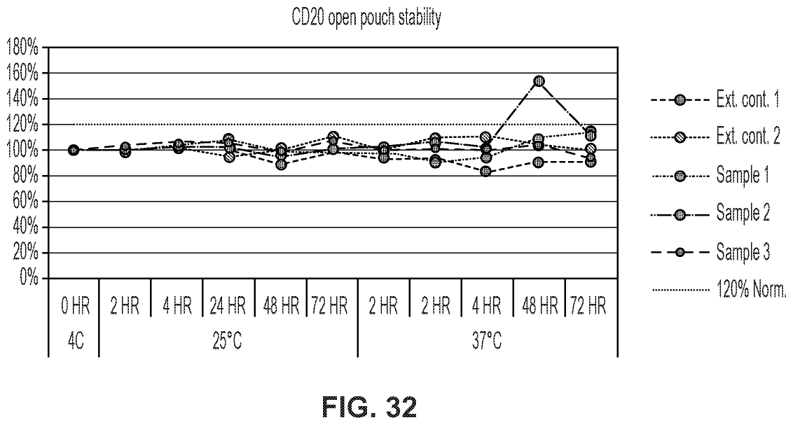

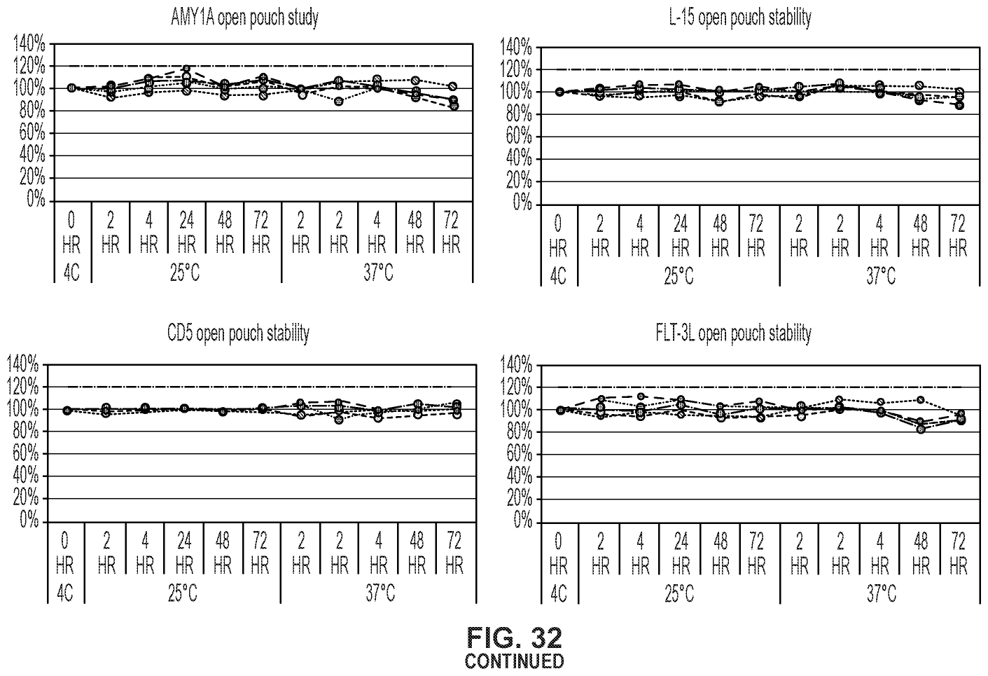

Results are shown in FIG. 32. The results indicate that under all storage conditions, the measured biomarker concentrations were within 20% of the concentration measured under the control condition. Based on this result, the assay plates were determined to be stable to air exposure for up to 72 hours at 37° C.

The in-use stability of control samples was also tested. To determine the robustness of lyophilized control samples to storage after reconstitution, the control samples were reconstituted and stored for up to 24 hours at 4° C. or 25° C. The control samples were then analyzed using the biomarker panel assay described in Example 7, and the measured concentrations were compared to the concentrations measured immediately after reconstitution.

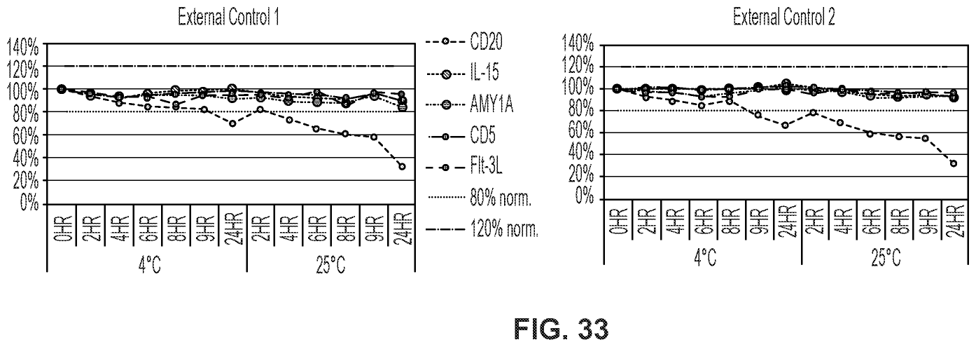

Results are shown in FIG. 33. The results indicated that the measured IL-15, AMY1A, CD5, and Flt-3L levels under all storage conditions were within 20% of the control condition, indicating that the levels of these four biomarkers in the control samples were stable to storage for up to 24 hours at 25° C. CD20 displayed instability, especially at the higher temperature condition. At 4° C., CD20 in the reconstituted control samples was stable for up to 8 hours.

The in-use stability of calibration standards (“calibrators”) was also tested. To determine the robustness of lyophilized calibrators to storage after reconstitution, the calibrators were reconstituted and stored for up to 24 hours at 4° C. or 25° C. The calibrators were then used in the biomarker panel assay described in Example 7 to determine the levels of the biomarkers in two control samples and three plasma samples. The measured concentrations of biomarkers in the control samples and plasma samples were compared to the concentrations that were measured using calibrators that were used immediately after reconstitution.

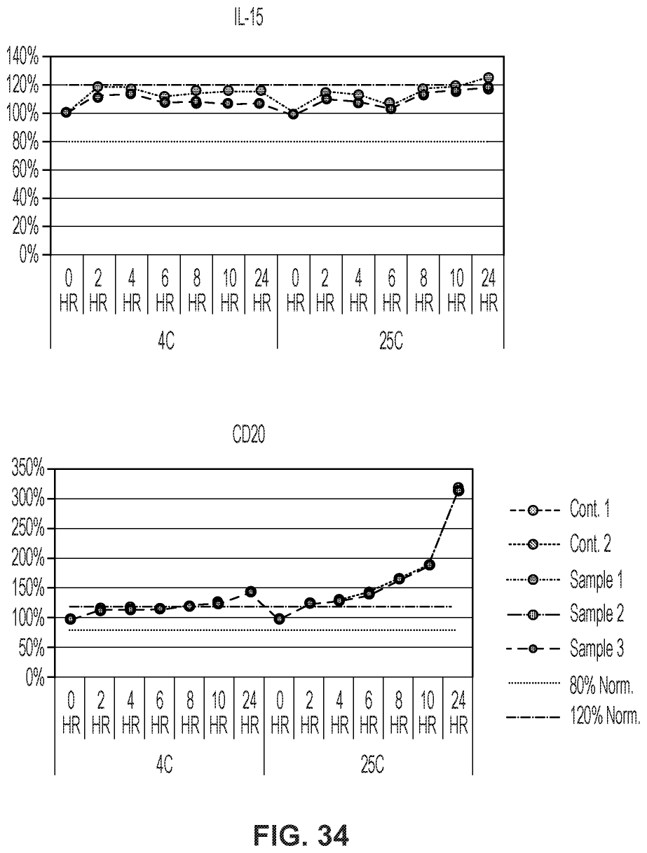

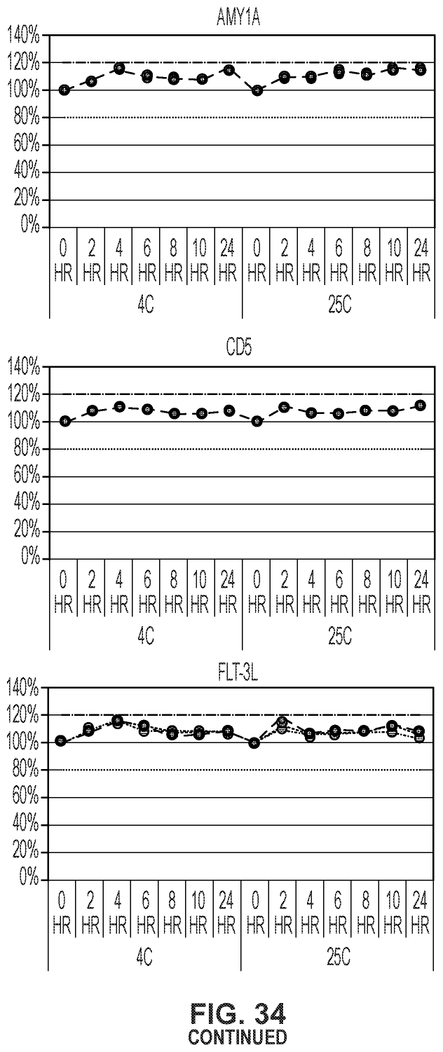

Results are shown in FIG. 34. The measured IL-15, AMY1A, CD5, and Flt-3L levels under all storage conditions were within 20% of the control condition, indicating that the levels of these four biomarkers in the calibration standards were stable to storage for up to 24 hours at 25° C. CD20 displayed instability, especially at the higher test temperature. At 4° C., CD20 in the reconstituted calibration standards was stable for up to 6 hours.

### Example 14. Stability of Plasma Samples

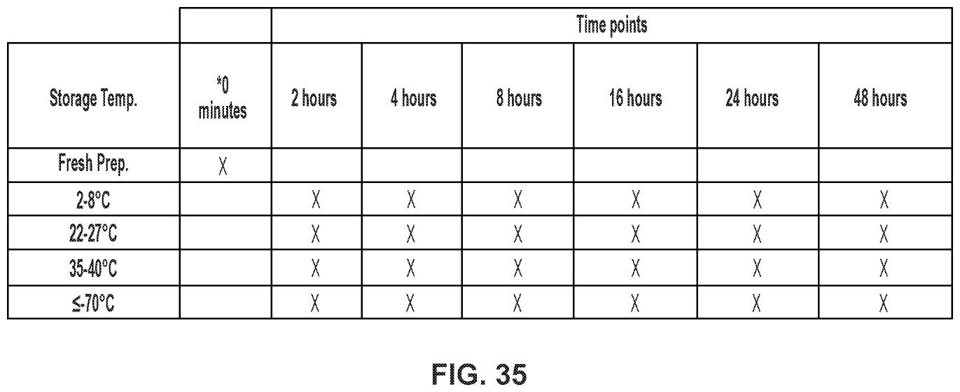

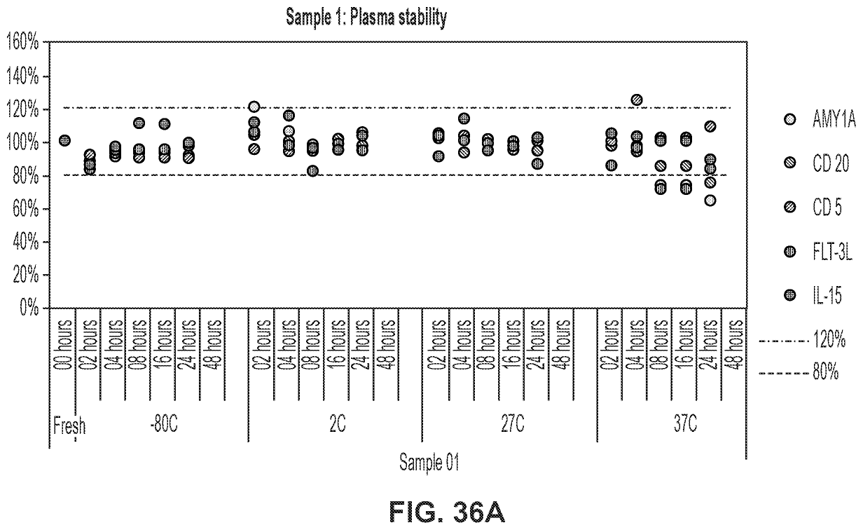

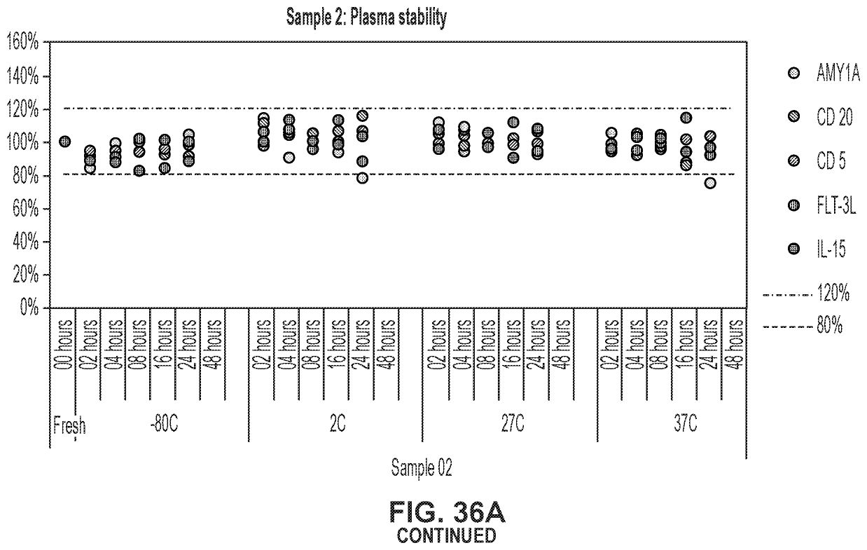

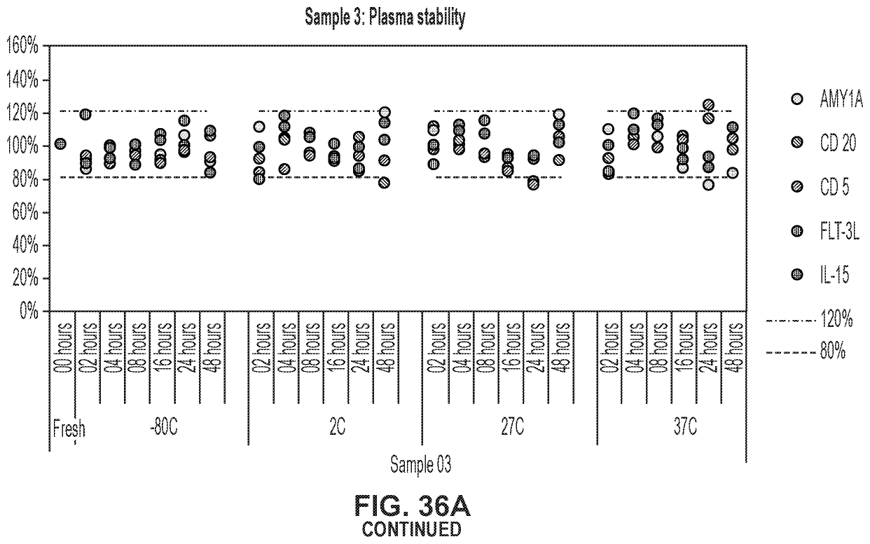

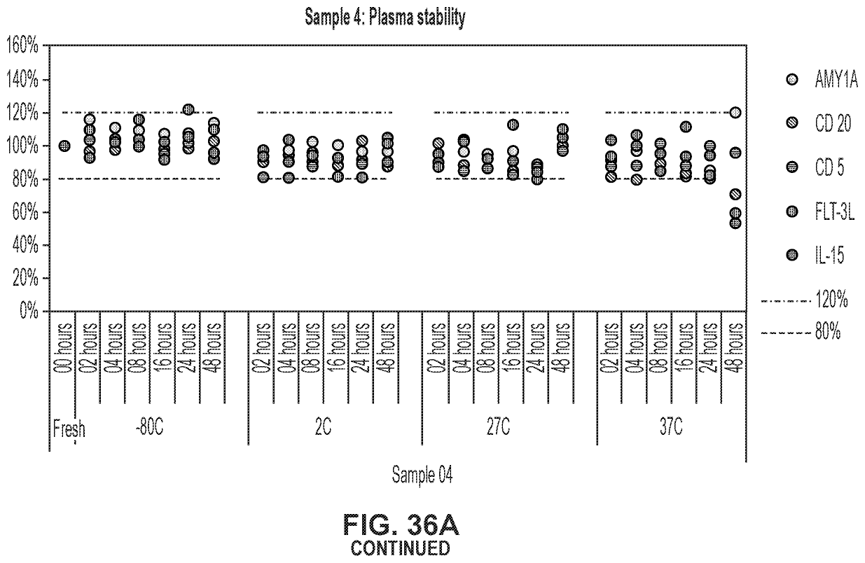

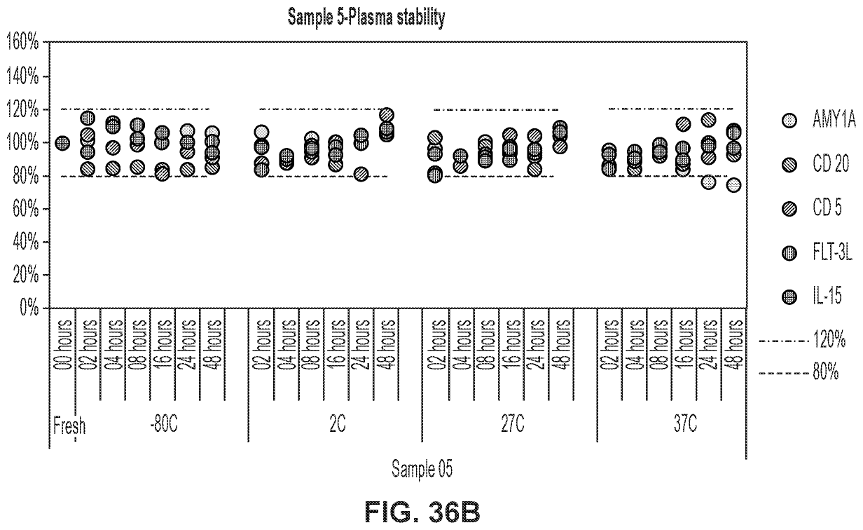

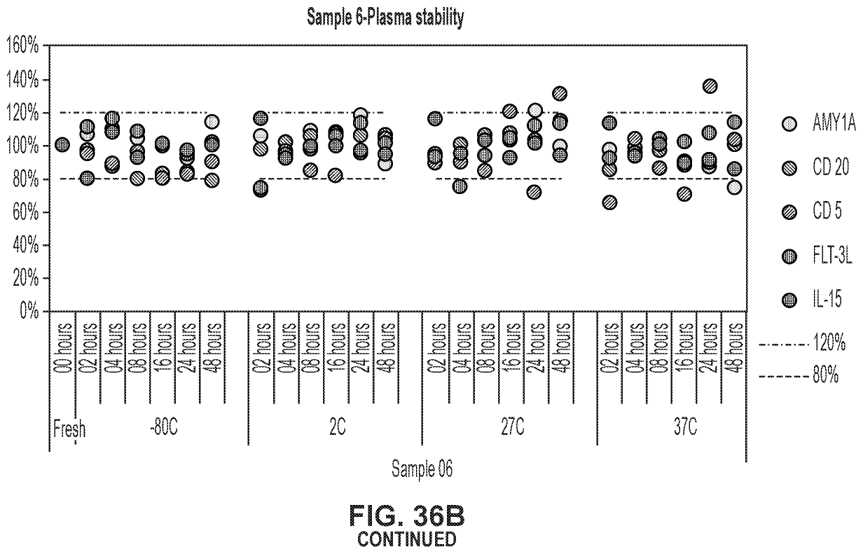

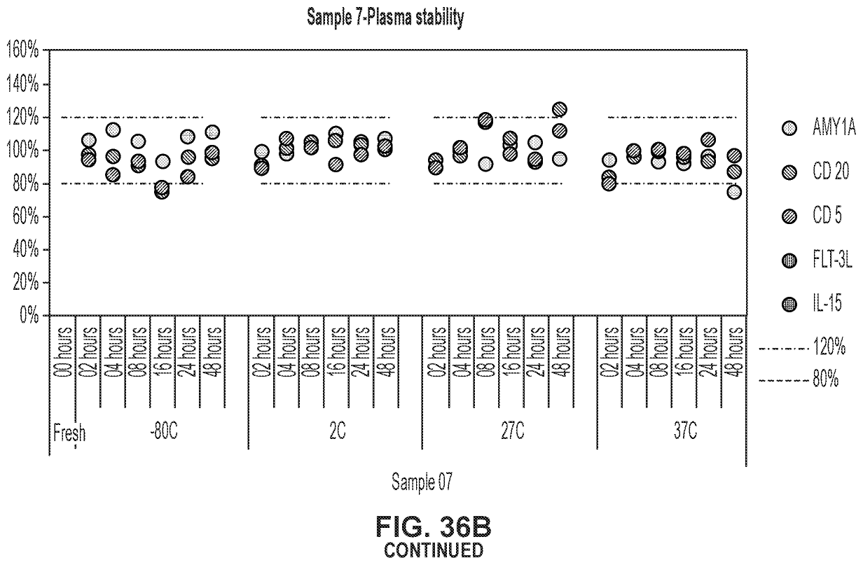

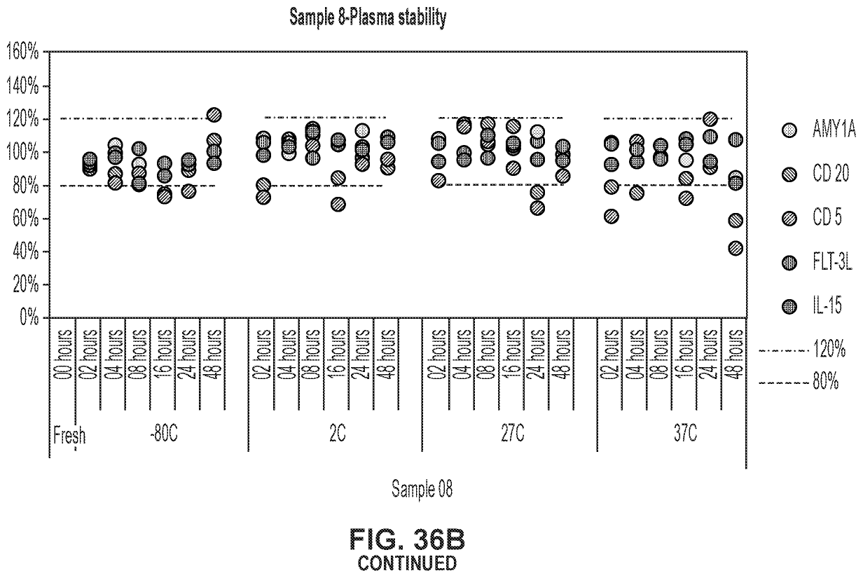

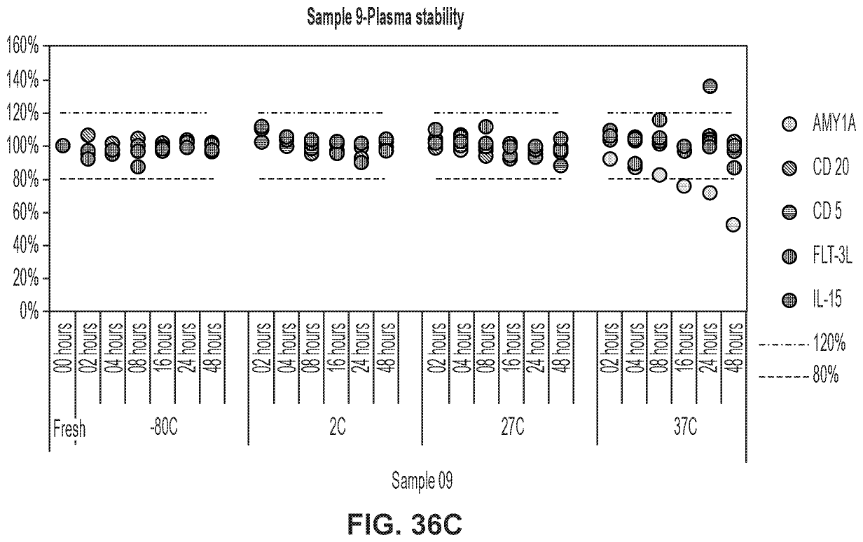

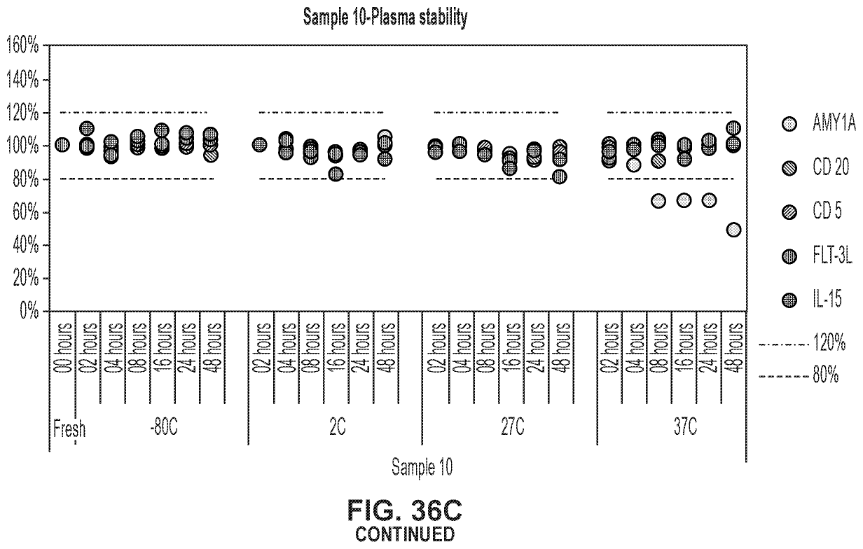

In this Example, the real time stability of plasma samples was tested. Fresh whole blood samples were obtained from ten individuals. Blood samples were processed within four hours of collection to produce plasma samples. Plasma samples were stored at different temperatures and for different amounts of time as described in FIG. 35. FIGS. 36A-36C show the measured concentration of each biomarker, as measured by the biomarker panel assay described in Example 7, for all ten samples at each time and temperature condition. The concentrations were normalized relative to a control aliquot that was stored at −80° C.

The results showed that the plasma samples were stable for all biomarkers at 23° C. for up to 48 hours, with measured biomarker levels within 80% of the control condition.

### Example 15. Algorithm Verification Testing with Nonhuman Primate (NHP) and Human Samples

Algorithm verification testing was performed with nonhuman primate (NHP) and human plasma samples. The cost function model (also called “error minimization (EM)” algorithm) and the linear regression model described herein were evaluated. The goal of the models is to identify samples from subjects that received doses above the critical dose (2 Gy for human, 3 Gy for NHP).

### Example 15.1. Control Experiment

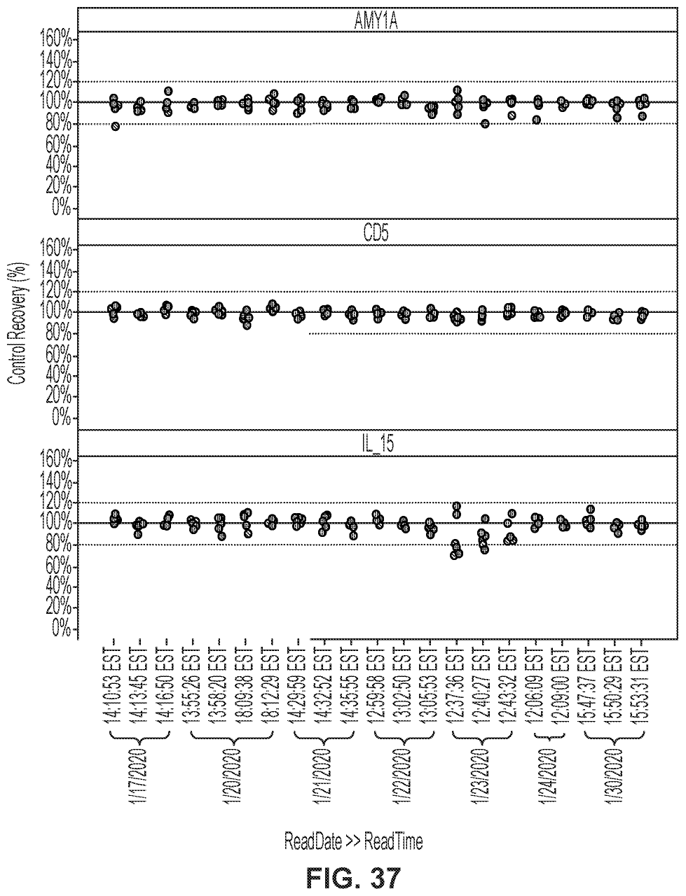

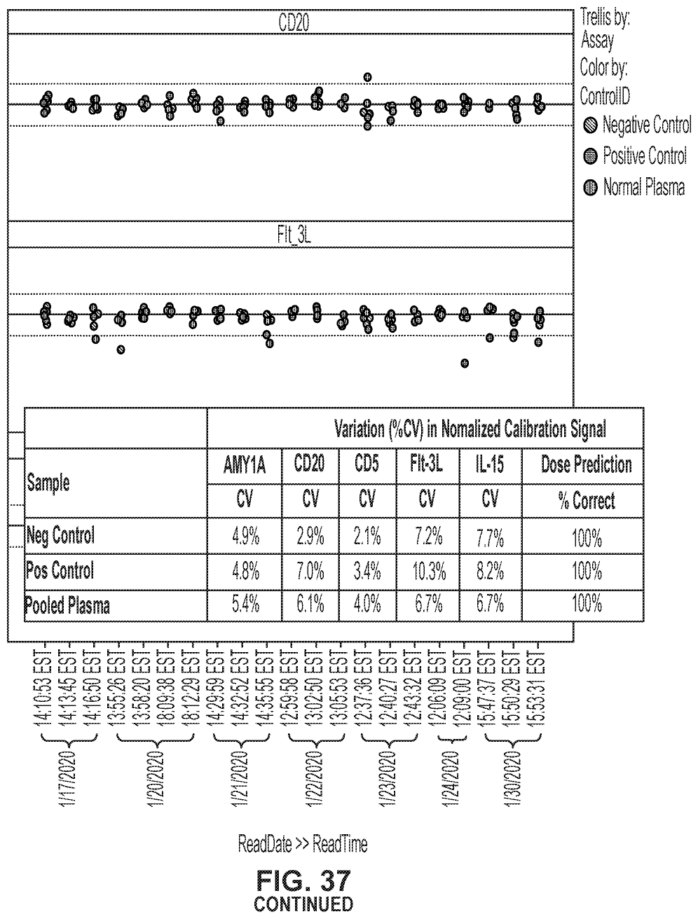

A control experiment was performed to with a positive control sample, a negative control sample, and a pooled plasma sample. FIG. 37 shows the results of measurements for the performed on 21 assay plates over the course of seven days with the samples in duplicate. The measured concentration is shown after normalization to the median value across the runs, and the inset table shows the measured coefficient of variations (CVs) for each control/assay combination across the experiment. The table also shows the percentage of the controls that provided the correct dose classification result (the negative and pooled plasma control should be classified as having a dose <2 Gy and the positive control should be classified as having a dose ≥2 Gy). As shown in FIG. 37, good reproducibility in control quantification was observed, with CVs ranging from about 2% to about 10%. All controls provided the expected dose classification.

### Example 15.2. NHP Radiation Dose Response Study

In the NHP (rhesus macaque) Dose Response Study, the NHP test subjects were subjected to 0, 2, 4, 6, 8, or 10 Gy, and plasma samples were collected before radiation (0 days) and 1, 3, 5, and 7 days after radiation. Ten animals were tested per dose condition. The algorithm training used an independent cohort (Cohort 2) from the same study. The doses for Cohort 2 were 0, 1, 2, 4, 6, and 8 Gy. The levels of AMY1A, CD20, CD5, Flt-3L, and IL-15 in the plasma samples were measured using a multiplexed assay. A separate assay was performed to measure the levels of NHP AMY2A, since the multiplexed assay panel measured for human AMY1A and did not quantitate amylase in all samples from normal NHP, as discussed in Example 10. The AMY2A values were used for dose prediction.

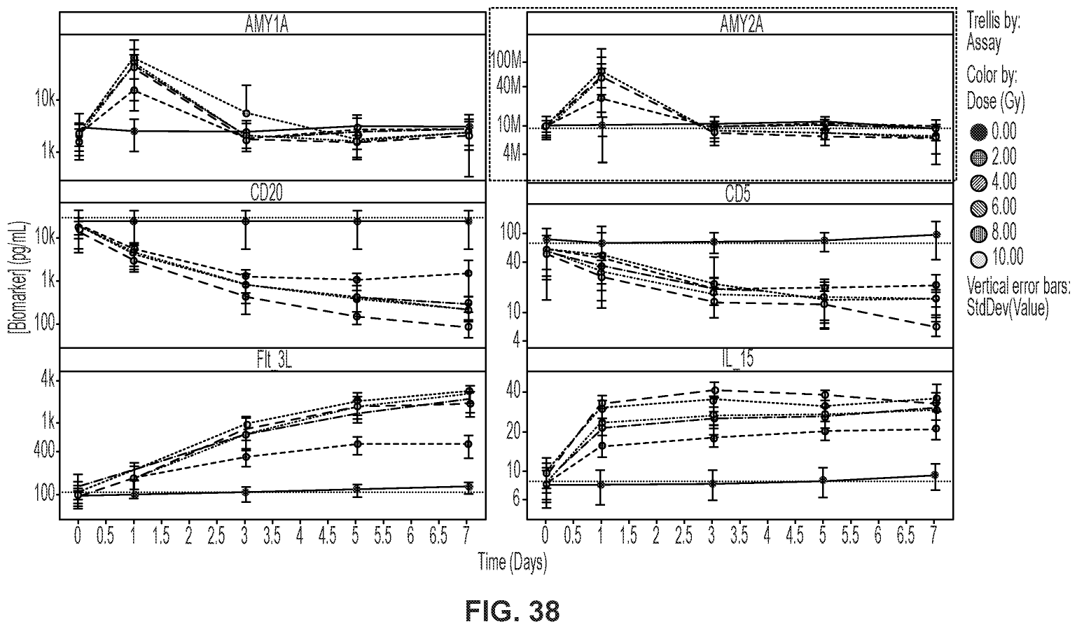

Results for the biomarker measurements are shown in FIG. 38. The biomarker level changes with the dose and time were as expected. The results for CD5, CD20, Flt-3L and IL-15 from the multiplexed panel and AMY2A from the NHP-specific AMY2A assay were used to assess accuracy of the dose assessment algorithms.

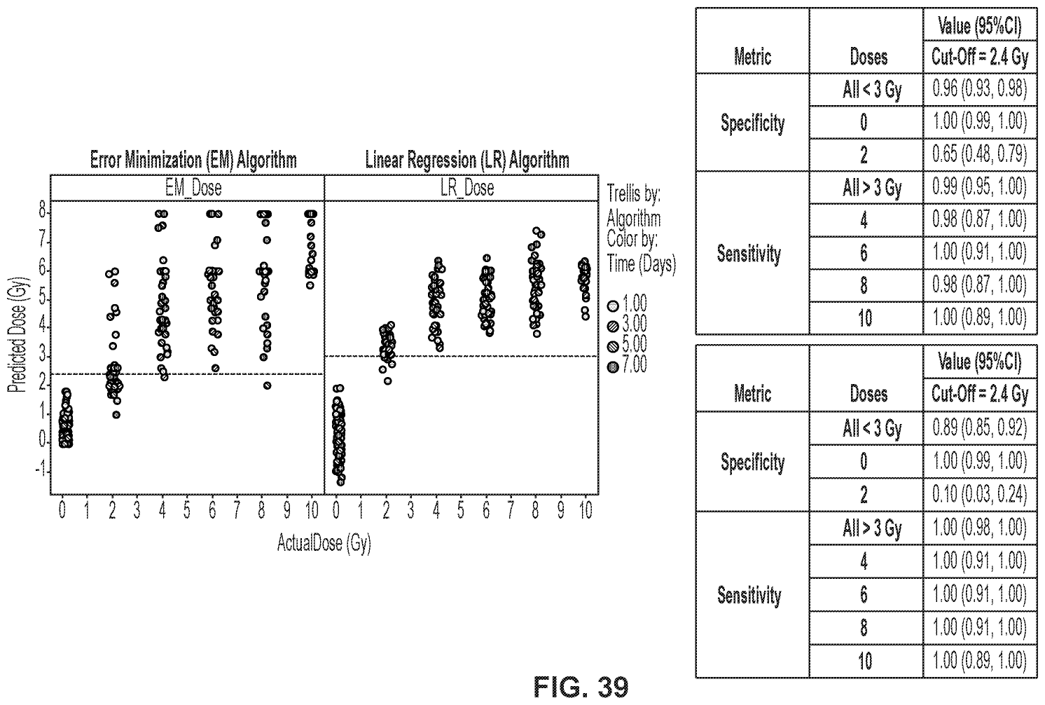

The performance accuracy of the dose assessment algorithms (cost function or error minimization and linear regression) is shown in FIG. 39, with the plots showing predicted dose as a function of actual dose with points colored based on time from exposure. The dashed vertical line represents the critical triage threshold level. The dashed horizontal line represents the cutoff predicted dose value used for classifying samples above or below the triage threshold. The tables provide the classification accuracy for all negative and positive samples, or stratified by dose (top: error minimization algorithm; bottom: linear regression algorithm). The 95% confidence intervals were estimated based on the binomial distribution. As demonstrated by these results, both algorithms provided good classification accuracy. The primary (cost function) algorithm provided better specificity at the high negative dose (2 Gy), while the alternative algorithm (linear regression) provided better sensitivity at the low positive dose (4 Gy). Similar results were observed during pre-verification.

### Example 15.3. NHP G-CSF Study

The NHP (rhesus macaque) G-CSF Study examined the effects of administration of a radiation countermeasure, G-CSF, on dose prediction. In the NHP (rhesus macaque) G-CSF Study, the NHP test subjects were subjected to 0 or 6 Gy. For each of the two doses, there were two treatment arms: 0 or 10 μg/kg G-CSF daily, starting at day 1 post-exposure. Plasma samples were collected before radiation (0 days), and a number of time points after radiation, from 1 to 33 days. The levels of AMY1A, CD20, CD5, Flt-3L, and IL-15 in the plasma samples were measured using a multiplexed assay. A separate assay was performed to measure the levels of NHP AMY2A, as discussed in Example 15.2.

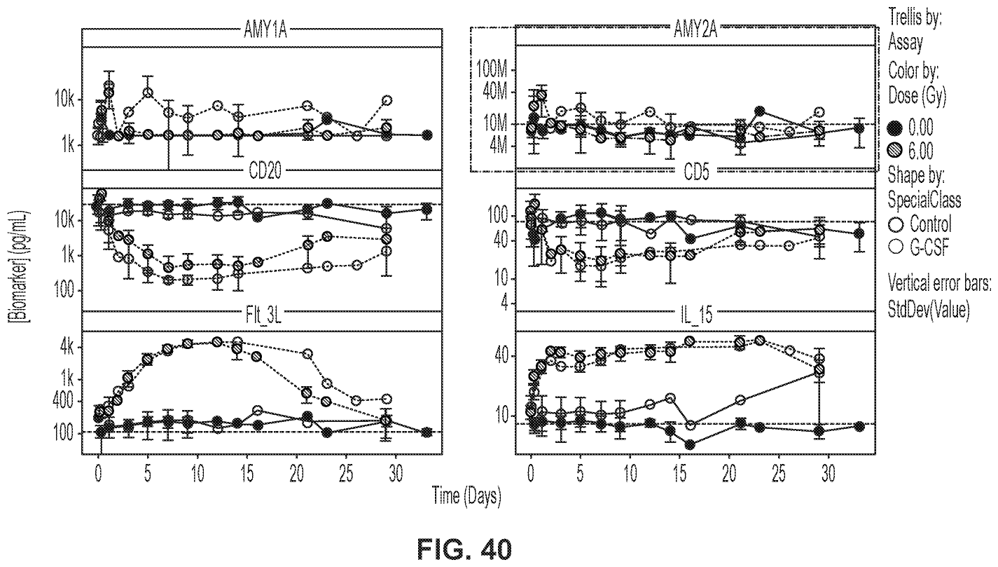

Results for the biomarker measurements are shown in FIG. 40. Each data point represents the average level measured across samples from the four animals in the G-CSF arm) or the two animals in the control treatment arm. As shown in FIG. 40, administration of G-CSF did not have a significant effect on biomarker levels for non-irradiated animals. G-CSF administration reduced the rate at which CD20 levels dropped after radiation exposure and provided a faster recovery in the levels of both CD20 and Flt-3L. One animal receiving radiation, but in the control treatment arm, showed unexpected elevations in AMY levels for time points one month post radiation and later. The results at longer time points showed that biomarker changes observed at 7 days post radiation presist for at least another week, regardless of G-CSF administration.

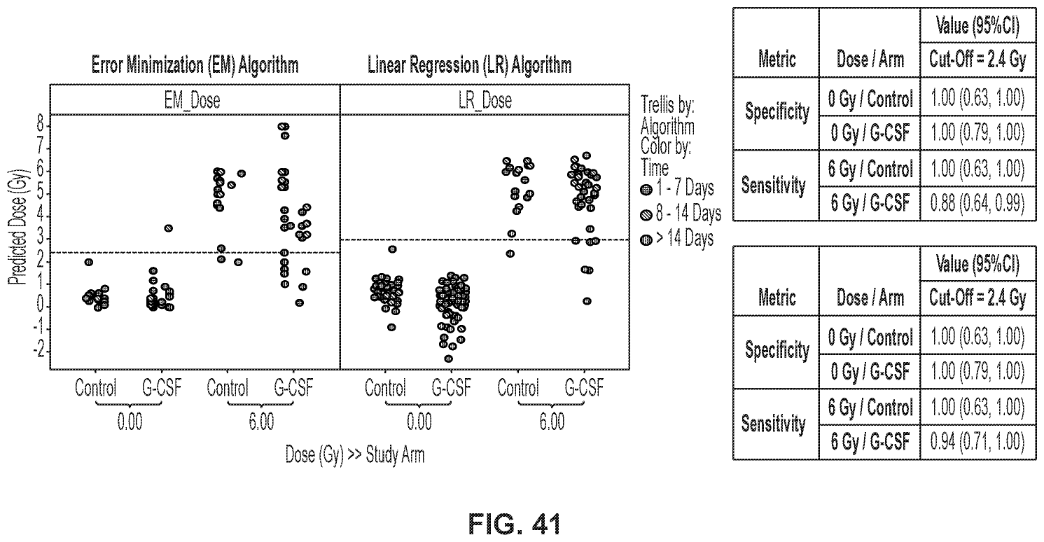

Performance accuracy of the dose assessment algorithms (cost function or error minimization and linear regression) are shown in FIG. 41, with the plots showing predicted dose as a function of actual dose and whether the study animals received G-CSF after irradiation. The points are colored based on time from exposure. The dashed horizontal line is the cutoff predicted dose value used for classifying samples as above or below the triage threshold. The tables provide the classification accuracy for all negative and positive samples, stratified by drug treatment arm (top: error minimization algorithm; bottom: linear regression algorithm). The 95% confidence intervals were estimated based on the binomial distribution. As demonstrated by these results, both algorithms provided good specificity regardless of whether the animals received G-CSF. Some evidence of reduced sensitivity was observed for irradiated animals in the G-CSF arm, especially for the cost function algorithm, however, false negatives were only observed for one animal.

### Example 15.4. Biomarker Levels in Human Plasma Samples from Normal and Special Populations

In this Study, dose classification specificity and robustness to potential confounding factors based on age, injury, disease, or special conditions were evaluated. Biomarker levels were determined in archived human plasma samples from 100 normal adult donors and samples belonging to “special” populations based on (1) age: 8 adolescent (ages 18-21) samples and 5 geriatric (ages 62-72) samples; (2) injury: 5 samples from burn patients (5-15% second or third degree burns) and 5 samples from wound patients (3 penetrating wounds, 2 non-penetrating wounds); (3) disease or special condition: 16 samples from anemia patients, 9 samples from asthma patients, 5 samples from chronic kidney disease, 6 samples from congestive heart failure patients, 9 samples from chronic obstructive pulmonary disease (COPD) patients, 6 samples from diabetes patients, 6 samples from hypertension patients, 5 samples from inflammatory bowel disease patients, 6 samples from liver disease patients, 3 samples from lupus patients, 10 samples from pregnant women, 14 samples from rheumatoid arthritis patients, and 4 samples from sepsis patients. The levels of AMY1A, CD20, CD5, Flt-3L, and IL-15 in the plasma samples were measured using a multiplexed assay.

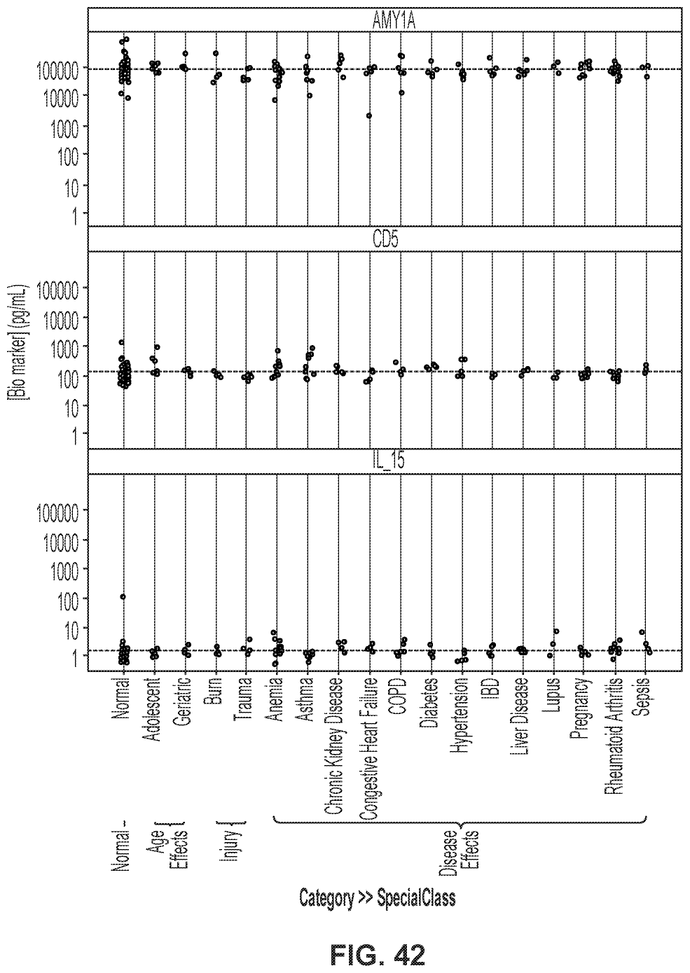

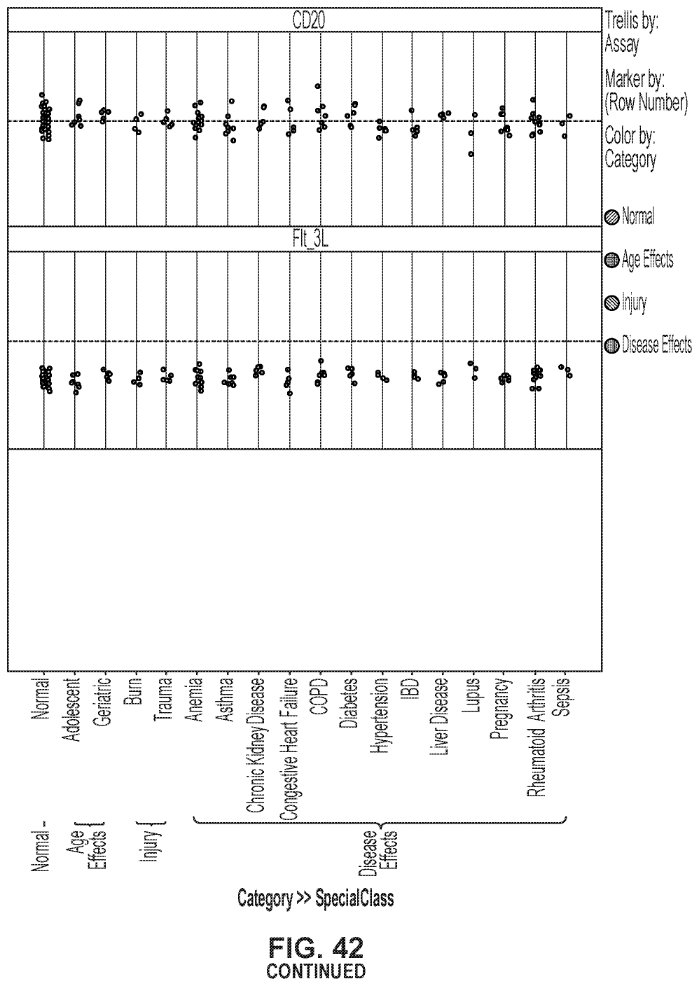

Results for the biomarker measurements are shown in FIG. 42. The horizontal line in each panel represents the median normal biomarker level as determined during pre-verification studies. In general, large differences in biomarker levels were not observed in the special population samples relative to the normal adult samples. The measured biomarker values were used to test the specificity of the dose assessment algorithms.

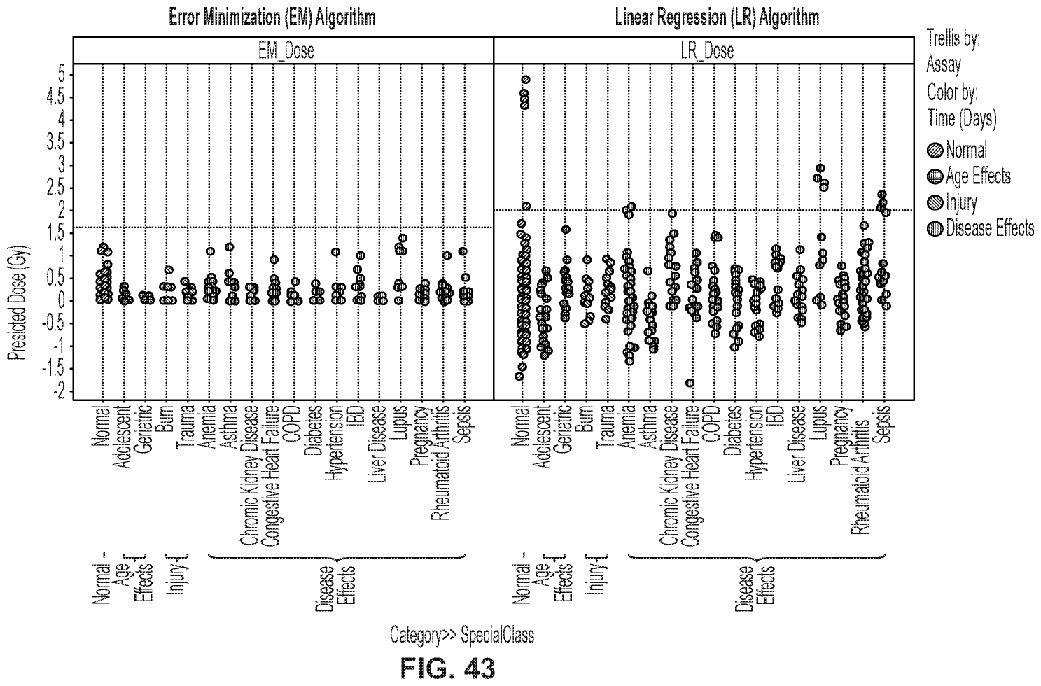

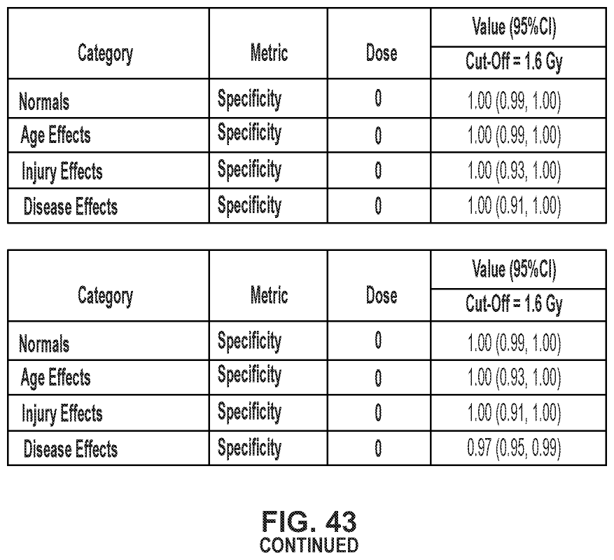

Specificity of dose assessment algorithms (cost function or error minimization and linear regression) are shown in FIG. 43. The tables show the observed specificities for the different classes of subjects (top: error minimization algorithm; bottom: linear regression algorithm). The 95% confidence intervals were estimated based on the binomial distribution. As demonstrated by these results, excellent specificity was observed using both algorithms. No false positives were observed for the normal samples and samples from special age groups or injured subjects. The small number of false positives that were observed were with the linear regression algorithm and mostly fell within the lupus and sepsis groups, possibly because of slightly elevated IL-15 levels in these groups.

### Example 15.5. Biomarker Levels in Human Plasma Samples from Stem Cell Transplant (SCT) Patients

In this Study, dose classification algorithms were evaluated for human patients receiving total body irradiation (TBI). Biomarker levels were determined in archived human plasma samples from cancer (AML, ALL) patients receiving fractionated TBI prior to stem cell transplant. The total dose varied from 9.6 Gy to 13.2 Gy, with 1.2 to 1.5 Gy per fraction and the fractions delivered over 4 days. Sampling was performed on Day 0 (pre-TBI), 1, 2, 3, and 4. The cohort size was 12 SCT patients. The levels of AMY1A, CD20, CD5, Flt-3L, and IL-15 in the plasma samples were measured using a multiplexed assay.

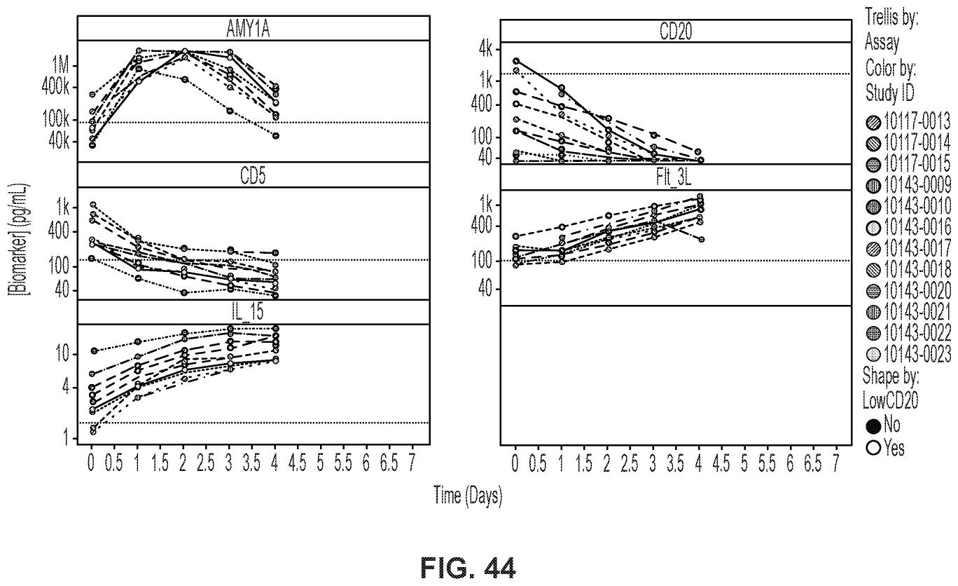

Result for the biomarker measurements are shown in FIG. 44. As expected from previous testing of SCT patients, several patients had atypically low baseline CD20 levels, which may be due to the loss of CD20 cells in previous rounds of chemotherapy. Patients with undetectable baseline CD20 (<37 pg/mL) are indicated with open circles and were excluded in the verification testing. The measured biomarker values were used to evaluate performance of the dose prediction algorithms.

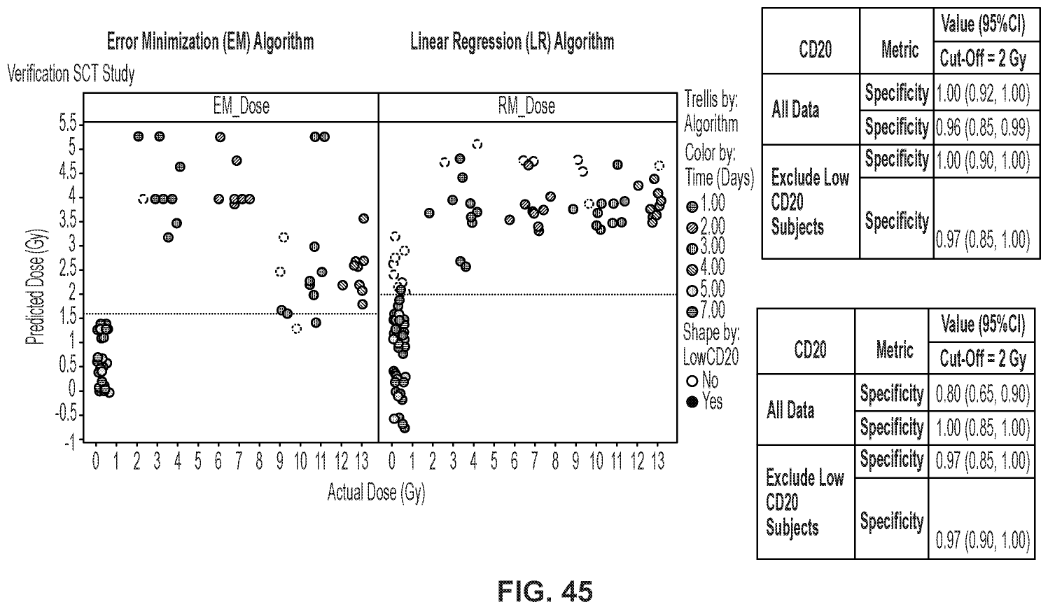

Performance of dose assessment algorithms (cost function or error minimization and linear regression) are shown in FIG. 45 as the dose prediction for SCT patient samples as a function of total dose. Points are color coded based on time from first fraction. Plots are provided for the primary (cost function or error minimization) and alternative (linear regression) algorithms. Results from the two subjects with undetectable CD20 at baseline are shown as open circles. The tables show specificity and sensitivity for the full data set, and after removing data from subjects with undetectable CD20 at baseline (top: error minimization algorithm; bottom: linear regression algorithm). As demonstrated by these results, both algorithms performed well, especially after removing subjects with undetectable CD20 at baseline. The specificity was better for the cost function algorithm than the linear regression algorithm when all subjects were included. This may not be a result of the lower baseline CD20 levels for these subjects, but because these subjects also have higher than normal IL-15 levels.

What is claimed is:

1. A multiplexed immunoassay method comprising, quantifying the amounts of at least four human biomarkers in a biological sample, wherein the at least four biomarkers comprise (a) IL-15, (b) CD5, (c) Flt-3L, and (d) salivary amylase, wherein the quantifying comprises measuring the concentrations of the at least four biomarkers in a multiplexed assay format to simultaneously measure the concentrations of the least four biomarkers in the biological sample wherein the multiplexed immunoassay comprises:
a. combining, in one or more steps:
i. the biological sample;
ii. at least a first, second, third, and fourth binding reagent, wherein the first, second, third, and fourth binding reagent is a binding partner of IL-15, CD5, Flt-3L, and salivary amylase, respectively;
b. forming at least a first, second, third, and fourth binding complex comprising the binding reagents and the biomarkers;
c. measuring the concentration of the biomarkers in each of the binding complexes.

2. The method of claim 1, wherein the first, second, third and fourth binding reagents are immobilized on associated first, second, third and fourth binding domains, and the measuring step comprises measuring the complexes in each of the binding domains.

3. The method of claim 2, further comprising combining the biological sample with a non-immobilized competing reagent that competes with the first, second, third or fourth binding reagent for binding to its target biomarker, and desensitizes the measurement of that biomarker.

4. The method of claim 3, wherein the non-immobilized competing reagent competes with the fourth binding reagent for binding to salivary amylase.

5. The method of any of claims 1 to 4, wherein the components combined in step (a) further comprise at least a first, second, third, and fourth detection reagent that each bind a biomarker, and the binding complexes formed in step (b) further comprise the at least first, second, third, and fourth detection reagents.

6. The method of claim 5, wherein the detection reagents each comprises a detectable label.

7. The method of claim 5 or 6, wherein the binding reagents and the detection reagents are antibodies.

8. The method of any of claims 5 to 7, wherein the measuring the concentration comprises measuring the presence of the detectable labels by electrochemiluminescence.

9. The method of any of claims 1 to 8, wherein each of the binding domains is an element of an array of binding domains.

10. The method of claim 9, wherein the array is located within a well of a multi-well plate.

11. The method of any of claims 1 to 9, wherein each of the binding domains are positioned on a surface of one or more particles.

12. The method of any of claims 6 to 11, wherein the detectable label is an electrochemiluminescence label, and the measuring of the detectable label comprises measuring an ECL signal.

13. The method of any of claims 1 to 12, wherein the biological sample is whole blood, serum, plasma, cerebrospinal fluid, urine, saliva, or an extraction or purification therefrom, or dilution thereof.

14. The method of claim 13, wherein the biological sample is serum or plasma.

15. The method of any of claims 1 to 14, further comprises measuring, in the multiplexed assay format, at least one additional biomarker in the biological sample, wherein the at least one additional biomarker is CD20, IL-18, CD27, thyroid peroxidase (TPO), or a combination thereof.

16. The method of any of claims 1 to 15, wherein the biological sample is obtained from a subject exposed to radiation, at risk of exposure to radiation or suspected of having been exposed to radiation.

17. A multiplexed immunoassay method comprising, quantifying the amounts of at least four human biomarkers in a biological sample, wherein the at least four biomarkers comprise IL-15, CD5, Flt-3L, and salivary amylase, wherein the quantifying comprises measuring the concentrations of the at least four biomarkers in a multiplexed assay format to simultaneously measure the concentrations of at least four biomarkers in the biological sample, wherein the multiplexed immunoassay comprises:
a. combining, in one or more steps:
i. the biological sample;
ii. at least a first antibody to IL-15; a first antibody to CD5; a first antibody to Flt-3L; and a first antibody to salivary amylase, wherein each of the first antibodies is immobilized on separate binding domains;
iii. at least a second antibody to IL-15; a second antibody to CD5; a second antibody to Flt-3L; and a second antibody to salivary amylase, wherein each second antibody is connected to a detectable label;
b. forming at least a first, second, third, and fourth binding complex on an at least first, second, third, and fourth binding domains comprising at least IL-15, CD5, Flt-3L, and salivary amylase, and the first and second antibodies for their respective biomarker;
c. measuring the concentration of the at least IL-15, CD5, Flt-3L, and salivary amylase on the at least first, second, third, and fourth binding domains.

18. The method of claim 17, further comprising combining the biological sample with a non-immobilized competing reagent that competes with a first or second antibody to one of the at least four biomarkers for binding to its target biomarker, and desensitizes the measurement of that biomarker.

19. The method of claim 18, wherein the non-immobilized competing reagent competes with the first antibody for binding to salivary amylase.

20. The method of any of claims 17 to 19, further comprising measuring, in the multiplexed assay format, at least one additional biomarker in the biological sample, wherein the at least one additional biomarker is CD20, IL-18, CD27, thyroid peroxidase (TPO), or combination thereof.

21. The method of claim 20, wherein the at least one additional biomarker is CD20, IL-18, or both.

22. A method of determining radiation exposure in a human, comprising
a. conducting the multiplexed immunoassay of any of claims 1 to 21 on a biological sample of a human,
b. detecting the concentration of biomarker IL-15, biomarker CD5, biomarker Flt-3L, and biomarker salivary amylase,
c. determining if:
i. the concentration of biomarker IL-15 is higher compared to a control;
ii. the concentration of biomarker CD5 is lower compared to a control;
iii. if the concentration of biomarker Flt-3L is higher compared to a control;
iv. if the concentration of salivary amylase is higher or the same compared to a control,
wherein if any of (i), (ii), (iii) or (iv) is true, reporting that the human has been exposed to radiation, wherein the control of (i), (ii), (iii), and (iv) is from a human who has not been exposed to radiation.

23. The method of claim 22, wherein (b) further comprises detecting the concentration of biomarker CD20, biomarker IL-18, biomarker CD27, biomarker TPO, or combination thereof, and wherein (c) further comprises determining:
v. if the concentration of biomarker CD20 is lower compared to a control;
vi. if the concentration of biomarker IL-18 is higher compared to a control;
vii. if the concentration of biomarker CD27 is lower compared to a control;
viii. if the concentration of biomarker thyroid peroxidase (TPO) is higher compared to a control;
wherein if any of (i) to (viii) is true, reporting that the human has been exposed to radiation, wherein the control of (i) to (viii) is from a human who has not been exposed to radiation.

24. The method of claim 22 or 23, further comprising administering an agent for treating radiation exposure in a human.

25. A kit comprising, in one or more vials, containers, or compartments:
(a) a surface comprising at least a first, second, third, and fourth binding reagent immobilized on an associated first, second, third, and fourth binding domain, wherein the first, second, third, and fourth binding reagent is a binding partner of IL-15, CD5, Flt-3L, and salivary amylase, respectively;
(b) a detection reagent that specifically binds to biomarker IL-15;
(c) a detection reagent that specifically binds to CD5;
(d) a detection reagent that specifically binds to Flt-3L; and
(e) a detection reagent that specifically binds to salivary amylase.

26. The kit of claim 25, further comprising a calibration reagent, a control reagent, or both.

27. The kit of claim 25 or 26, wherein each binding reagent and detection reagent is an antibody.

28. The kit of any of claims 25 to 27, wherein each detection reagent comprises a detectable label.

29. The kit of any of claims 25 to 28, further comprising at least one non-immobilized competing reagent.

30. The kit of claim 29, wherein the at least one non-immobilized competing reagent is lyophilized or in solution.

31. The kit of claim 29 or 30, wherein the non-immobilized competing reagent is a binding partner of salivary amylase.

32. The kit of any of claims 25 to 31, further comprising a detection reagent that specifically binds to CD20, a detection reagent that specifically binds to IL-18, a detection reagent that specifically binds to CD27, a detection reagent that specifically binds to thyroid peroxidase (TPO), or combination thereof.

33. The kit of claim 32, further comprising a detection reagent that specifically binds to CD20, a detection reagent that specifically binds to IL-18, or both.

34. A method of determining radiation exposure in a human, comprising
a. detecting CD5 in a biological sample of a human,
b. determining if a concentration of CD5 in the biological sample is lower than a control concentration of CD5 in a non-irradiated control sample,
c. if the concentration in the biological sample is lower than in the non-irradiated control sample, reporting that the human was exposed to radiation.

35. The method of claim 34, wherein the biological sample is whole blood, serum, plasma, cerebrospinal fluid, urine, saliva, or an extraction or purification therefrom, or dilution thereof.

36. The method of claim 35, wherein the biological sample is serum or plasma.

37. The method of any of claims 34 to 36, wherein the method comprises measuring one or more additional biomarkers selected from salivary amylase, IL-15, IL-18, Flt-3L and CD20.

38. The method of any of claims 34 to 37, wherein step (a) is performed by immunoassay.

39. The kit of any of claims 25 to 33, wherein the surface is a plate.

40. The kit of claim 39, wherein the plate is stable to storage for about 0 hours to about 72 hours at about 37° C.

41. The kit of claim 40, wherein assay performance of the plate after storage for about 72 hours at about 37° C. does not vary by more than 20% as compared to assay performance of the plate immediately upon removal from storage in a vacuum sealed, desiccated container at about 2° C. to about 8° C.
